(19) United States
(12) Patent Application Publication
(10) Pub. No.: US 2006/0147915 A1
(43) Pub. Date: Jul. 6, 2006

(54) Disease risk estimating method fusing sequence polymorphisms in a specific region of chromosome 19

(75) Inventors: Bjorn Andersen Nexo, Abyhoj (DK); Ulla Vogel, Bronshoj (DK); Eszter Rockenbauer, Copenhagen (DK); Zuzanna Katarzyna Bukowy, Aarhus C (DK)

Correspondence Address: BROWDY AND NEIMARK, P.L.L.C.;624 NINTH STREET, NW, SUITE 300, WASHINGTON, DC, 20001-5303, US

(73) Assignee: AARHUS UNIVERSITET, ARHUS (DK)

(21) Appl. No.: 10/519,505

(22) PCT Filed: Jun. 27, 2003
(86) PCT No.: PCT/DK03/00448

§ 371 Date: Dec. 27, 2004

(30) Foreign Application Priority Data
Jun. 27, 2002 (DK) PA 2002 01005
Oct. 7, 2002 (DK) PA 2002 01500
Feb. 25, 2003 (DK) PA 2003 00289
Apr. 29, 2003 (DK) PA 2003 00639

(51) Int. Cl. C12Q 1/68

(52) U.S. Cl. 435/6

(57) ABSTRACT

The present invention provides methods and compositions for identifying human subjects with an increased risk of having or developing disease. In particular, this invention relates to the identification and characterization of polymorphisms in the human chromosome 19q, the region r located approximately 19q13.2-3 correlated with increased risk of developing disease, in particular cancer and the responsiveness of a subject to various treatments for cancer. An allele in the r region can be identified as correlated with an increased risk of developing disease, in particular cancer, the prognosis of developed disease, in particular cancer, and responsiveness to disease treatment, in particular cancer treatment on the basis of statistical analyses of the incidence of a particular allele in individuals diagnosed with disease, in particular cancer. The invention further relates to probes, and kits comprising the probes useful in the diagnostic.

The present invention provides methods and compositions for identifying human subjects with an increased risk of having or developing disease. In particular, this invention relates to the identification and characterization of polymorphisms in the human chromosome 19q, the region r located approximately 19q13.2-3 correlated with increased risk of developing disease, in particular cancer and the responsiveness of a subject to various treatments for cancer.

### BACKGROUND

DNA polymorphisms provide an efficient way to study the association of genes and diseases by analysis of linkage and linkage disequilibrum. With the sequencing of the human genome a myriad of hitherto unknown genetic polymorphisms among people have been detected. Most common among these are the single nucleotide polymorphisms, also called SNPs, of which several millions are known. Other examples are variable number of tandem repeat polymorphisms, insertions, deletions and block modifications. Tandem repeats often have multiple different alleles (variants), whereas the other groups of polymorphisms usually just have two alleles. Some of these genetic polymorphisms probably play a direct role in the biology of the individuals, including their risk of developing disease, but the virtue of the majority is that they can serve as markers for the surrounding DNA, and thus serve as leads during as search for a causative gene polymorphism, as substitutes in the evaluation of its role in health and disease, and as substitutes in the evaluation of the genetic constitution of individuals.

The association of an allele of one sequence polymorphism with particular alleles of other sequence polymorphisms in the surrounding DNA has two origins, known in the genetic field as linkage and linkage disequilibrium, respectively. Linkage arises because large parts of chromosomes are passed unchanged from parents to offspring, so that minor regions of a chromosome tend to flow unchanged from one generation to the next and also to be similar in different branches of the same family. Linkage is gradually eroded by recombination occurring in the cells of the germline, but typically operates over multiple generations and distances of a number of million bases in the DNA.

Linkage disequilibrium deals with whole populations and has its origin in the (distant) forefather in whose DNA a new sequence polymorphism arose. The immediate surroundings in the DNA of the forefather will tend to stay with the new allele for many generations. Recombination and changes in the composition of the population will again erode the association, but the new allele and the alleles of any other polymorphism nearby will often be partly associated among unrelated humans even today. A crude estimate suggests that alleles of sequence polymorphisms with distances less that 10000 bases in the DNA will have tended to stay together since modern man arose. Linkage disequilbrium in limited populations, for instance Europeans, often extends over longer distances. This can be the result of newer mutations, but can also be a consequence of one or more “bottlenecks” with small population sizes and considerable inbreeding in the history of the current population. Two obvious possibilities for “bottlenecks” in Europeans are the exodus from Africa and the repopulation of Europe after the last ice age.

Linkage disequilibrium is the results of many stochastic events and as such subject to statistical variation occasionally resulting in discontinuities, lack of a monotonic relationship between association and distance and differences between people of different ethnicity. Therefore, it is often advantageous to study more that one sequence polymorphism in a given region. This also allows for further definition of the genetic surroundings of the biologically relevant polymorphism by combining the associated alleles of the different markers into a socalled haplotype.

Humans in general carry two copies of each human chromosome in each cell. There are exceptions to this rule, not relevant to this application. We therefore speak about genotypes i.e. the combined analysis of both chromosomes at a given sequence polymorphism. The resulting genotypes of a person, analysed for instance on DNA from peripheral blood leukocytes, are inherently very stable over time. Therefore, this type of analysis can be performed any time in the life of a person and will be applicable to this person for his or her entire life. By the same token such genetic analyses are ideally suited to predict future risks of disease.

A variety of investigations suggest that many diseases in part are determined by the genetic constitution of the individual. One group of genes in particular has been associated with rare genetic predispositions to cancer. These are the genes involved in maintaining the integrity of a persons DNA, the so-called DNA repair genes. One set of such genes are the XP genes which participate in nucleotide excision repair, and, when mutated, give rise to a 1000 fold increased risk of getting skin cancer. For this reason we have previously investigated single nucleotide polymorphisms in one DNA repair gene XPD for association with risk of skin cancer in a cohort of Caucasian Americans, and found that one allele of the sequence polymorphism called XPDe6 was associated with a moderately increased risk of getting basal cell carcinoma, the most common form of skin cancer. Later other groups have studied the association between sequence polymorphisms in this and other DNA repair genes and various forms of cancer. Some have reported positive results.

Very little is known about the function of the gene RAI. It was cloned because its protein product binds to and inhibits ReIA of the transcription regulator NF-kappaB.

### SUMMARY OF THE INVENTION

The present invention relates in a first aspect to a group of nucleic acid sequences found to be associated with disease, in particular cancer. The invention further relates to transcriptional and translational products of said sequence. An allele in the r region can be identified as correlated with an increased risk of developing disease, in particular cancer, the prognosis of developed disease, in particular cancer, and responsiveness to disease treatment, in particular cancer treatment on the basis of statistical analyses of the incidence of a particular allele in individuals diagnosed with disease, in particular cancer.

Thus, in a first aspect the invention relates to a method for estimating the disease risk of an individual comprising
providing a sample from said individual,
assessing in the genetic material including human genes in said sample a sequence polymorphism
   in a region corresponding to SEQ ID NO: 2, or a part thereof, or
   in a region complementary to SEQ ID NO: 2, or apart thereof, or
   in a transcription product from a sequence in a region corresponding to SEQ ID NO: 2, or a part thereof, or
   or translation product from a sequence in a region corresponding to SEQ ID NO: 2, or a part thereof,
   obtaining a sequence polymorphism response,
estimating the disease risk of said individual based on the sequence polymorphism response.

Preferably the invention relates to a method for estimating the disease risk of an individual comprising
providing a sample from said individual,
assessing in the genetic material including human genes in said sample a sequence polymorphism
   in a region corresponding to SEQ ID NO: 1, or a part thereof, or
   in a region complementary to SEQ ID NO: 1, or a part thereof, or
   in a transcription product from a sequence in a region corresponding to SEQ ID NO: 1, or a part thereof, or
   or translation product from a sequence in a region corresponding to SEQ ID NO: 1, or a part thereof,
   obtaining a sequence polymorphism response,
estimating the disease risk of said individual based on the sequence polymorphism response.

The estimation of the disease risk of an individual can involve the comparison of the number and/or kind of polymorphic sequences identified with a predetermined disease risk profile. Such a profile can be based on statistical data obtained for a relevant reference group of individuals. In particular the disease is a proliferative disease, such as cancer.

The sequence of the r region is set forth as SEQ ID NO 1, originating from the cloning of human chromosome 19q published as part of the contig NT_{—}011109 in the database of human sequences established by National Center for Biotechnology Information and located on the internet at http://www.ncbi.nlm.nih.gov/genome/guide/human/

The presence of an allele is determined by determining the nucleic acid sequence of all or part of the region according to standard molecular biology protocols well known in the art as described for example in Sambrook et al. (1989) and as set forth in the Examples provided herein or products of the nucleic acid sequences.

In particular, the nucleic acid molecules of the present invention represent in a first aspect nucleic acid sequences forming part of the region r corresponding to position 1522-37752 of SEQ ID NO: 1, and preferably to certain nucleic acid sequences within the gene referred to herein as RAI. As demonstrated in the Examples presented below, the RAI gene is in particular associated with human cancer diseases.

Furthermore, the invention relates to a method for estimating the disease prognosis of an individual comprising
providing a sample from said individual,
assessing in the genetic material including human genes in said sample a sequence polymorphism
   in a region corresponding to SEQ ID NO: 1 or SEQ ID NO: 2, or a part thereof, or
   in a region complementary to SEQ ID NO: 1 or SEQ ID NO: 2, or a part thereof, or
   in a transcription product from a sequence in a region corresponding to SEQ ID NO: 1 or SEQ ID NO: 2, or a part thereof, or
   or translation product from a sequence in a region corresponding to SEQ ID NO: 1 or SEQ ID NO: 2, or a part thereof,
   obtaining a sequence polymorphism response,
estimating the disease prognosis of said individual based on the sequence polymorphism response.

The estimation of the disease prognosis of an individual can involve the comparison of the number and/or kind of polymorphic sequences identified with a predetermined disease prognosis profile. Such a profile can be based on statistical data obtained for a relevant reference group of individuals.

Additionally provided is a method of identifying a human subject as having an increased likelihood of responding to a treatment, comprising a) correlating the presence of an r region allele genotype with an increased likelihood of responding to treatment; and b) determining the r region allele genotype of the subject, whereby a subject having an r region allele genotype correlated with an increased likelihood of responding to treatment is identified as having an increased likelihood of responding to treatment.

Thus, the present invention also relates to method for estimating a treatment response of an individual suffering from disease to a disease treatment, comprising
providing a sample from said individual,
assessing in the genetic material including human genes in said sample a sequence polymorphism
   in a region corresponding to SEQ ID NO: 1 or SEQ ID NO: 2, or a part thereof, or
   in a region complementary to SEQ ID NO: 1 or SEQ ID NO: 2, or a part thereof, or
   in a transcription product from a sequence in a region corresponding to SEQ ID NO: 1 or SEQ ID NO: 2, or a part thereof, or
   or translation product from a sequence in a region corresponding to SEQ ID NO: 1 or SEQ ID NO: 2, or a part thereof,
   obtaining a sequence polymorphism response,
estimating the individual's response to the disease treatment based on the sequence polymorphism response.

The estimation of the individual's response to disease treatment can involve the comparison of the number and/or kind of polymorphic sequences identified with a predetermined cancer treatment response profile. Such a profile can be based on statistical data obtained for a relevant reference group of individuals. In particular the disease is a proliferative disease, such as cancer.

The invention also comprises primers or probes for use in the invention, as well as kits including these. The primers and/or probes are preferably capable of hybridising to SEQ ID NO:1 or SEQ ID NO: 2, or a part thereor, in particularly the r region, or a part thereof, under stringent conditions, as well as to a sequence complementary thereto.

Furthermore, the invention also relates to cloning vectors and expression vectors containing the nucleic acid molecules of the invention, as well as hosts which have been transformed with such nucleic acid molecules, including cells genetically engineered to contain the nucleic acid molecules of the invention, and/or cells genetically engineered to express the nucleic acid molecules of the invention. The nucleic acids are preferably isolated from the r region and preferably contain one or more sequence polymorphisms as described herein below in more detail. In addition to host cells and cell lines, hosts also include transgenic non-human animals (or progeny thereof).

In particular, the present invention is based on the discovery of the correlation with single nucleotide polymorphisms (SNPs) and/or tandem repeats in the r region and disease. Thus, SNPs have been found in the r region as shown in table 1. However, the present invention is not limited to the SNPs shown in table 1, but does include any SNP in the region. Tandem repeats have been found in the r region as shown in table 2. However, the present invention is not limited to the tandem repeats shown in table 2, but does include any tandem repeat in the region.

The term human includes both a human having or suspected of having a disease and an a-symptomatic human who may be tested for predisposition or susceptibility to disease. At each position the human may be homozygous for an allele or the human may be a heterozygote.

### DRAWINGS

FIG. 1 shows a subregion of chromosome 19q

FIG. 2 shows odds ratios and p-values for individual sequence variations in relation to risk of basal cell carcinoma

FIG. 3 shows odds p-values for association of different sequence variations with risk of basal cell carcinoma among psoriatic Danes

FIG. 4 shows regions S1, S2 and S3 of SEQ ID NO: 2.

### DETAILED DESCRIPTION OF THE INVENTION

The present invention relates to a characterization of a person's present and/or future risk of getting certain forms of disease, in particular a proliferative disease, such as cancer. The characterization is based on the analysis of sequence polymorphisms in a region of chromosome 19q in the person.

A number of polymorphisms in the chromosomal region 19q13.2-3 have been identified and characterised. Surprisingly, the sequence polymorphisms with strongest association to disease appeared to be located outside the gene XPD. More specifically, the sequences were located in a sub-region between the gene XPD and the gene ERCC1, and seemed to have a maximum in or around the gene RAI (See Example 1). For persons getting their skin cancer relatively early (before 50 years of age), it was found that predictions got better (Example 2) and when two sequence polymorphisms in RAI were combined, the prediction of early skin cancer got even better (Example 3). It was also possible to combine sequence polymorphisms in RAI with sequence polymorphisms outside the region and get highly positive results (Example 4).

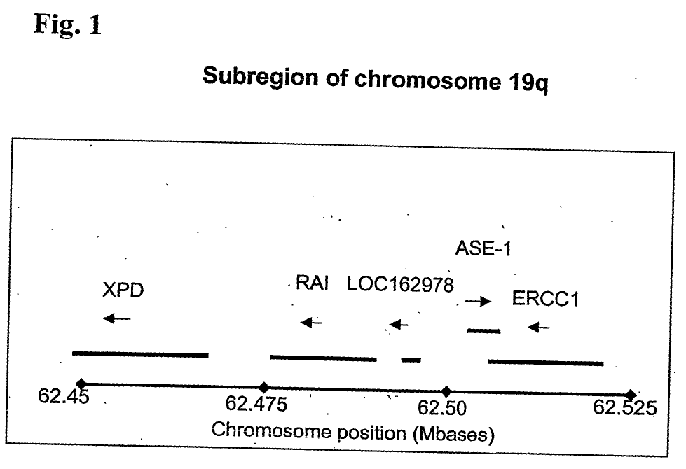

The region of chromosome 19q, more precisely the region located in 19q13.2-3, with which the present invention is concerned, is depicted in FIG. 1 as it is presently known together with the presently known or suspected genes. The arrows indicate the directions of transcription of the genes. The absolute chromosome positions shown are from the particular build of NCBI's map of chromsome 19, and will probably change with time.

The region s stretches from the XPD gene to approximately the end of ERCC1 and includes the region r and is defined by SEQ ID NO: 2. In the present context the region s means SEQ ID NO: 2 and complementary sequence as well as transcriptional products and translational products thereof.

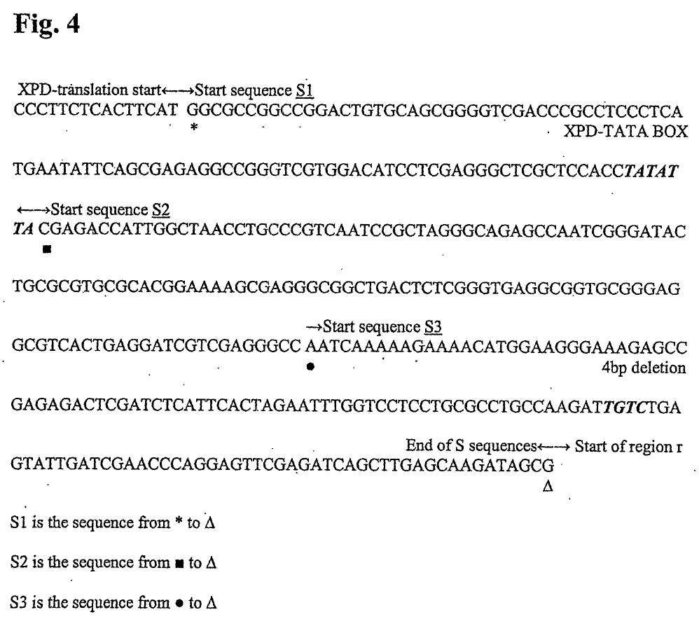

One preferred section of the region s is S1 as shown in FIG. 4, more preferred S2 as shown in FIG. 4, most preferred S3 as shown in FIG. 4.

The region r stretches from the beginning of, but not including the XPD gene, to approximately the end of ERCC1 and includes the genes RAI, LOC162978, and ASE-1. More specifically r is bounded by and includes the following two sequences: AGAACCCCCG CCCCTCCACC TCGTCTCAAA and TCCCTCCCCA GAGACTGCAC CAGCGCAGCC, and is defined by SEQ ID NO: 1.

In the present context the region r means SEQ ID NO: 1 and complementary sequence as well as transcriptional products and translational products thereof.

One preferred section of the region r stretches approximately from the end of RAI to the end of ASE-1 and includes the genes RAI, LOC162978, and ASE-1. More specifically, this section of r is bounded by and includes the following sequences: GAAGTGAGCC AAGATCACGC CACTGCACTC and GTGCCCACCT GGGCCACCAG AAGGTGACAC. In the present context the region r means SEQ ID NO: 1 bases 1522-37752 and complementary sequence as well as transcriptional products and translational products thereof.

Finally, in the claims the gene RAI is defined as including transcribed sequences of the gene plus a 1500 base upstream promoter region. More specifically RAI is bounded by and includes the following sequences: CATAACCACA ATGATGAGCA TGTATTGAGT and ATGTTGTCCA GGCTGGTCTT GAACTCCTGA. In the present context this section of the region relates to SEQ ID NO: 1 bases 7761-22885 and complementary sequence as well as transcriptional products and translational products thereof.

Modifications to the human genome map are known to occur from time to time. It is therefore possible that the defining sequences quoted above will change slightly in future maps.

Fragments or parts of the region s or r as used herein relates to any fragment of at least 100 nucleic acid redues in length, or mutiples of 100 nucleic acid residues in length, starting from SEQ ID NO: 1 position 1, 100, 200, 300, 400, 500, 600, 700, 800, 900, 1000, 1100, 1200, 1300, 1400, 1500, 1600, 1700, 1800, 1900, 2000, 2100, 2200, 2300, 2400, 2500, 2600, 2600, 2700, 2800, 2900, 3000, and so forth, each fragment starting position having an increment of 100 nucleic acid residues. Multiples are preferably multiples of e.g. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 and 50.

For fragments starting at position 1, the length of said fragments will thus be e.g. 100, 200, 300, 400, 500, 600, 700, 800, 900, 1000, 1100, 1200, 1300, 1400, 1500, 1600, 1700, 1800, 1900, 2000, 2100, 2200, 2300, 2400, 2500, 2600, 2600, 2700, 2800, 2900, 3000, and so forth, using suitable multiplicators as listed herein above.

For fragments starting at position 100, the length of said fragments will thus be e.g. 100, 200, 300, 400, 500, 600, 700, 800, 900, 1000, 1100, 1200, 1300, 1400, 1500, 1600, 1700, 1800, 1900, 2000, 2100, 2200, 2300, 2400, 2500, 2600, 2600, 2700, 2800, 2900, 3000, and so forth, using suitable multiplicators as listed herein above.

For fragments starting at position 7700, the length of said fragments will thus be e.g. 100, 200, 300, 400, 500, 600, 700, 800, 900, 1000, 1100, 1200, 1300, 1400, 1500, 1600, 1700, 1800, 1900, 2000, 2100, 2200, 2300, 2400, 2500, 2600, 2600, 2700, 2800, 2900, 3000, 3500, 4000, 4500, 5000, 5500, 6000, 6500, 7000, 7500, 8000, 8500, 9000, 9500, 10000, 10500, 11000, 11500, 12000, 12500, 13000, 13500, 14000, 14500, 15000, and so forth, using suitable multiplicators such as e.g. the ones listed herein above.

The nucleic acid sequences according to the present invention makes it possible to estimate cancer risk in an individual by using sequence polymorphisms originating from a specific region of chromosome 19.

Estimation of disease risks has a number of important applications, which in the following is exemplified with respect to cance, but also apply to other disease, as described herein:

(1) Individuals with reasons to suspect that they are at risk for getting cancer would be able to clarify their situation and, if possible, take protective action. Alternatively, ant-cancer campaigns, companies, hospitals or other institutions could offer a service to help people clarify their situation. It would for instance be possible to test persons, when they got their first basal cell carcinoma, which is often recurrent and also is a moderate predictor for other cancers. If the persons were in a high-risk group, one could then advice them about, or they could of their own accord choose, risk-reducing behaviour, such as avoidance of excessive sun-exposure, abstaining from smoking etc. About 5 percent of the Danish population will at some point in their life get a basal cell carcinoma.

(2) Anti-cancer campaigns, companies, hospitals or other institutions would be able to define relevant target subpopulations and focus information on risk-reducing behaviour on these persons. They might perhaps also be in a position to inform the remainder of the population that they need not worry. Lung cancer affects approximately 10-15 percent of smokers and thus approximately 5 percent of the population, somewhat varying from country to country. Malignant melanoma, a sun-induced, often lethal form of skin cancer, affects approximately 700 persons a year in Denmark or about 1 percent of the Danish population.

(3) The drugs used in cancer treatment are often carcinogenic themselves and individual responses to them vary considerably, both with respect to tolerance to the treatment and with respect to efficacy of the treatment. It is an obvious possibility that the region of chromosome 19 here dealt with, which contains DNA repair genes known to modulate carcinogen responses, also modulates response to anti-cancer agents. Hence, analysis of the region may facilitate better choices of treatment for cancer, and/or help predict the future course of disease.

By sequence polymorphism is understood any single nucleotide, tandem repeat, insert, deletion or block polymorphism, which varies among humans, whether it is of known biological importance or not.

Position of Sequence Polymorphism in the Region s and r

In one embodiment of the methods of the invention, preferably the method for diagnosis as described herein, one or more single nucleotide polymorphism(s) at a predetermined position in the region r (SEQ ID NO:1) are identified and used for e.g. cancer risk profiling and/or cancer treatment response profiling. Presently preferred single nucleotide polymorphism(s) are listed in Tables 1a, 1b and 1c, more preferably at least two single nucleotide polymorphism(s) are selected, most preferably at least three single nucleotide polymorphism(s) are selected. However, the present invention relates to any SNP in the r region.

TABLE 1a

| Identification in dbSNP¹ | Position in SEQ ID NO: 1 |
|---|---|
| rs#209725 C/A | ambiguous location |
| rs#2017154 A/C | 12115 |
| rs#2070830 T/G | 14575 |
| rs#959457 C/T | 32446 |
| rs#2336218 C/A | 32447 |
| rs#766934 A/G | 32481 |
| rs#928911 C/T | 32785 |
| rs#1005165 C/T | 33974 |
| rs#1005166 C/T | 34119 |
| rs#1046282 T/C | 35596 |
| rs#2013521 A/T | 36254 |
| rs#2336919 | ambiguous location |
| rs#743571 C/G | 37786 |

TABLE 1b

| | |
|---|---|
| Identification | Position in |
| in dbSNP¹ | SEQ ID NO: 1 |

| | | |
|---|---|---|
| rs#3047560 | ataaaaaaat aaaaaaaa (-/AA) atagccgagc atggtggtgg | 4795-6 |
| rs#5000150 | tgttgtccaa gctggCAGAG (A/G) tttttgtttg tttgtttgag | 6908 |
| rs#4589665 | CCAGGGCATA CAACCAGCAC (T/A) TGATTTTctg tgtgacctca | 20613 |
| rs#4803814 | cctgcttgct tgctttctct (C/T) tctctctttc tttctttctt | 25650 |
| rs#4803815 | cttgcttgct ttctctctct (C/T) tctttctttc tttctttctt | 25654 |
| rs#4572514 | CTGTTCAGGC TGGCGGCTCA (C/T) TTGGATGAAC AGGGAGTGTG | 28691 |
| rs#4802252 | agccaccaca cctggccAAA (C/T) CAGCTATTCT GAAAGGCCCC | 29686 |
| rs#4803816 | GAGCCTATTG TTGGAAAGTT (C/T) TGAGTCCAAG ATTCTATCTT | 29815 |
| rs#4802253 | CCTAACCCAG GGTTGCACTG (C/T) TCTGGAAGTC TAGATGGATG | 29922 |
| rs#4353560 | GTAAGTGACt cttttttttt (C/T) ttttggtaga gatttagtct | 30439 |
| rs#3212989 | TCGGGGACAG GACTG (C/T) GTCTTCTAGA GGCTCAGTGT | 36994 |
| rs#3212999 | TGGCTGAGAC TCAAC (C/T) GTCACCCCCT CCTCTGGCTC | 37068 |
| rs#3212987 | GTGTGACCTC TCTCT (-/TTC) TTCTTCTTCT TCTTCTTGGT | 37431-37433 |
| rs#3212986 | GCTGCTGCTG CTGCT (T/G) CTTCCGCTTC TTGTCCCGGC | 37660 |

¹dbSNP is the database over SNPs established by the National Center for Biotechnology Information and located on the internet at http://www.ncbi.nlm.nih.gov/SNP/.

TABLE 1c

| | | | |
|---|---|---|---|
| Trivial | | | |
| name | rs number | Sequence | Position |

| | | | |
|---|---|---|---|
| XRCC1e10 | 25487 | GGCGGCTGCC CTCCC (A/G) GAGGTAAGGC CTCACACGCC | — |
| CKMe8 | 4884 | AGTTGGAGAA AGGCCAGTCC AT (C/T) GACGACATGA | — |
| XPDe23 | See ref 1 | CGCTG (A/C) AGAGG | |
| XPDe10 | See ref 1 | TGCC (G/A) ACGAA | |
| XPDe6 | See ref 1 | TGCCG (C/A) TTCTA | |
| | 3810366 | CAATCCGCTA GGGCA (C/G) AGCCAATCGG GATACTGCGC | 143 in SEQ NO 2 |
| XPD_4bp | 3916791 | ttcgatcaat actca (-/GACA) atcttggcAG GCGCAGGAGG | 323-326 |
| | | | in SEQ NO 2 |
| XPDi4 | 1618536 | tggctctgaa acttactagc cc (A/G) tatttatgg agagg | — |
| | 3916790 | caggcttgag ccacc (A/G) cgcccggccT GCAAAGCCAT | 137 in SEQ NO 1 |
| | 3916789 | gtagagacag gggtt (T/-) ctccatgttg gtcaggctgg | 232 in SEQ NO 1 |
| | 3916788 | ttagtagaga caggg (T/G) tttctccatg ttggtcaggc | 235 in SEQ NO 1 |
| | 3916787 | gctgcagtga gctgt (-/ACACCTGTGGTCCCAGCTACTCTGG | 632-633 |
| | | AAGCTGAGGTGGGAGGATCGCTTGAGCCCAAGAGGTGGAGGCTGC | in SEQ NO 1 |
| | | AGTGAGCTGT) gactgtgcca ctgcactcca | |
| XPD-5′2 | 2097215 | TGACAGTAGA CATCCTGTCA T (A/G) ATAAGTCttt ttttttt | 1610 in SEQ NO 1 |
| RAI-3′ | 2377328 | GGTTGAGAgg ccaggcg (C/T) ggtgctcacg cctgtaattt | 7199 in SEQ NO 1 |
| RAIe6 | 6966 | ATTAAGTGCC TTCACACAGC (A/T) CTGGTTTAAT GTTTATA | 7887 in SEQ NO 1 |
| RAIi5 | 4410192 | CAGACCTCCC TCTCCCAATA (A/T) AACGGTTTGT CCTGTTGCC | 10609 in SEQ NO 1 |
| RAIi3 | 2017104 | gggaggctcg aggcgggc (A/G) gattgcatga gctcaggatt | 12190 in SEQ NO 1 |
| RAii1 | 1970764 | tgcagtgagc tgagatcgc (A/G) ccactgcact ccagcctggg | 15798 in SEQ NO 1 |
| RAI-5UTR | 4589665 | CAGGGCATA CAACCAGCAC (A/T) TGATTTTctg tgtgacctca | — |
| RAI-5′2 | 4803814 | cctgcttgct tgctttctct (C/T) tctctctttc tttctttc | 25650 in SEQ NO 1 |
| RAI-5′3 | 4803815 | cttgcttgct ttctctctct (C/T) tctttctttc tttctttc | 25564 in SEQ NO 1 |
| RAI-5′ | 4572514 | CTGTTCAGGC TGGCGGCTCA (C/T) TTGGATGAAC AGGGAGTG | 28691 in SEQ NO 1 |
| ASE1-5′2 | 2226949 | TCTTAGGACG CATGGGGGT (G/T) GAGAGAACGG GGAGATAGA | 32035 in SEQ NO 1 |
| | 4803817 | TCGGGGATTC GAACCCCTAT (r) CTACCCAAAG ACTCGGCTTC | 32885 in SEQ NO 1 |
| ASE1e1 | 967591 | GCAGCCCGGG CTACAGGGTT (A/G) CCTGAGGTGT GGGTCCCAGG | 34858 in SEQ NO 1 |
| | 5828233 | aagactctct caaaaaaaaa (A/-) caaaaaaaaa acaaaaaaC | 36241 in SEQ NO 1 |
| | | CTTCCCTCTC CTGTTCCACT | |
| ASE1e3a | 735482 | AAGCCCAAAG GGA (A/C) AGAAACCTTC GAGCCAGAAG | 36926 in SEQ NO 1 |
| ERCC1-3′ | 762562 | AGCCAGAAGG AGCG (A/G) AGCCTCAGGC CCAGGCAGCT | 37267 in SEQ NO 1 |
| ASE1e3b | 2336219 | AGAAAGAAAA ACAGCAA (A/G) ATGCCACAGT GGAGCCAGAG | — |
| ERCC1e4 | See ref 1 | GGCAC (G/A) TTGCG | |
| ERCC1e3 | See ref 1 | GGGCA (C/T) GTGGC | |
| FOSBe4 | 1049698 | CACCCTTTTT TTGGGGTGCC (C/T) AGGTTGGTTT CCCCTGCA | — |
| SLC1A5e8 | 1060043 | GCAGGACTCC TCCAAAATTA (C/T) GTGGACCGTA CGGAGTCG | — |
| LIG1e6 | 20580 | AGAGGCTGAA GTGGC (A/C) ACAGAGAAGG AAGGAGAAGA | — |
| GLTSCR1e1 | 1035938 | ccTGAGCAAA CCCATGAG (C/T) GTCCACCTCC TGAACCAAGG | — |

More preferably single nucleotide polymorphism(s) are listed below, more preferably at least two single nucleotide polymorphism(s) are selected, most preferably at least three single nucleotide polymorphism(s) are selected:

| | |
|---|---|
| rs#2017154 A/C | 12115 |
| rs#2070830 T/G | 14575 |
| rs#959457 C/T | 32446 |
| rs#2336218 C/A | 32447 |
| rs#766934 A/G | 32481 |
| rs#928911 C/T | 32785 |
| rs#1005165 C/T | 33974 |
| rs#1005166 C/T | 34119 |

| | | |
|---|---|---|
| rs#4589665 | CCAGGGCATA CAACCAGCAC (T/A) TGATTTTctg tgtgacctca | 20613 |
| rs#4803814 | cctgcttgct tgctttctct (C/T) tctctctttc tttctttctt | 25650 |
| rs#4803815 | cttgcttgct ttctctctct (C/T) tctttctttc tttctttctt | 25654 |
| rs#4572514 | CTGTTCAGGC TGGCGGCTCA (C/T) TTGGATGAAC AGGGAGTGTG | 28691 |
| rs#4802252 | agccaccaca cctggccAAA (C/T) CAGCTATTCT GAAAGGCCCC | 29686 |
| rs#4803816 | GAGCCTATTG TTGGAAAGTT (C/T) TGAGTCCAAG ATTCTATCTT | 29815 |
| rs#4802253 | CCTAACCCAG GGTTGCACTG (C/T) TCTGGAAGTC TAGATGGATG | 29922 |
| rs#4353560 | GTAAGTGACt cttttttttt (C/T) ttttggtaga gatttagtct | 30439 |
| rs#3212989 | TCGGGGACAG GACTG (C/T) GTCTTCTAGA GGCTCAGTGT | 36994 |

| | | | |
|---|---|---|---|
| RAI-3′ | 2377328 | GGTTGAGAgg ccaggcg (C/T) ggtgctcacg cctgtaattt | 7199 in SEQ NO 1 |
| RAIe6 | 6966 | ATTAAGTGCC TTCACACAGC (A/T) CTGGTTTAAT GTTTATAA | 7887 in SEQ NO 1 |
| RAIi5 | 4410192 | CAGACCTCCC TCTCCCAATA (A/T) AACGGTTTGT TCCTGTTGCC | 10609 in SEQ NO 1 |
| RAIi3 | 2017104 | gggaggctcg aggcgggc (A/G) gattgcatga gctcaggatt | 12190 in SEQ NO 1 |
| RAii1 | 1970764 | tgcagtgagc tgagatcgc (A/G) ccactgcact ccagcctggg | 15798 in SEQ NO 1 |
| RAI-5UTR | 4589665 | CAGGGCATA CAACCAGCAC (A/T) TGATTTTctg tgtgacctca | — |
| RAI-5′2 | 4803814 | cctgcttgct tgctttctct (C/T) tctctctttc tttctttc | 25650 in SEQ NO 1 |
| RAI-5′3 | 4803815 | cttgcttgct ttctctctct (C/T) tctttctttc tttctttc | 25564 in SEQ NO 1 |
| RAI-5′ | 4572514 | CTGTTCAGGC TGGCGGCTCA (C/T) TTGGATGAAC AGGGAGTG | 28691 in SEQ NO 1 |
| ASE1-5′2 | 2226949 | TCTTAGGACG CATGGGGGT (G/T) GAGAGAACGG GGAGATAGA | 32035 in SEQ NO 1 |
| | 4803817 | TCGGGGATTC GAACCCCTAT (r) CTACCCAAAG ACTCGGCTTC | 32885 in SEQ NO 1 |
| ASE1e1 | 967591 | GCAGCCCGGG CTACAGGGTT (A/G) CCTGAGGTGT GGGTCCCAGG | 34858 in SEQ NO 1 |
| | 5828233 | aagactctct caaaaaaaaa (A/-) caaaaaaaaa atcaaaaaaC | 36241 in SEQ NO 1 |
| | | CTTCCCTCTC CTGTTCCACT | |
| ASE1e3a | 735482 | AAGCCCAAAG GGA (A/C) AGAAACCTTC GAGCCAGAAG | 36926 in SEQ NO 1 |

Most preferably single nucleotide polymorphism(s) are those listed below, more preferably at least two single nucleotide polymorphism(s) are selected, most preferably at least three single nucleotide polymorphism(s) are selected:

| | | | |
|---|---|---|---|
| RAI-3′ | 2377328 | GGTTGAGAgg ccaggcg (C/T) ggtgctcacg cctgtaattt | 7199 in SEQ NO 1 |
| RAIe6 | 6966 | ATTAAGTGCC TTCACACAGC (A/T) CTGGTTTAAT GTTTATAA | 7887 in SEQ NO 1 |
| RAIi5 | 4410192 | CAGACCTCCC TCTCCCAATA (A/T) AACGGTTTGT TCCTGTTGCC | 10609 in SEQ NO 1 |
| RAIi3 | 2017104 | gggaggctcg aggcgggc (A/G) gattgcatga gctcaggatt | 12190 in SEQ NO 1 |
| RAii1 | 1970764 | tgcagtgagc tgagatcga (A/G) ccactgcact ccagcctggg | 15798 in SEQ NO 1 |
| RAI-5UTR | 4589665 | CAGGGCATA CAACCAGCAC (A/T) TGATTTTctg tgtgacctca | — |
| RAI-5′2 | 4803814 | cctgcttgct tgctttctct (C/T) tctctctttc tttctttc | 25650 in SEQ NO 1 |
| RAI-5′3 | 4803815 | cttgcttgct ttctctctct (C/T) tctttctttc tttctttc | 25564 in SEQ NO 1 |
| RAI-5′ | 4572514 | CTGTTCAGGC TGGCGGCTCA (C/T) TTGGATGAAC AGGGAGTG | 28691 in SEQ NO 1 |

In a preferred embodiment at least one of the following combination of single nucleotide polymorphisms is included in the methods:

| Number | 1st SNP | 2nd SNP | 3rd SNP |
|---|---|---|---|
| 1 | XPDe23 | XPDe6 | RAI-5′2 |
| 2 | XPDe23 | XPD_4bp | RAI-5′2 |
| 3 | XPDe23 | RAIi3 | ASE1e3a |
| 4 | XPDe23 | RAIi1 | RAI-5′2 |
| 5 | XPDe10 | XPD_4bp | RAI-5′2 |
| 6 | XPDe10 | RAIi1 | RAI-5′2 |
| 7 | XPDe6 | Xpd_$$bp | RAI-5′2 |
| 8 | XPDe6 | XPD_4bp | ERCC1e4 |
| 9 | XPDe6 | RAIi1 | RAI-5′2 |
| 10 | XPDe6 | RAIi1 | ASE1e3b |
| 11 | XPDe6 | RAI-5′2 | ASE1e3b |
| 12 | XPDe6 | RAI-5′2 | ERCC1e4 |
| 13 | XPD_4bp | XPD-5′2 | ASE1e3b |
| 14 | XPD_4bp | XPD-5′2 | ERCC1e4 |
| 15 | XPD_4bp | RAIi3 | RAI-5′2 |
| 16 | XPD_4bp | RAIi3 | ASE1e3b |
| 17 | XPD_4bp | RAIi3 | ERCC1e4 |
| 18 | XPD_4bp | RAIi1 | RAI-5′2 |
| 19 | XPD_4bp | RAIi1 | ASE1e3b |
| 20 | XPD_4bp | RAIi1 | ERCC1e4 |
| 21 | XPD_4bp | RAI-5′2 | ERCC1e4 |
| 22 | XPD_4bp | ASE1e3a | ASE1e3b |
| 23 | XPD_4bp | RAI-5′2 | |
| 24 | XPD_4bp | ASE1e3b | |
| 25 | XPD_4bp | ERCC1e4 | |
| 26 | XPD-5′2 | RAIi3 | RAIi1 |
| 27 | XPD-5′2 | RAIi1 | RAI-5′2 |
| 28 | XPD-5′2 | RAIi1 | ERCC1e4 |
| 29 | XPD-5′2 | ASE1e3a | ASE1e3b |
| 30 | RAIe6 | RAIi1 | ASE1e1 |
| 31 | RAIe6 | RAIi1 | ASE1e3a |
| 32 | RAIe6 | RAIi1 | RAI-5′ |
| 33 | RAIe6 | RAIi1 | ERCC1-3′ |
| 34 | RAIe6 | RAIi1 | ASE1e3b |
| 35 | RAIe6 | RAI-5′ | ASE1e3b |
| 36 | RAIe6 | ASE1e1 | ERCC1-3′ |
| 37 | RAIe6 | ASE1e3a | ERCC1-3′ |
| 38 | RAIe6 | ERCC1-3′ | ERCC1e4 |
| 39 | RAIe6 | RAIi1 | |
| 40 | RAIi3 | RAI-5′ | ERCC1-3′ |
| 41 | RAIi3 | ASE1e1 | ERCC1-3′ |
| 42 | RAIi3 | | |
| 43 | RAIi1 | RAI-5′2 | ASE1e3b |
| 44 | RAIi1 | RAI-5′2 | ERCC1e4 |
| 45 | RAIi1 | RAI-5′2 | RAI-5′ |
| 46 | RAIi1 | ASE1-5′2 | ASE1e1 |
| 47 | RAIi1 | ASE1-5′2 | ASE1e3a |
| 48 | RAIi1 | ASE1-5′2 | ERCC1-3′ |
| 49 | RAIi1 | RAI-5′ | ASE1e3a |
| 50 | RAIi1 | RAI-5′ | ERCC1-3′ |
| 51 | RAIi1 | RAI-5′ | ASE1e3b |
| 52 | RAIi1 | RAI-5′ | ERCC1e4 |
| 53 | RAIi1 | ASE1e1 | ASE1e3a |
| 54 | RAIi1 | ASE1e1 | ERCC1-3′ |
| 55 | RAIi1 | ASE1e1 | ASE1e3b |
| 56 | RAIi1 | ASE1e1 | ERCC1e4 |
| 57 | RAIi1 | ASE1e3a | ERCC1-3′ |
| 58 | RAIi1 | ASE1e3a | ASE1e3b |
| 59 | RAIi1 | ASE1e3a | ERCC1e4 |
| 60 | RAIi1 | ERCC1-3′ | ASE1e3b |
| 61 | RAIi1 | RAI-5′2 | |
| 62 | RAIi1 | ASE1-5′2 | |
| 63 | RAIi1 | ASE1e1 | |
| 64 | RAIi1 | ASE1e3a | |
| 65 | RAIi1 | ERCC1-3′ | |
| 66 | RAIi1 | ASE1e3b | |
| 67 | RAIi1 | | |
| 68 | RAI-5′2 | ASE1e3a | ASE1e3b |
| 69 | RAI-5′ | ASE1e3a | |
| 70 | RAI-5′ | ASE1e3b | |
| 71 | RAI-5′ | | |
| 72 | ASE1-5′2 | RAI-5′ | ASE1e3a |
| 73 | ASE1-5′2 | ASE1e1 | ASE1e3a |
| 74 | ASE1e1 | ASE1e3a | ASE1e3b |
| 75 | ASE1e1 | ASE1e3a | |
| 76 | ASE1e1 | ASE1e3b | |
| 77 | ASE1e3a | ERCC1-3′ | ASE1e3b |
| 78 | ERCC1-3′ | ASE1e3b | ERCC1e4 |
| 79 | ERCC1-3′ | ERCC1e4 | |

In another embodiment of the invention preferably the method described herein is one in which the tandem repeat is at a position as described in Table 2:

TABLE 2
Identification in uniSTS²

D19S908
STS-W67936
D19S543
D19S393
STS-R48186
GDB: 181915
RH47033
GDB: 190019
²UniSTS is a database of unique sequence tag sites established by National Center for Biotechnology Information and located on the internet at http://www.ncbi.nlm.nih.gov/entrez/query.fcgi?db=unists

In another embodiment of the invention, the method for diagnosis described herein is preferably one in which the sequence polymorphism is in region r. Testing for the presence of the RAI gene allele is especially preferred because, without wishing to be bound by theoretical considerations, of its association with increased risk of cancer (as explained herein).

In one embodiment of the methods of the invention, preferably the method for diagnosis as described herein, one or more single nucleotide polymorphism(s) at a predetermined position in the region s (SEQ ID NO:2) are identified and used for e.g. cancer risk profiling and/or cancer treatment response profiling. Presently preferred polymorphism(s) are the four base pair deletion shown in FIG. 4 corresponding to TGTC. However, the present invention relates to any polymorphism and SNP in the s region.

The sequence polymorphism of the invention comprises at least one base difference, such as at least two base differences, such as at least three base differences, such as at least four base differences. As described above the sequence polymorphism comprises at least one single nucleotide polymorphism, such as at least two single nucleotide polymorphisms, such as at least three single nucleotide polymorphism, such as at least four single nucleotide polymorphism. Also, the sequence polymorphism comprises at least one tandem repeat polymorphism, such as at least two tandem repeat polymorphisms.

Also, the sequence polymorphism may be a combination of single nucleotide polymorphism and tandem repeats, such as one single nucleotide polymorphism and one tandem repeat.

The status of the individual may be determined by reference to allelic variation at one, two, three, four or more of the above loci.

Cell Sample

The cell sample used in the present invention may be any suitable cell sample capable of providing the genetic material for use in the method. In a preferred embodiment, the cell sample is a blood sample, a tissue sample, a sample of secretion, semen, ovum, a washing of a body surface (e.g. a buccal swap), a clipping of a body surface (hairs, or nails), such as wherein the cell is selected from white blood cells and tumour tissue.

It will be appreciated that the test sample may equally be a nucleic acid sequence corresponding to the sequence in the test sample, that is to say that all or a part of the region in the sample nucleic acid may firstly be amplified using any convenient technique e.g. PCR, before use in the analysis of variation in the region.

Detection Methods

Detection may be conducted on the sequence of, SEQ ID NO: 1, SEQ ID NO: 2 or a complementary sequence as well as on translational (mRNA) and transcriptional products (polypeptides, proteins) therefrom.

It will be apparent to the person skilled in the art that there are a large number of analytical procedures which may be used to detect the presence or absence of variant nucleotides at one or more of positions mentioned herein in the r region. Mutations or polymorphisms within or flanking the r region can be detected by utilizing a number of techniques. Nucleic acid from any nucleated cell can be used as the starting point for such assay techniques, and may be isolated according to standard nucleic acid preparation procedures that are well known to those of skill in the art. In general, the detection of allelic variation requires a mutation discrimination technique, optionally an amplification reaction and a signal generation system. Table 3 lists a number of mutation detection techniques, some based on the PCR. These may be used in combination with a number of signal generation systems, a selection of which is listed in Table 4. Further amplification techniques are listed in Table 5. Many current methods for the detection of allelic variation are reviewed by Nollau et al., Clin. Chem. 43, 1114-1120, 1997; and in standard textbooks, for example “Laboratory Protocols for Mutation Detection”, Ed. by U. Landegren, Oxford University Press, 1996 and “PCR”, 2nd Edition by Newton & Graham, BIOS Scientific Publishers Limited, 1997.

TABLE 3
Abbreviations:

| | |
|---|---|
| ALEX | Amplification refractory mutation |
| | system linear extension |
| APEX | Arrayed primer extension |
| ARMS | Amplification refractory mutation system |
| b-DNA | Branched DNA |
| CMC | Chemical mismatch cleavage |
| bp | base pair |
| COPS | Competitive oligonucleotide priming system |
| DGGE | Denaturing gradient gel electrophoresis |
| FRET | Fluorescence resonance energy transfer |
| LCR | Ligase chain reaction |
| MASDA | Multiple allele specific diagnostic assay |
| NASBA | Nucleic acid sequence based amplification |
| OLA | Oligonucleotide ligation assay |
| PCR | Polymerase chain reaction |
| PTT | Protein truncation test |
| RFLP | Restriction fragment length polymorphism |
| SDA | Strand displacement amplification |
| SNP | Single nucleotide polymorphism |
| SSCP | Single-strand conformation polymorphism |
| | analysis |
| SSR | Self sustained replication |
| TGGE | Temperature gradient gel electrophoresis |

Table 4 illustrates various mutation detection techniques capable of being used for SNP detection.

TABLE 4

General techniques: DNA sequencing, Sequencing by hybridisation.
SNAPshot.
Scanning techniques: PJT*, SSCP, DOGE, TGGE, Cleavase,
Heteroduplex analysis, CMC, Enzymatic mismatch cleavage
Hybridisation Based techniques
Solid phase bybridisation: Dot blots, MASDA, Reverse dot blots,
Oligonucleotide arrays (DNA Chips)
Solution phase hybridisation: Taqman - U.S. Pat. No. 5,210,015 &
5,487,972 (Hoff-mann-La Roche), Molecular Beacons - Tyagi
et al (1996), Nature Biotechnology, 14, 303; WO 95/13399
(Public Health Inst., New York), Lightcycler, optionally in
combination with FRET.
Extension Based: ARMS, ALEX - European Patent No. EP 332435 B1
(Zeneca Limited), COPS - Gibbs et al (1989), Nucleic Acids Research,
17, 2347.
Incorporation Based: Mini-sequencing, APEX
Restriction Enzyme Based: RFLP, Restriction site generating PCR
Ligation Based: OLA
Other: Invader assay
Various Signal Generation or Detection Systems is listed below:
Fluorescence: FRET, Fluorescence quenching, Fluorescence polarisation -
United Kingdom Patent No. 2228998 (Zeneca Limited)
Other: Chemiluminescence, Electrochemiluminescence, Raman,
Radioactivity, Colorimetric, Hybridisation protection
assay, Mass spectrometry

Table 5 illustrates examples of further amplification techniques.

TABLE 5
SSR, NASBA, LCR, SDA, b-DNA

Preferred mutation detection techniques include ARMS, ALEX, COPS, Taqman, Molecular Beacons, RFLP, and restriction site based PCR and FRET techniques.

Particularly preferred methods include FRET; taqman, ARMS and RFLP based methods.

In a preferred embodiment, mutations or polymorphisms can be detected by using a microassay of nucleic acid sequences immobilized to a substrate or “gene chip” (see, e.g. Cronin, et al., 1996, Human Mutation 7:244-255).

Further, improved methods for analyzing DNA polymorphisms, which can be utilized for the identification of region r specific mutations, have been described that capitalize on the presence of variable numbers of short, tandemly repeated DNA sequences between the restriction enzyme sites. For example, Weber (U.S. Pat. No. 5,075,217) describes a DNA marker based on length polymorphisms in blocks of (dC-dA)n-(dG-dT)n short tandem repeats. The average separation of (dC-dA)n-(dG-dT)n blocks is estimated to be 30,000-60,000 bp. Markers that are so closely spaced exhibit a high frequency co-inheritance, and are extremely useful in the identification of genetic mutations, such as, for example, mutations within the RAI gene, and the diagnosis of diseases and disorders related to RAI mutations.

Also, Caskey et al. (U.S. Pat. No. 5,364,759) describe a DNA profiling assay for detecting short tri and tetra nucleotide repeat sequences. The process includes extracting the DNA of interest, such as the RAI gene, amplifying the extracted DNA, and labelling the repeat sequences to form a genotypic map of the individual's DNA.

The level of RAI gene expression can also be assayed. For example, RNA from a cell type or tissue known, or suspected, to express the RAI gene may be isolated and tested utilizing hybridization or PCR techniques such as are described, above. The isolated cells can be derived from cell culture or from a patient. The analysis of cells taken from culture may be a necessary step in the assessment of cells to be used as part of a cell-based gene therapy technique or, alternatively, to test the effect of compounds on the expression of the RAI gene. Such analyses may reveal both quantitative and qualitative aspects of the expression pattern of the RAI gene, including activation or inactivation of RAI gene expression.

In one embodiment of such a detection scheme, a cDNA molecule is synthesized from an RNA molecule of interest (e.g., by reverse transcription of the RNA molecule into cDNA). A sequence within the cDNA is then used as the template for a nucleic acid amplification reaction, such as a PCR amplification reaction, or the like. The nucleic acid reagents used as synthesis initiation reagents (e.g., primers) in the reverse transcription and nucleic acid amplification steps of this method are chosen from among the RAI gene nucleic acid reagents described above. The preferred lengths of such nucleic acid reagents are at least 9-30 nucleotides. For detection of the amplified product, the nucleic acid amplification may be performed using radioactively or non-radioactively labeled nucleotides. Alternatively, enough amplified product may be made such that the product may be visualized by standard ethidium bromide staining or by utilizing any other suitable nucleic acid staining method.

Additionally, it is possible to perform such RAI gene expression assays “in situ”, i.e., directly upon tissue sections (fixed and/or frozen) of patient tissue obtained from biopsies or resections, such that no nucleic acid purification is necessary. Nucleic acid reagents such as those described above may be used as probes and/or primers for such in situ procedures (see, for example, Nuovo, G. J., 1992, “PCR In Situ Hybridization: Protocols And Applications”, Raven Press, NY).

Alternatively, if a sufficient quantity of the appropriate cells can be obtained, standard Northern analysis can be performed to determine the level of mRNA expression of the RAI gene.

Activity of the Gene

Another method for detecting sequence polymorphism is by analysing the activity of gene products resulting from the sequences. Accordingly, in one embodiment the detection uses the activity of the RAI gene product as compared to a reference in the method. In particular if the activity of the genes are decreased or increased by at least or about 50%, such as at least or about 40%, for example at least or about 30%, such as at least or about 20%, for example at least or about 10%, such as at least or about 10%, for example at least or about 5%, such as at least or about 2%, it indicates a sequence polymorphism in the gene.

Mutations Outside the Region

The present invention may combine the result of sequence polymorphism within the region r or s with sequence polymorphism outside the region in order to increase the probability of the correlation.

Primers

The primer nucleotide sequences of the invention further include: (a) any nucleotide sequence that hybridizes to a nucleic acid molecule of the region s or r or its complementary sequence or RNA products under stringent conditions, e.g., hybridization to filter-bound DNA in 6× sodium chloride/sodium citrate (SSC) at about 45° C. followed by one or more washes in 0.2×SSC/0.1% SDS at about 50-65° C., or (b) under highly stringent conditions, e.g., hybridization to filter-bound nucleic acid in 6×SSC at about 45° C. followed by one or more washes in 0.1×SSC/0.2% SDS at about 68° C., or under other hybridization conditions which are apparent to those of skill in the art (see, for example, Ausubel F. M. et al., eds., 1989, Current Protocols in Molecular Biology, Vol. I, Green Publishing Associates, Inc., and John Wiley & sons, Inc., New York, at pp. 6.3.1-6.3.6 and 2.10.3). Preferably the nucleic acid molecule that hybridizes to the nucleotide sequence of (a) and (b), above, is one that comprises the complement of a nucleic acid molecule of the region s or r or a complementary sequence or RNA product thereof. In a preferred embodiment, nucleic acid molecules comprising the nucleotide sequences of (a) and (b), comprises nucleic acid molecule of RAI or a complementary sequence or RNA product thereof.

Among the nucleic acid molecules of the invention are deoxyoligonucleotides (“oligos”) which hybridize under highly stringent or stringent conditions to the nucleic acid molecules described above. In general, for probes between 14 and 70 nucleotides in length the melting temperature (TM) is calculated using the formula:
Tm(° C.)=81.5+16.6(log[monovalent cations (molar)])+0.41(% G+C)−(500/N)
where N is the length of the probe. If the hybridization is carried out in a solution containing formamide, the melting temperature is calculated using the equation Tm(° C.)=81.5+16.6(log[monovalent cations (molar)])+0.41(% G+C)−(0.61% formamide)−(500/N) where N is the length of the probe. In general, hybridization is carried out at about 20-25 degrees below Tm (for DNA-DNA hybrids) or 10-15 degrees below Tm (for RNA-DNA hybrids).

Exemplary highly stringent conditions may refer, e.g., to washing in 6×SSC/0.05% sodium pyrophosphate at 37° C. (for about 14-base oligos), 48° C. (for about 17-base oligos), 55° C. (for about 20-base oligos), and 60° C. (for about 23-base oligos).

Accordingly, the invention further provides nucleotide primers or probes which detect the s or r region polymorphisms of the invention. The assessment may be conducted by means of at least one nucleic acid primer or probe, such as a primer or probe of DNA, RNA or a nucleic acid analogue such as peptide nucleic acid (PNA) or locked nucleic acid (LNA). The nucleotide primer or probe is preferably capable of hybridising to a subsequence of the region corresponding to SEQ ID NO: 2 or SEQ ID NO: 1, or a part thereof, or a region complementary to SEQ ID NO: 2 or SEQ ID NO: 1.

According to one aspect of the present invention there is provided an allele-specific oligonucleotide probe capable of detecting a r region polymorphism at one or more of positions in the r region as defined by the positions in SEQ ID NO: 1.

The allele-specific oligonucleotide probe is preferably 5-50 nucleotides, more preferably about 5-35 nucleotides, more preferably about 5-30 nucleotides, more preferably at least 9 nucleotides.

The design of such probes will be apparent, to the molecular biologist of ordinary skill. Such probes are of any convenient length such as up to 50 bases, up to 40 bases, more conveniently up to 30 bases in length, such as for example 8-25 or 8-15 bases in length. In general such probes will comprise base sequences entirely complementary to the corresponding wild type or variant locus in the region. However, if required one or more mismatches may be introduced, provided that the discriminatory power of the oligonucleotide probe is not unduly affected. The probes of, the invention may carry one or more labels to facilitate detection.

In one embodiment, the primers and/or probes are capable of hybridizing to and/or amplifying a subsequence hybridizing to a single nucleotide polymorphism containing the sequence shown herein selected from the group of subsequences below or a sequence complementary thereto, wherein the polymorphism is denoted as for example T/C:

| | |
|---|---|
| 1. | GCTCTGAAAC TTACTAGCCC (A/G) GTATTTATGG |
| | AGAGGCATTT |
| 2. | GTGGTCAAAT TCTCATTCAT CGTGG (T/C) CCAGGCAAGC |
| | ACACTTCCTC |
| 3. | ACCCTGAGGT GAGCACCTGT TCCTT (C/T) TCCTTGCCCT |
| | TAGCCCAGAG GTAGA |
| 4. | GGGCAGGGGT TTGTGCCTCC AATGA (G/A) CACAAGCTCC |
| | CCCTGCCCCC CAACT |
| 5. | CCTGGCGGTG GCCGTCACCA GCTTT (T/C) GGGGGTGTTT |
| | GGGAAGCTGG |
| 6. | CTCCAGCCCC ACTGTTCCCT (A/G) GGCCCTATTG |
| | GTCCCCCTGG |
| 7. | ACAAGGAGGA GGCAGAAGTG AGGTT (G/C) AAACCCACTG |
| | CCCAATCTTA |
| 8. | CCAACACGGT GAAACCCCGT CTGTA (T/C) TAAAAATACA |
| | AAAATTAGCC |
| 9. | AATCCAGGAC CCCATAATCT TCCGT (C/T) ATCTAAAACA |
| | ATAATGGTGA |
| 10. | CCCAAGGGGG CGAGGGGAGG GTGAA (A/G) GGGTGGGACG |
| | GGGGCAGCCG |
| 11. | GAAGTGAGAA GGGGGCTGGG GGTCG (G/-) CGCTCGCTAG |
| | CGGGCGCGGG |
| 12. | CGCACGCGCA GTATCCCGAT TGGCT (C/G) TGCCCTAGCG |
| | GATTGACGGG |
| 13. | AACTCCTGGG TTCGATCAAT ACTCA (GACA/-) |
| | ATCTTGGCAG GCGCAGGAGG |
| 14. | GCTGGGATTA CAGGCTTGAG CCACC (A/G) CGCCCGGCCT |
| | GCAAAGCCAT |
| 15. | TTTTGTATCT TTAGTAGAGA CAGG (T/G) TTTCTCCATG |
| | TTGGTCAGGC |
| 16. | GCCTCAGCCT CCCGAGTAGC TGAGACT (C/A) CAGGTGCCCG |
| | CCACCACGCC |
| 17. | TGAAATTGTA GGTTGAGAGG CCAGGCG (C/T) GGTGCTCACG |
| | CCTGTAATTT |
| 18. | GTTTATAAAC ATTAAACCAG (T/A) GCTGTGTGAA |
| | GGCACTTAAT |
| 19. | CCGTCTCTAT TAAAAATATA AAA (A/C) AATTTAGCCG |
| | GGTGTAGCGG |
| 20. | GGGAGGCTCG AGGCGGGC (A/G) GATTGCATGA GCTCAGGATT |
| 21. | TCCCAAGTTT CAGGGCCCAA (T/G) ATTCTCAAAT |
| | CACAGGATTC |
| 22. | TGCAGTGAGC TGAGATCGC (A/G) CCACTGCACT |
| | CCAGCCTGGG |
| 23. | TCTTAGGACG CATGGGGGT (T/G) GAGAGAACGG |
| | GGAGATAGAC |
| 24. | CTGGGTTCTA GAACTACC (C/T) ATGCAAACCC AGCTGTTTCC |
| 25. | ATTCTGCCCT GGGTTCTAGA ACTACCT (C/A) TGCAAACCCA |
| | GCTGTTTCCC |
| 26. | GCTGTTTCCC ACCCCATAAG GCA (A/G) TAGGGGAGCC |
| | CACCTCCGCC |
| 27. | GACCTAGAAG ATCGGTCGAG A (C/T) AGCAGCTTGA |
| | GGCTGGCAGG |
| 28. | CTGGCCAGGA ATGCAGTCGG GTCAC (C/T) CTGTCTAGCC |
| | ACCGTCTCGC |
| 29. | GGGAGGAGTC GCCGATCAGG (C/T) CCCTTCCTGA |
| | AAGTCATCGA |
| 30. | GCAGCCCGGG CTACAGGGTT (A/G) CCTGAGGTGT |
| | GGGTCCCAGG |
| 31. | TAGAAATACT AACAAAGGGC (T/C) GTGGGTTTCT |
| | CCCCCTGCTT |
| 32. | ACAGGAGAGG GAAGGTTTTTTG (A/T) TTTTTTTTTT |
| | GTTTTTTTTT |
| 33. | GAAGAGGAAG AAGCCCAAAG GGA (A/C) AGAAACCTTC |
| | GAGCCAGAAG |
| 34. | GCGCCTCAAC AGCCAGAAGG AGCG (A/G) AGCCTCAGGC |
| | CCAGGCAGCT |
| 35. | TTGAGACTCT CTGTTTGAT (A/G) CTTCACTCAG |
| | AAGGTGCTTC |
| 36. | AGGCCAGGCT CCTGCTGGCT G (C/G) GCTGGTGCAG |
| | TCTCTGGGGA |
| 37. | CCCCTATACC CTCAAGCAT (C/T) TATCCATTGA |
| | GTTACAAACA |
| 38. | ACCATCCCCC GCCTTCCGTT (A/C) GTCCGGCCCC |
| | CGAGGCTAGC |

In another embodiment, the primers and/or probes are capable of hybridizing to a subsequence selected from the group of subsequences below:

| | |
|---|---|
| 1. | TGAAATTGTA GGTTGAGAGG CCAGGCG (C/T) GGTGCTCACG |
| | CCTGTAATTT |
| 2. | GTTTATAAAC ATTAAACCAG (T/A) GCTGTGTGAA |
| | GGCACTTAAT |
| 3. | CCGTCTCTAT TAAAAATATA AAA (A/C) AATTTAGCCG |
| | GGTGTAGCGG |
| 4. | GGGAGGCTCG AGGCGGGC (A/G) GATTGCATGA GCTCAGGATT |
| 5. | TCCCAAGTTT CAGGGCCCAA (T/G) ATTCTCAAAT |
| | CACAGGATTC |
| 6. | TGCAGTGAGC TGAGATCGC (A/G) CCACTGCACT |
| | CCAGCCTGGG |
| 7. | TCTTAGGACG CATGGGGGT (T/G) GAGAGAACGG |
| | GGAGATAGAC |
| 8. | CTGGGTTCTA GAACTACC (C/T) ATGCAAACCC AGCTGTTTCC |
| 9. | ATTCTGCCCT GGGTTCTAGA ACTACCT (C/A) TGCAAACCCA |
| | GCTGTTTCCC |
| 10. | GCTGTTTCCC ACCCCATAAG GCA (A/G) TAGGGGAGCC |
| | CACCTCCGCC |
| 11. | GACCTAGAAG ATCGGTCGAG A (C/T) AGCAGCTTGA |
| | GGCTGGCAGG |
| 12. | CTGGCCAGGA ATGCAGTCGG GTCAC (C/T) CTGTCTAGCC |
| | ACCGTCTCGC |
| 13. | GGGAGGAGTC GCCGATCAGG (C/T) CCCTTCCTGA |
| | AAGTCATCGA |
| 14. | GCAGCCCGGG CTACAGGGTT (A/G) CCTGAGGTGT |
| | GGGTCCCAGG |
| 15. | TAGAAATACT AACAAAGGGC (T/C) GTGGGTTTCT |
| | CCCCCTGCTT |
| 16. | ACAGGAGAGG GAAGGTTTTTTG (A/T) TTTTTTTTTT |
| | GTTTTTTTTT |
| 17. | GAAGAGGAAG AAGCCCAAAG GGA (A/C) AGAAACCTTC |
| | GAGCCAGAAG |
| 18. | GCGCCTCAAC AGCCAGAAGG AGCG (A/G) AGCCTCAGGC |
| | CCAGGCAGCT |

In yet another embodiment, the primers and/or probes are capable of hybridizing to a subsequence selected from the group of subsequences below

| | |
|---|---|
| 1. | GTTTATAAAC ATTAAACCAG (T/A) GCTGTGTGAA |
| | GGCACTTAAT |
| 2. | CCGTCTCTAT TAAAAATATA AAA (A/C) AATTTAGCCG |
| | GGTGTAGCGG |
| 3. | GGGAGGCTCG AGGCGGGC (A/G) GATTGCATGA GCTCAGGATT |
| 4. | TCCCAAGTTT CAGGGCCCAA (T/G) ATTCTCAAAT |
| | CACAGGATTC |
| 5. | TGCAGTGAGC TGAGATCGC (A/G) CCACTGCACT |
| | CCAGCCTGGG |

It is preferred in one embodiment that at least one sequence polymorphism is assessed in a region corresponding to SEQ ID NO: 1 position 1521-37752 (r), such as including at least one sequence polymorphism assessed in a region corresponding to SEQ ID NO: 1 position 7760-22885.

In another embodiment, the methods of the invention relates to at least one sequence polymorphism is assessed in a region corresponding to SEQ ID NO: 1 position 34391-37683, ending with the coding region of ASE-1 (cagcctgtgtag), where tag is the stop codon.

In another embodiment, the method of the invention relates to at least one sequence polymorphism assessed in a region corresponding to the S1 as shown in FIG. 4.

In another embodiment, the method of the invention relates to at least one sequence polymorphism assessed in a region corresponding to the S2 as shown in FIG. 4.

In another embodiment, the method of the invention relates to at least one sequence polymorphism assessed in a region corresponding to the S3 as shown in FIG. 4. More particular the method of the invention relates to at least one sequence polymorphism being a deletion assessed in a region corresponding to the S3 as shown in FIG. 4, more particular a 4 basepair deletion in a region corresponding to the S3 as shown in FIG. 4, even more particular a deletion of TGTC in S3 as shown in FIG. 4.

In a preferred embodiment the primers or probes are selected from one or more of the following:

| | |
|---|---|
| TGGCTAACACGGTGAAACC | (SEQ ID NO:7) |
| GGAATCCAAAGATTCTATGATGG | (SEQ ID NO:8) |
| GGGAGGCGGAGCTTGCAGTGA | (SEQ ID NO:9) |
| CTGAGATCGCACCACTGCAC | (SEQ ID NO:10) |
| GGTTTTCTGCTCTGCACACG | (SEQ ID NO:11) |
| CCTTTCTCCTTCCACCAACG | (SEQ ID NO:12) |
| CGGGCTACAGGGTTACCTGAG | (SEQ ID NO:13) |
| TCTGCAACCTGGTGCGAGCAGC | (SEQ ID NO:14) |
| CCTACCACCATCATCACATCC | (SEQ ID NO:15) |
| GCCTTGCCAAAAATCATAACC | (SEQ ID NO:16) |
| CCTCTCCCCAATTAAGTGCCTTCACACAGC | (SEQ ID NO:17) |
| AGCCAGGGAGGTTGAGGCT | (SEQ ID NO:18) |
| AGACAGCCCTGAATCAGCAC | (SEQ ID NO:19) |
| GCAATGAGCCGAGATAGAA | (SEQ ID NO:20) |
| TGGCTAGCCCATTACTCTA | (SEQ ID NO:21) |

According to another aspect of the present invention there is provided a diagnostic nucleic acid primer capable of detecting a r region polymorphism at one or more of positions in the r region as defined by the in SEQ ID NO: 1 or the s region as defined by SEQ ID NQ: 2.

The primer or probe may be a diagnostic nucleic acid primer defined as an allele specific primer, used, generally together with a constant primer, in an amplification reaction such as a PCR reaction, which provides the discrimination between alleles through selective amplification of one allele at a particular sequence position. The diagnostic primer is preferably 5-50 nucleotides, more preferably about 5-35 nucleotides, more preferably about 5-30 nucleotides, more preferably at least 9 nucleotides.

In accordance with the present invention diagnostic primers are provided, comprising the sequences set out below as well as derivatives thereof wherein about 6-8 of the nucleotides at the 3′ terminus are identical to the sequences given below and wherein up to 10, such as up to 8, 6, 4, 2, or 1 of the remaining nucleotides may be varied without significantly affecting the properties of the diagnostic primer. Conveniently, the sequence of the diagnostic primer is as written below.

Furthermore, as described above at least two sets of primer(s) and/or probe(s) may be combined in the method thereby increasing the correlation probability. This second or other set of primer(s) and/or probe(s) may be a nucleotide or nucleotide analogues hybridising to a region within the region r or to a sequence different from the region r. Said sequence different from the region r is preferably a region in chromosome 19, preferably in chromosome 19q. In particular such second or other primer or probe may be selected from one or more of the sequences below, or the complementary strands:

| | |
|---|---|
| GCCCCGTCCCAGGTA | (SEQ ID NO:74) |
| AGCCCCAAGACCCTTTCACT | (SEQ ID NO:22) |
| GTCCCATAGATAGGAGTGAAAG | (SEQ ID NO:23) |
| CCCTAGGACACAGGAGCACA | (SEQ ID NO:24) |
| TTGTGCTTTCTCTGTGTCCA | (SEQ ID NO:25) |
| TATCAGAAAAGGCTGGAGGA | (SEQ ID NO:26) |
| GAGTGGCTGGGGAGTAGGA | (SEQ ID NO:27) |
| GCCAAGCAGAAGAGACAAA | (SEQ ID NO:28) |
| CCTCAGATGTCCTCTGCTCA | (SEQ ID NO:29) |
| GCCACAGCCCCAGCAAGTAG | (SEQ ID NO:30) |
| AGGACCACAGGACACGCAGA | (SEQ ID NO:31) |
| CATAGAACAGTCCAGAACAC | (SEQ ID NO:32) |
| TTAGCTTGGCACGGCTGTCCAAGGA | (SEQ ID NO:33) |
| ACAGAATTCGCCCCGGCCTGGTACAC | (SEQ ID NO:34) |
| TTGAAACTGGAACTCTGAGAAGG | (SEQ ID NO:35) |
| TGGTGGATGGTGTGAAGCA | (SEQ ID NO:36) |
| CCTTTCTCCAACTTCTTCTCCATTTCCACC | (SEQ ID NO:37) |
| GGGGATCATGTCGTCAATGGACT | (SEQ ID NO:38) |
| ATGCCCTGTAGGTTCAATGG | (SEQ ID NO:39) |
| TGGAGGTCTTTAGGGGCTTG | (SEQ ID NO:40) |
| GGCTGGTCCCCGTCTTCTCCTTCC | (SEQ ID NO:41) |
| TCTCTGTTGCCACTTCAGCCTC | (SEQ ID NO:42) |
| GTCCTGCCCTCAGCAAAGAGAA | (SEQ ID NO:43) |
| TTCTCCTGCGATTAAAGGCTGT | (SEQ ID NO:44) |
| ATCCTGTCCCTACTGGCCATTC | (SEQ ID NO:45) |
| TGTGGACGTGACAGTGAGAAAT | (SEQ ID NO:46) |
| TGGAGTGCTATGGCACGATCTCT | (SEQ ID NO:47) |
| CCATGGGCATCAAATTCCTGGGA | (SEQ ID NO:48) |
| CACACCTGGCTCATTTTTGTAT | (SEQ ID NO:49) |
| TCATCCAGGTTGTAGATGCCA | (SEQ ID NO:50) |
| AGGCTCAACAAGGAAAAATGC | (SEQ ID NO:51) |
| GCTAGACAGTCAAGGAGGGACG | (SEQ ID NO:52) |
| AAAGGGTGGGTGTGGGAGACATTGG | (SEQ ID NO:53) |
| AAACCAACCTAGGCACCCCAAA | (SEQ ID NO:54) |
| CAGTGTCCAAAGAGCACC | (SEQ ID NO:55) |
| CTACCCCTTTAGCGACC | (SEQ ID NO:56) |
| TCCTGCCCCCAGAGCGTCACC | (SEQ ID NO:57) |
| GTACGGTCCACATAATTTTGGAGGA | (SEQ ID NO:58) |
| CGACGAACTTCTCTGAAGCGAA | (SEQ ID NO:59) |
| AGCGACACGGGCATCTGG | (SEQ ID NO:60) |
| ATGAGCGTCCACCTCCTGAACC | (SEQ ID NO:61) |
| AGGCAGCAGCATCGTCATCCCC | (SEQ ID NO:62) |
| TGCATAGCTAGGTCCTGC | (SEQ ID NO:63) |
| AACTGACRAAACTAGCTCTATGGGGTGGTGCCGCA | (SEQ ID NO:64) |
| CTGGCTCTGAAACTTACTAGCCC | (SEQ ID NO:65) |
| GCTGGACTGTCACCGCATG | (SEQ ID NO:66) |
| GGAGCAGGGTTGGCGTG | (SEQ ID NO:67) |
| TGCCCTCCCAGAGGTAAGGCCT | (SEQ ID NO:68) |
| CCCTCCCGGAGGTAAGGCCTC | (SEQ ID NO:69) |
| GATCAAAGAGACAGACGAGC | (SEQ ID NO:70) |
| GAAGCCCAGGAAATGC | (SEQ ID NO:71) |
| GGACGCCCACCTGGCCAACC | (SEQ ID NO:72) |
| CGTGCTGCCCAACGAAGTG | (SEQ ID NO:73) |

The primers and probes may be manufactured using any convenient method of synthesis. Examples of such methods may be found in standard textbooks, for example “Protocols for Oligonucleotides and Analogues; Synthesis and Properties,” Methods in Molecular Biology Series; Volume 20; Ed. Sudhir Agrawal, Humana ISBN: 0-89603-247-7; 1993; 1.sup.st Edition. If required the primer(s) and probe(s) may be labelled to facilitate detection.

Kit

According to another aspect of the present invention, there is provided a diagnostic kit comprising at least one diagnostic primer of the invention and/or at least one allele-specific oligonucleotide primer of the invention.

The diagnostic kits may comprise appropriate packaging and instructions for use in the methods of the invention. Such kits may further comprise appropriate buffer(s) and polymerase(s) such as thermostable polymerases, for example taq polymerase.

Preferred kits can comprise means for amplifying the relevant sequence such as primers, polymerase, deoxynucleotides, buffer, metal ions; and/or means for discriminating the polymorphism, such as one or a set of probes hybridising to the polymorphic site, a sequence reaction covering the polymorphic site, an enzyme or an antibody; and/or a secondary amplification system, such as enzyme-conjugated antibodies, or fluorescent antibodies. The kit-of-parts preferably also comprises a detection system, such as a fluorometer, a film, an enzyme reagent or another highly sensitive detection device.

The methods described herein may be performed, for example, by utilizing prepackaged diagnostic kits. The invention therefore also encompasses kits for detecting the presence of a polypeptide or nucleic acid of the invention in a biological sample (i.e., a test sample). Such kits can be used, e.g., to determine if a subject is suffering from or is at increased risk of developing a disorder associated with a disorder-causing allele, or aberrant expression or activity of a polypeptide of the invention. For example, the kit can comprise a labeled compound or agent capable of detecting the polypeptide or mRNA or DNA or RAI gene sequences, e.g., encoding the polypeptide in a biological sample. The kit can further comprise a means for determining the amount of the polypeptide or mRNA in the sample (e.g., an antibody which binds the polypeptide or an oligonucleotide probe which binds to DNA or mRNA encoding the polypeptide). Kits can also include instructions for observing that the tested subject is suffering from or is at risk of developing a disorder associated with aberrant expression of the polypeptide if the amount of the polypeptide or mRNA encoding the polypeptide is above or below a normal level, or if the DNA correlates with presence of an RAI allele that causes a disorder.

For antibody-based kits, the kit can comprise, for example: (1) a first antibody (e.g., attached to a solid support) which binds to a polypeptide of the invention; and, optionally, (2) a second, different antibody which binds to either the polypeptide or to the first antibody and is conjugated to a detectable agent.

Identification of an Allele as Having Implication for Risk of Cancer

An allele in the s or r region can be identified as correlated with an increased risk of developing cancer on the basis of statistical analyses of the incidence of a particular allele in two groups of individuals with and without cancer, respectively, according to the χ² test, which is well known in the art. Furthermore, an allele in the region can be identified as an allele correlated with prognosis of cancer on the basis of statistical analyses of the incidence of a particular allele in individuals demonstrating different prognostic characteristics.

Identification of Humans Having Increased Likelihood of Responding to Treatment

It is further contemplated that the present invention provides a method for identifying a human subject as having an increased likelihood of responding positively to a cancer treatment, comprising determining the presence in the subject of a s or r region allele genotype correlated with an increased likelihood of positive response to treatment, whereby the presence of the genotype identifies the subject as having an increased likelihood of responding to cancer treatment.

The treatment mentioned herein may be any cancer treatment, such as conventional cancer treatment, for example X-ray, chemotherapeutics, surgical excision or combinations thereof.

Protein Products of the Gene(s)

Gene products of the region s or r or peptide fragments thereof, can be prepared for a variety of uses. For example, such gene products, or peptide fragments thereof, can be used for the generation of antibodies, in diagnostic assays.

The gene products of the invention include, but are not limited to, human RAI gene products, and ASE-1 gene products. In the following the invention is described in relation to RAI gene product.

Gene product, sometimes referred to herein as an “protein” or “polypeptide”, includes those gene products encoded by the RAI gene sequences shown as position 7821-21350 in SEQ ID NO: 1. Among gene product variants are gene products comprising amino acid residues encoded by the polymorphisms. Such gene product variants also include a variant of the RAI gene product.

In addition, RAI gene products may include proteins that represent functionally equivalent gene products. In preferred embodiments, such functionally equivalent RAI gene products are naturally occurring gene products. Functionally equivalent RAI gene products also include gene products that retain at least one of the biological activities of the RAI gene products described above, and/or which are recognized by and bind to antibodies (polyclonal or monoclonal) directed against RAI gene products.

Antibodies to Gene Products

Described herein are methods for the production of antibodies capable of specifically recognizing one or more gene product epitopes or epitopes of conserved variants or peptide fragments of the gene products. Furthermore, antibodies that specifically recognize mutant forms are encompassed by the invention. The terms “specifically bind” and “specifically recognize” refer to antibodies that bind to RAI gene product epitopes at a higher affinity than they bind to non-RAI (e.g., random) epitopes.

Such antibodies may include, but are not limited to polyclonal antibodies, monoclonal antibodies (mAbs), humanized or chimeric antibodies, single chain antibodies, Fab fragments, F(ab′)₂ fragments, fragments produced by a Fab expression library, anti-idiotypic (anti-Id) antibodies, and epitope-binding fragments of any of the above, including the polyclonal and monoclonal antibodies described below. Such antibodies may be used, for example, in the detection of a gene product in an biological sample and may, therefore, be utilized as part of a diagnostic or prognostic technique whereby patients may be tested for abnormal levels of gene products, and/or for the presence of abnormal forms of such gene products. Such antibodies may also be utilized in conjunction with, for example, compound screening schemes, as described, below, for the evaluation of the effect of test compounds on gene product levels and/or activity.

For the production of antibodies against a gene product, various host animals may be immunized by injection with a RAI gene product, or a portion thereof. Such host animals may include, but are not limited to rabbits, mice, and rats, to name but a few. Various adjuvants may be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjuvants such as BCG (bacille Calmette-Guerin) and Corynebacterium parvum.

Polyclonal antibodies are heterogeneous populations of antibody molecules derived from the sera of animals immunized with an antigen, such as a gene product, or an antigenic functional derivative thereof. For the production of polyclonal antibodies, host animals such as those described above, may be immunized by injection with gene product supplemented with adjuvants as also described above.

Monoclonal antibodies, which are homogeneous populations of antibodies to a particular antigen, may be obtained by any technique that provides for the production of antibody molecules by continuous cell lines in culture. These include, but are not limited to, the hybridoma technique of Kohler and Milstein (1975, Nature 256:495-497; and U.S. Pat. No. 4,376,110), the human B-cell hybridoma technique (Kosbor et al., 1983, Immunology Today 4:72; Cole et al., 1983, Proc. Natl. Acad. Sci. U.S.A. 80:2026-2030), and the EBV-hybridoma technique (Cole et al., 1985, Monoclonal Antibodies And Cancer Therapy, Alan R. Liss, Inc., pp. 77-96). Such antibodies may be of any immunoglobulin class including IgG, IgM, IgE, IgA, IgD and any subclass thereof. The hybridoma producing the mAb of this invention may be cultivated in vitro or in vivo. Production of high titers of mAbs in vivo makes this the presently preferred method of production.

In addition, techniques developed for the production of “chimeric antibodies” (Morrison, et al., 1984, Proc. Natl. Acad. Sci., 81:6851-6855; Neuberger, et al., 1984, Nature 312:604-608; Takeda, et al., 1985, Nature, 314:452-454) by splicing the genes from a mouse antibody molecule of appropriate antigen specificity together with genes from a human antibody molecule of appropriate biological activity can be used. A chimeric antibody is a molecule in which different portions are derived from different animal species, such as those having a variable region derived from a murine mAb and a human immunoglobulin constant region. (See, e.g., Cabilly et al., U.S. Pat. No. 4,816,567; and Boss et al., U.S. Pat. No. 4,816,397, which are incorporated herein by reference in their entirety.)

In addition, techniques have been developed for the production of humanized antibodies. (See, e.g., Queen, U.S. Pat. No. 5,585,089, which is incorporated herein by reference in its entirety.) An immunoglobulin light or heavy chain variable region consists of a “framework” region interrupted by three hypervariable regions, referred to as complementarily determining regions (CDRs). The extent of the framework region and CDRs have been precisely defined (see, “Sequences of Proteins of Immunological Interest”, Kabat, E. et al., U.S. Department of Health and Human Services (1983)). Briefly, humanized antibodies are antibody molecules from non-human species having one or more CDRs from the non-human species and a framework region from a human immunoglobulin molecule.

Alternatively, techniques described for the production of single chain antibodies (U.S. Pat. No. 4,946,778; Bird, 1988, Science 242:423-426; Huston, et al., 1988, Proc. Natl. Acad. Sci. U.S.A. 85:5879-5883; and Ward, et al., 1989, Nature 334:544-546) can be adapted to produce single chain antibodies against gene products. Single chain antibodies are formed by linking the heavy and light chain fragments of the Fv region via an amino acid bridge, resulting in a single chain polypeptide.

Antibody fragments that recognize specific epitopes may be generated by known techniques. For example, such fragments include but are not limited to: the F(ab′)₂ fragments, which can be produced by pepsin digestion of the antibody molecule and the Fab fragments, which can be generated by reducing the disulfide bridges of the F(ab′)₂ fragments. Alternatively, Fab expression libraries may be constructed (Huse, et al., 1989, Science 246:1275-1281) to allow rapid and easy identification of monoclonal Fab fragments with the desired specificity.

Immunoassays for gene products, conserved variants, or peptide fragments thereof will typically comprise incubating a sample, such as a biological fluid, a tissue extract, freshly harvested cells, or lysates of cells in the presence of a detectably labeled antibody capable of identifying gene product, conserved variants or peptide fragments thereof, and detecting the bound antibody by any of a number of techniques well-known in the art.

The biological sample may be brought in contact with and immobilized onto a solid phase support or carrier, such as nitrocellulose, that is capable of immobilizing cells, cell particles or soluble proteins. The support may then be washed with suitable buffers followed by treatment with the detectably labeled gene product specific antibody. The solid phase support may then be washed with the buffer a second time to remove unbound antibody. The amount of bound label on the solid support may then be detected by conventional means.

By “solid phase support or carrier” is intended any support capable of binding an antigen or an antibody. Well-known supports or carriers include glass, polystyrene, polypropylene, polyethylene, dextran, nylon, amylases, natural and modified celluloses, polyacrylamides, gabbros, and magnetite. The nature of the carrier can be either soluble to some extent or insoluble for the purposes of the present invention. The support material may have virtually any possible structural configuration so long as the coupled molecule is capable of binding to an antigen or antibody. Thus, the support configuration may be spherical, as in a bead, or cylindrical, as in the inside surface of a test tube, or the external surface of a rod. Alternatively, the surface may be flat such as a sheet, test strip, etc. Preferred supports include polystyrene beads. Those skilled in the art will know many other suitable carriers for binding antibody or antigen, or will be able to ascertain the same by use of routine experimentation.

One of the ways in which the RAI gene product-specific antibody can be detectably labeled is by linking the same to an enzyme, malate dehydrogenase, staphylococcal nuclease, delta-5-steroid isomerase, yeast alcohol dehydrogenase, α-glycerophosphate, dehydrogenase, triose phosphate isomerase, horseradish peroxidase, alkaline phosphatase, asparaginase, glucose oxidase, β-galactosidase, ribonuclease, urease, catalase, glucose-6-phosphate dehydrogenase, glucoamylase and acetylcholinesterase. The detection can be accomplished by colorimetric methods that employ a chromogenic substrate for the enzyme. Detection may also be accomplished by visual comparison of the extent of enzymatic reaction of a substrate in comparison with similarly prepared standards.

Detection may also be accomplished using any of a variety of other immunoassays. For example, by radioactively labeling the antibodies or antibody fragments, by labeling the antibody with a fluorescent compound. Among the most commonly used fluorescent labeling compounds are fluorescein isothiocyanate, rhodamine, phycoerythrin, phycocyanin, allophycocyanin, o-phthaldehyde and fluorescamine.

The antibody can also be detectably labeled using fluorescence emitting metals such as ¹⁵²Eu, or others of the lanthanide series or by coupling it to a chemiluminescent compound.

Diseases

Described herein are various applications of gene sequences, gene products, including peptide fragments and fusion proteins thereof, and of antibodies directed against gene products and peptide fragments thereof. Such applications include, for example, prognostic and diagnostic evaluation of a disease, such as cancer, and the identification of subjects with a predisposition to such disorders, as described above.

The method according to the invention may be used in relation to any cancer form, such as, but not limited to, skin carcinoma including malignant melanoma, breast cancer, lung cancer, colon cancer and other cancers in the gastrointestinal tract, prostate cancer, lymphoma, leukemia, pancreas cancer, head and neck cancer, ovary cancer and other gynecological cancers. In particular the method is relevant for skin cancer, lung cancer, colon cancer and breast cancer, such as skin cancer and breast cancer, preferably wherein the skin cancer is basal cell carcinoma.

In particular, the method is relevant for early age cancer, such as early age breast cancer.

Gene nucleic acid sequences, described above, can be utilized for transferring recombinant nucleic acid sequences to cells and expressing said sequences in recipient cells. Such techniques can be used, for example, in marking cells or for the treatment of cancer. Such treatment can be in the form of gene replacement therapy. Specifically, one or more copies of a normal RAI gene or a portion of the RAI gene that directs the production of an RAI gene product exhibiting normal RAI gene function, may be inserted into the appropriate cells within a patient, using vectors that include, but are not limited to, adenovirus, adeno-associated virus, and retrovirus vectors, in addition to other particles that introduce DNA into cells, such as liposomes.

In another embodiment, the invention may be used in relation to inflammatory diseases, such as, but not limited thereto, rheumatoid arthritis, colitis ulcerosa, Crohn's disease, thyroiditis, neural inflammation as in Alzheimer's disease, and Guillain-Barré syndrome.

### EXAMPLES

The examples relate to prediction from sequence polymorphisms in the region s or r to cancer. Blood was collected before (example 6) or after (examples 1 through 5) the persons acquired cancer. However, the sampling time is considered immaterial, as DNA in a polyclonal blood sample is not expected to change over time.

The particular sequence polymorphisms analysed in these examples are listed in Table 6, together with their sources of information and their definition as sequences.

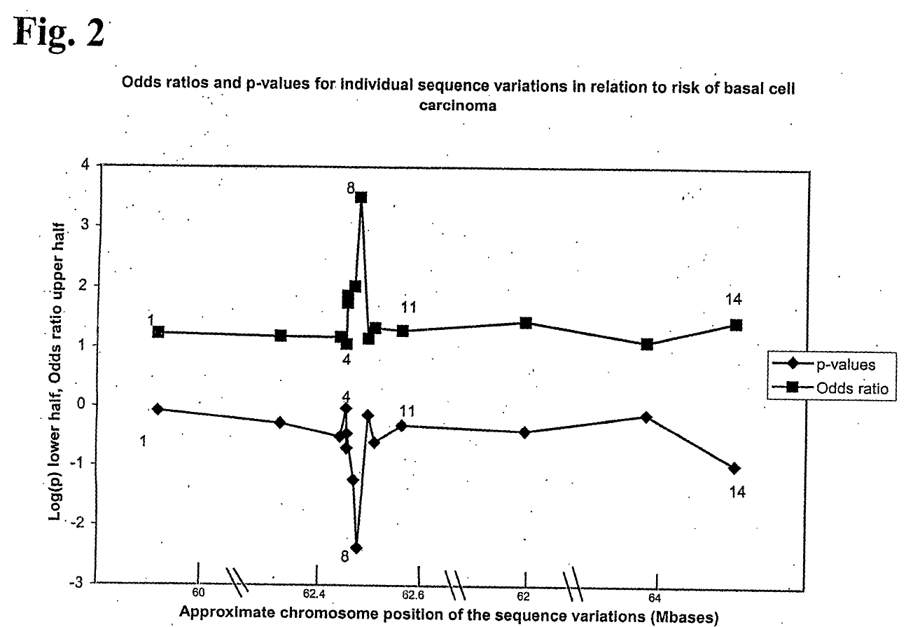

TABLE 6
The markers used, their sources of information, and their currently estimated
positions on chromosome 19, as well as their position in FIG. 2

| | | | | Chromosome | |
|---|---|---|---|---|---|
| | Source of | Position in | GenBank Accession | Position | Position |
| Name | identification | sequence | Number of sequence | (Mbases) | in FIG. 2 |
| XRCC1 e10 | Ref. 1 | 28152 | L34079 | 59.420 | 1 |
| CKM e8 | rs#8188 | 20076 | AC005781 | 61.361 | 2 |
| XPD e23 | Ref. 1 | 35931 | L47234 | 61.479 | 3 |
| XPD e10 | Ref. 1 | 23591 | L47234 | 61.491 | 4 |
| XPD e6 | Ref. 1 | 22541 | L47234 | 62.4923 | 5 |
| XPD i4 | rs#1618536 | 19244 | L47234 | 61.4924 | 6 |
| RAI e6 | rs#6966 | 8786 | L47234 | 61.506 | 7 |
| RAI i1 | rs#1970764 | 875 | L47234 | 61.514 | 8 |
| ASE1 e1 | rs#967591 | 232125 | NT_011242 | 61.534 | 9 |
| ERCC1 e4 | Ref. 1 | 19007 | M63796 | 61.547 | 10 |
| FOSB e4 | rs#1049698 | 34621 | M89651 | 61.601 | 11 |
| SLC1A5 e8 | rs#1060043 | 60620 | AC008622 | 62.946 | 12 |
| GLTSCR1 e1 | rs#1035938 | 20775 | AC010519 | 63.986 | 13 |
| LIG1 e6 | rs#20580 | 111 | L27710 | 65.460 | 14 |

rs numbers were derived from the NCBI's database dbSNP.
Ref 1: Shen, M. R., Jones, I. M., and Mohrenweiser, H. (1998) Nonconservative amino acid substitution variants exist at polymorphic frequency in DNA repair genes in healthy humans. Cancer Res., 58: 604-8, 1998.

Materials and Methods

Study groups. The groups of Caucasian Americans with and without basocellular carcinoma (BCC) have been described previously (Athas et al, Cancer Res. 51:5786-5793, 1991; Wei et al, Proc. Natl. Acad. Sci USA, 90: 1614-8, 1994). Briefly, the study was a clinic based case control study at the Johns Hopkins Hospital, which serves multiple participating dermatologists in Maryland. Cases were histo-pathologically confirmed primary BCCs and were diagnosed between 1987-1990. The controls were patients from the same physician practices and had a diagnosis of mild skin disorders. All participants were Caucasians living near Baltimore and were between 20 and 60 years of age. The controls were frequency matched to the cases by age and sex. Cases and controls with any other forms of cancer were excluded. In the questionnaire, the study subjects were asked if they had any blood relatives with skin cancer, and were asked to specify the type of cancer. Study subjects with relatives with basal cell carcinoma and squamous cell carcinoma and ‘skin cancer’ were included in the group of subjects with a family of skin cancer. Subjects with relatives with melanoma were not included. At the clinic visit the subjects gave informed consent, were examined by dermatologists, completed a structured questionnaire and provided blood. DNAs from available frozen lymphocytes were purified using Puregene (Gentra Systems) and were genotyped. Initially, 71 cases and 118 controls were included in this study. However, the number of persons varied between analyses, as the supply of DNAs was gradually depleted. In case of the SNP RAI i1 only 133 persons could be genotyped reliably.

The groups of 20 psoriatic Danes with and 20 psoriatic Danes without BCC have been described previously (Dybdahl et al, Cancer Epidemiol. Biomarkers Prev., 8:77-81, 1999). Briefly, BCC subjects were identified from a population-based cohort of persons treated by Danish dermatologists in the year 1995, and fulfilled the following criteria (a) age in 1995<50 years; and (b) clinically verified diagnosis of psoriasis. The diagnosis of BCC was clinically and histologically confirmed. The controls consisting of psoriasis cases without BCC was selected from among patients treated in the year 1992-1995 for psoriasis by dermatologists who participated in the national cohort study 1995. The controls were matched by age and sex. The patients with psoriasis and BCC differed from the national cohort of BCC in that the average of first BCC was 38 year against 56 year in the cohort. A number of cases had had multiple BCCs. There was a tendency that cases had been treated for a longer time than the controls, and also that the treatments were more intense. This was to be expected as treatment of psoriasis involves a number of carcinogenic treatment modalities. DNAs from available frozen lymphocytes were purified using Puregene (Gentra Systems) and were genotyped.

Primers and probes. Table 7 includes the polymorphisms typed on Lightcycler™, the primers used for the PCR reaction and the probes used for detection and typing of the PCR products. Table 8 lists the polymorphisms typed by conventional PCR-RFLP, and the primers and restriction enzymes used. Table 9 lists the polymorphisms typed by SNaPshot technology and the primers used. Table 10 lists the polymorphisms analyzed on a Taqman, and the primers and probes used. Hobolth DNA, Hillerød, Denmark or DNA Technology, Aarhus, Denmark, synthesized the primers in tables 7, 8, and 9. TIB Mol-Biol, Berlin, Germany, synthesized the Lightcycler probes. TAG-Copenhagen ApS (Tagc.com, Copenhagen, Denmark) synthesized the primers, and Applied Biosystem synthesized the fluorescent Taqman probes in table 10.

TABLE 7
Design of primers and fluorogenic probes for LightCycler

| | |
|---|---|
| ASE1 e1 | |
| Forward primer: | 5′-GGTTTTCTGCTCTGCACACG |
| Reverse primer: | 5′-CCTTTCTCCTTCCACCAACG |
| Anchor probe: | 5′-TCTGCAACCTGGTGCGAGCAGC-Fluorescein |
| Sensor probe: | 5′-LCRed640-CGGGCTACAGGGTTACCTGAG-p |
| CKM e8 | |
| Forward primer: | 5′-TTGAAACTGGAACTCTGAGAAGG |
| Reverse primer: | 5′-TGGTGGATGGTGTGAAGCA |
| Anchor probe: | 5′-LCRed640-CCTTTCTCCAACTTCTTCTCCATTTCCACC-p |
| Sensor probe: | 5′-GGGGATCATGTCGTCAATGGACT-Fluorescein |
| ERCC1 e4 | |
| Forward primer: | 5′-AGGACCACAGGACACGCAGA-3′ |
| Reverse primer: | 5′-CATAGAACAGTCCAGAACAC-3′ |
| Anchor probe: | 5′-LCRed640-TGGCGACGTAATTCCCGACTATGTGCTG p-3′ |
| Sensor probe: | 5′-CGCAACGTGCCCTGGGAAT-Fluorescein |
| FOSB e4 | |
| Forward primer: | 5′-AGGCTCAACAAGGAAAAATGC |
| Reverse primer: | 5′-GCTAGACAGTCAAGGAGGGACG |
| Anchor probe: | 5′-LCRed640-AAAGGGTGGGTGTGGGAGACATTGG-p |
| Sensor probe: | 5′-AAACCAACCTAGGCACCCCAAA-Fluorescein |
| GLTSCR1 e1 | |
| Forward primer: | 5′-CGACGAACTTCTCTGAAGCGAA |
| Reverse primer: | 5′-AGCGACACGGGCATCTGG |
| Anchor probe: | 5′-ATGAGCGTCCACCTCCTGAACC-fluorescein |
| Sensor probe: | 5′-LCRed640-AGGCAGCAGCATCGTCATCCCC-p |
| LIG1 e6 | |
| Forward primer: | 5′-ATGCCCTGTAGGTTCAATGG |
| Reverse primer: | 5′-TGGAGGTCTTTAGGGGCTTG |
| Anchor probe: | 5′-GGCTGGTCCCCGTCTTCTCCTTCC-Fluorescein |
| Sensor probe: | 5′-LCRed640-TCTCTGTTGCCACTTCAGCCTC-p |
| RAI i1 | |
| Forward primer: | 5′-TGGCTAACACGGTGAAACC |
| Reverse primer: | 5′-GGAATCCAAAGATTCTATGATGG |
| Anchor probe: | 5′-GGGAGGCGGAGCTTGCAGTGA-Fluorescein |
| Sensor probe: | 5′-LCRed640-CTGAGATCGCACCACTGCAC-p |
| SLC1A5 e8 | |
| Forward primer: | 5′-CAGTGTCCAAAGAGCACC |
| Reverse primer: | 5′-CTACCCCTTTAGCGACC |
| Anchor probe: | 5′-LCRed640-TCCTGCCCCCAGAGCGTCACC-p |
| Sensor, probe: | 5′-GTACGGTCCACATAATTTTGGAGGA-Fluorescein |
| XPD e10 | |
| Forward primer: | 5′-GATCAAAGAGACAGACGAGC |
| Reverse primer: | 5′-GAAGCCCAGGAAATGC |
| Anchor probe: | 5′-GGACGCCCACCTGGCCAACC-Fluorescein |
| Sensor probe: | 5′-LCRed640-CGTGCTGCCCAACGAAGTG-p |

TABLE 8

Primers and restriction enzymes used
for typing of SNPs using PCR-RFLP

| | | | |
|---|---|---|---|
| | | | Digested |
| Gene exon | Primers | Enzyme | Fragments |

| | | | | |
|---|---|---|---|---|
| XRCC1 exon10 | | TTGTGCTTTCTCTGTGTCCA | Mspl | 240, 375 bp (A) |
| | | TATCAGAAAAGGCTGGAGGA | | 615 bp (G) |
| ERCC1 exon4 | | AGGACCACAGGACACGCAGA | BsrDl | 157, 368 bp (A); |
| | | CATAGAACAGTCCAGAACAC | | 525 bp (G) |
| XPD exon6 | 1.set | CACACCTGGCTCATTTTTGTAT | Tfil | |
| | | TCATCCAGGTTGTAGATGCCA | | |
| | 2.set | TGGAGTGCTATGGCACGATCTCT | Tfil | 56, 114, 482 bp (A); |
| | | CCATGGGCATCAAATTCCTGGGA | | 56, 596 bp (C) |
| XPD exon23 | 1.set | GTCCTGCCCTCAGCAAAGAGAA | | |
| | | TTCTCCTGCGATTAAAGGCTGT | | |
| | | ATCCTGTCCCTACTGGCCATTC | Pstl | 66, 100, 158 (C); |
| | | TGTGAACGTGACAGTGAGAAAT | | 100, 224 (A) |

TABLE 9

Design of primers and SNaPshot primers
for SNaPshot typing on sequenator.

| | |
|---|---|
| XRCC1 exon7 | |
| Forward primer: | 5′-GTCCCATAGATAGGAGTGAAAG |
| Reverse primer: | 5′-CCCTAGGACACAGGAGCACA |
| SNaPshot primer: | 5′-TGCATAGCTAGGTCCTGC |
| XRCC1 exon17 | |
| Forward primer: | 5′-GCCAAGCAGAAGAGACAAA |
| Reverse primer: | 5′-GAGTGGCTGGGGAGTAGGA |
| SNaPshot primer: | 5′-AACTGACRAAACTAGCTCTATGGGGTGGTG |
| | CCGCA |
| RAI exon6 | |
| Forward primer: | 5′-CCTACCACCATCATCACATCC |
| Reverse primer: | 5′-GCCTTGCCAAAAATCATAACC |
| SNaPshot primer: | 5′-CCTCTCCCCAATTAAGTGCCTTCACACAGC |
| XPD intron4 | |
| Forward primer: | 5′-CGCAAAAACTTGTGTATTCACC |
| Reverse primer: | 5′-CCCATTTTTATCATCAGCAACC |
| SNaPshot primer: | 5′-CTGGCTCTGAAACTTACTAGCCC |

TABLE 10
Design of primers and probes for Taqman.

| | |
|---|---|
| XRCC1 exon10 | |
| Forward primer: | 5′-GCT GGA CTG TCA CCG CAT G |
| Reverse Primer: | 5′-GGA GCA GGG TTG GCG TG |
| Probe (A): | 5′Fam- TGC CCT CCC AGA GGT AAG GCC T -Tamra |
| Probe (G): | 5′Vic- CCC TCC CGG AGG TAA GGC CTC -Tamra |

Determination of polymorphisms by Lightcycler. Genotypes of the American persons for polymorphisms in ASE-1e1, CKMe8, ERCC1e4, FOSBe4, GLTSCR1e1, LIG1e6, RAIi1, SLC1A5e8 and XPDe10 and of the Danish persons for polymorphisms ASE-1e1, CKMe8, FOSBe4, LIG1e6 and SLC1A5e8 were detected using LightCycler™ (Roche Molecular Biochemicals, Mannheim, Germany). PCR was performed by rapid-cycling in a reaction volume of 20 μl with 0.5 μM of each primer, 0.045 μM of anchor and sensor probe, 3.5 mM MgCl₂, approximately 7-25 ng genomic DNA, and 2 μl LightCycler DNA Master Hybridization probe buffer (Roche Molecular Biochemicals, Cat. No 2158 825). This buffer contains Taq DNA polymerase, dNTP mix, and 10 mM MgCl₂ In some cases the reaction mixture also contained 5% DMSO. The temperature cycling consisted of denaturation at 95° C. for 2 sec, followed by 46 cycles consisting of 2 sec at 95° C., 10 sec at 57° C., and 30 sec at 72° C. The last annealing period at 72° C. was extended to 120 sec. The melting profile was determined by a temperature ramp from 50° C. to 95° C. with a rate of 0.1 degree/sec. For RAIi2 the melting profile was run 3 times, and the last curve was used.

PCR-RFLP analyses. Genotypes of the American persons for polymorphisms in XPDe6 and XPDe23 and of Danish psoriatics for polymorphisms in XRCC1e10, ERCC1e4, XPDe6, and XPDe23 were detected using PCR-RFLP technique (Shen et al see above; Dybdahl et al, see above; Vogel et al, Cancer Epidemiol. Biomarkers Prev., 8:77-81 (2001)). The reactions were performed as reported (Shen et al, see above; Dybdahl et al, see above; Vogel et al, Cancer Epidemiol. Biomarkers Prev., 8:77-81 (2001)).

Determination of polymorphisms by SNaPshot technique on sequenator. The polymorphisms in RAIe6, XPDi4 XRCC1e7, and XRCC1e17 in the American persons were typed simultaneously on an ABI Prism 310 sequenator (Applied Biosystems, Foster City, Calif., USA) using SNaPshot technique (Lindblad-Toh et al, Nature Genetics, 24: 381-6, 2000.). The PCR reaction consisted of 1 μl of purified genomic DNA, 1 pmole of each primer (DNA Technology, Aarhus Denmark), 12.5 nmole of each dNTP (Bioline, London, UK), 100 nmole MgCl₂ (Bioline), 0.15 μl BIOTAQ™ DNA Polymerase (Bioline) in a total volume of 20 μl of water. The program consisted of 4 min at 96° C., followed by 25 cycles of 96° C. for 30 sec, 60° C. for 30 sec, and 72° C. for 60 sec. The last cycle was followed by 72° C. for 6 min. The primers and dNTPs were removed in reactions containing 2 U Shrimp Alkaline Phosphatase (SAP) (Roche), 2 U Exonuclease I (Biolabs, Denmark), and 9 μl PCR reaction in a total volume of 14 μl water. The reactions were incubated at 37° C. for 60 min and 72° C. for 15 min. The SNaPshot reactions contained 1 μl of SNaPshot Ready Reaction Mix (Applied Biosystems), 0.5 μl of each SNaPshot primers (XRCCe7-ss1; 4 pmol/μl, XPDi5 cp1; 0.5 pmol/μl, RAIe7-cp1; 1 pmol/μl; XRCCe17-ss1; 2 pmol/μl), 2 μl of the purified PCR product, and 1.5 μl of buffer (200 mM Tris-HCl, 5 mM MgCl₂, pH 9.0). The reactions were cycled 25 times: 96° C. for 10 s, 50° C. for 5 s, and 60° C. for 30 s. The primers and dNTPs were removed in a reaction containing 1 U SAP, 0.8 μl 10×SAP buffer, and 5 μl SNaPshot reaction in a total volume of 8 μl of water. Two μl purified product was added to 10 μl of concentrated deionized formamide (Amresco, Ohio, USA), incubated for 5 min at 95° C., and analyzed on the sequenator. The two markers in XRCC1, in exon 7 and exon 17, could not be reliably scored and thus were excluded from further consideration.

Determination of polymorphisms by real-time PCR using Taqman probes. The polymorphism in XRCC1e10 in the American persons was analysed using the ABI Prism 7700 sequence detection system (Applied Biosystems, Foster City, Calif., USA). PCR Primers and Taqman probes were designed using Primer Express v 1.0 (Applied Biosystems). The reactions were performed in MicroAmp optical tubes sealed with MicroAmp optical caps (Applied Biosystems) containing a 10 μl reaction volume: 1×Taqman buffer A, 2.5 mM MgCl₂, 200 μM each of dATP dCTP, dGTP, 400 μM dUTP, 800 nM each primer, 200 nm each probe, 0.01 U/μL AmpErase UNG, 0.025 U/μL AmpliTaq Gold Polymerase. Thermal cycler conditions were: Tubes were incubated at 50° C. for 2 min followed 10 min at 95° C. The incubation was succeeded by 45 cycles of 95° C. for 15 sec and 64° C. for 1 min.

### Example 1

DNA from humans from the American cohort of patients with basal cell carcinoma and controls, described in Materials and Methods, was typed with respect to a number of sequence polymorphisms located in and around the claimed region r. The resulting statistical p-values for association of occurrence of the individual sequence polymorphisms with the status of patients are depicted in FIG. 2. Also depicted are the calculated odds ratios for association of sequence polymorphism and disease. For the calculation of the odds ratios the heterozygote genotypes were combined with the lesser group of homozygotes, and the ordering of the groups was chosen such that the odds ratio became more than or equal to 1. The results show that the sequence polymorphism RAIi1 is strongly associated with disease in this cohort (p=0.004). Bonferroni correction for the number of tests made indicates that a result less than 0.007 must be considered significant at a level of 0.05. Thus, even after correction for multiplicity of testing this result is significant.

The numbers next to the points in the curves are merely a help to identify the single sequence polymorphisms:

1, Xr1e10; 2, CKMe8; 3, XPDe23; 4, XPDe10; 5, XPDe6; 6, XPDi4; 7, RAIe6; 8, RAIi1; 9, ASE-1e3; 10, ERCC1e4; 11, FOSBe4; 12, SLC1A5e8; 13, GLTSCR1e1; 14, LIG1e6.

### Example 2

Those persons in Example 1 who got basal cell carcinoma before the age of 50 years were selected, and the results from analysis of RAIi1 were compared with the status of the patients. There was a strong relationship between the occurrence of the individual genotypes of the sequence polymorphism and the status of the patients (Table 11; Odds ratio=12.3; p(χ²)=0.00014).

TABLE 11
Occurrences of genotype for the sequence polymorphism
RAI i1 in American cases with Basal cell carcinoma occurring
before 50 years of age and in controls.

| RAIi1 | Number of cases | Number |
|---|---|---|
| genotypes | before 50 years of age | of controls |
| AA | 31 | 44 |
| AG | 2 | 32 |
| GG | 0 | 5 |

### Example 3

The data of Example 2 were combined with results of genotyping the neighbouring sequence polymorphism RAIe6. There was a very strong association between the combined genotypes of RAIi1 and RAIe6 and the status of the patients. Thus, almost all American cases occurring before the age of 50 yrs were homozygote for RAI i1^{A} RAI e6^{A}, while only approximately half of the controls were so (Table 12, Odds ratio=12.8; p(χ²)=0.00006).

TABLE 12
Combined occurrences of different genotypes for the sequence
polymorphisms RAIi2 and RAIe6 in American cases occurring
before 50 years of age and in controls.

| | | RAIi1 | | |
|---|---|---|---|---|
| RAIe6 | | AA | AG | GG |
| BCC casess | AA | 30 | 0 | 0 |
| | AT | 0 | 2 | 0 |
| | TT | 0 | 0 | 0 |
| Controls | AA | 42 | 10 | 1 |
| | AT | 2 | 21 | 0 |
| | TT | 1 | 0 | 2 |

### Example 4

The data of Example 2 were combined with results of genotyping the sequence polymorphism GLTSCR1e1 located outside the claimed region r. There was a very strong association between the combined genotypes of RAIi1 and GLTSCR1e1 and the status of the patients. It was obvious to define “risk-genotypes” as having two As in RAIi1 and at least one C in GLTSCR1e1. This corresponds to the assumptions that RAIi1^{A} is recessive, and GLTSCR1e1^{C} is dominant. If one does so, one finds that 25 out of 25 cases have a “risk-genotype”, while only 28 out of 62 controls have one (Table 13; Odds ratio>30; p(χ²)=0.000002).

TABLE 13
Combined occurrences of genotypes for the sequence polymorphisms
RAIi1 and GLTSCR1e1 in American cases of basal cell carcinoma
occurring before 50 years of age and in controls.

| | | RAIi1 | | |
|---|---|---|---|---|
| GLTSCR1e1 | | AA | AG | GG |
| BCC cases | CC | 17 | 0 | 0 |
| | CT | 8 | 0 | 0 |
| | TT | 0 | 0 | 0 |
| Controls | CC | 15 | 18 | 3 |
| | CT | 13 | 7 | 0 |
| | TT | 3 | 3 | 0 |

### Example 5

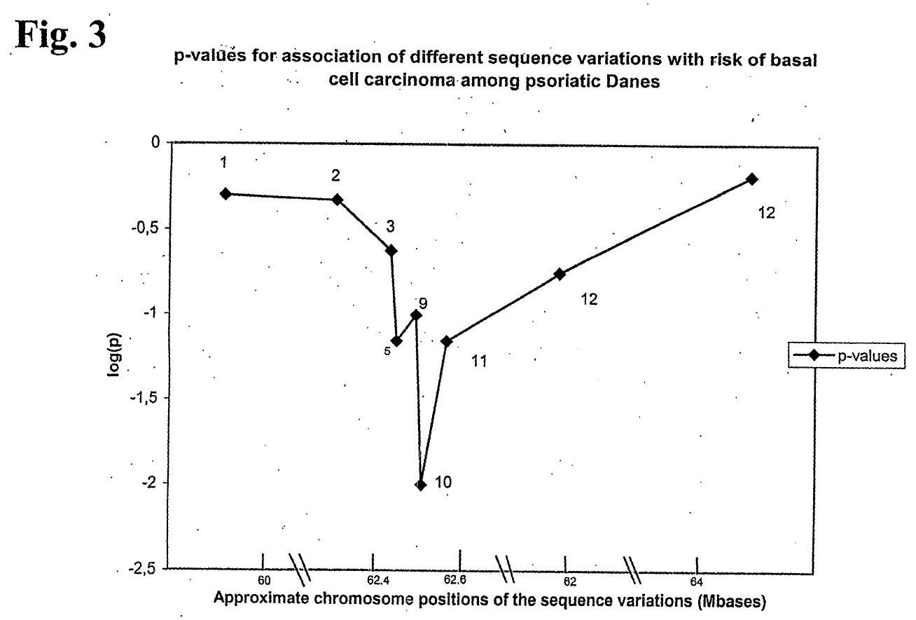

DNA from humans from the cohort of Danish psoriatics with basal cell carcinoma and controls, described in Materials and Methods, was typed with respect to a number of sequence polymorphisms located in and around the claimed region r. The resulting statistical p-values for association of occurrence of the individual sequence polymorphisms with the status of patients are depicted in FIG. 3. The results show that the sequence polymorphism ERCC1e4 is strongly associated with disease in this cohort (p=0.01).

### Example 6

Blood samples were collected from a large number of Danish citizens and frozen. After a number of years the women who got breast cancer in the intervening period were identified, as well as a set of matching controls. DNAs were purified from the blood samples of these persons and a number of polymorphisms, namely RAIi1, ASE-1e3 and ERCC1e4, in the region of interest were typed. The polymorphisms were subsequently combined such that the high-risk group was homozygous for the high-risk alleles of all three polymorphisms: RAIi1^{AA}ASE-1e3^{GG}ERCC1e4^{AA}. All other genotypes were combined into the low-risk group (Table 14; OR=1.59; p(χ²) 0.004).

TABLE 14
Occurrence of a combined “high-risk” genotype RAIi1^{AA}ASE-
1e3^{GG}ERCC1e4^{AA} as opposed to all other
combinations of genotypes for the sequence polymorphisms RAIi1,
ASE-e3 and ERCC1e4 in Danish cases of breast cancer and controls.

| | High-risk | Low-risk |
|---|---|---|
| Cases | 120 | 85 |
| Controls | 277 | 312 |

The DNAs in these examples were purified from available frozen lymphocytes using Puregene (Gentra Systems). A variety of other ways of purifying DNA is available to the expert and would also be expected to lead to the wanted results.

Analysis of sequence polymorphisms can be performed with a variety of techniques, some of which have been used in the examples of this application. Most often a number of techniques can produce the wanted result.

Similarly, the choice of primers and probes in a particular assay is to some extent free and other primers and probes might well produce similar results.

Finally, it is to be expected that assays for other sequence polymorphisms in the region of interest may produce roughly similar results. Our particular choice of sequence polymorphisms and assays used in the examples are thus not intended to limit our claims. Thus, at present about 30 SNPs within the region r are listed in NCBIs database dbSNP including rs#2070830, rs#2017104, rs#2017154 and rs#2377328, all within or very close to RAI. Other forms of polymorphisms such as the tandem repeat polymorphisms D19S543 and D19S393 are also known to occur in the region and can probably serve as markers in the present invention. Moreover, it is very likely that the region contains a number of as yet undiscovered polymorphisms. For instance, the sequence of the 5′ half of RAI and its upstream promoter region is currently only a draft version and new polymorphisms of potential use for this invention are likely to be uncovered as more sequence reads of this segment are produced.

Sequence of the r Region of Chromosome 19

The following depicts the region r stretching from the beginning of, but not including the XPD gene, to approximately the end of ERCC1, and includes the genes RAI, LOC162978, and ASE-1. More specifically r is bounded by and includes the following two sequences: AGAACCCCCG CCCCTCCACC TCGTCTCAAA and TCCCTCCCCA GAGACTGCAC CAGCGCAGCC, and is defined by SEQ ID NO: 1.

Sequence of the s Region of Chromosome 19

The following depicts the region s as described above. More specifically s is bounded by and includes the following two sequences:

and is defined by SEQ ID NO: 2

GGCGCCGGCCGGACTGTGCAG and CCAGAGACTGCACCAGCGCAGCC-
CAGCTTGAGCAAGATAGCG,.

### Example 7

The cases and controls in example 6 had been individually matched with respect to age, menopausal status and hormone treatment. Therefore, it was possible to make a paired analysis. This generally reduces the possibility of bias and confounding, but often produces less significant results. When the “high-risk” group was analysed, i.e. RAIi1^{AA} ASE-1e3^{GG} ERCC1^{AA}, versus all other genotypes, we found a rate ratio (RR)=1.64, Confidence Interval (CI)=1.17-2.29, and with a level of significance p=0.004. Thus, the “high-risk” genotype was clearly overrepresented among the breast cancers.

### Example 8

In the data of example 7, the “high-risk” group was further analysed, i.e. RAIi1^{AA} ASE-1e3^{GG} ERCC1^{AA}, versus all other genotypes, among those pairs that were less than 55 years of age. This increased the difference dramatically, indicating that the high-risk genotype predisposes to early breast cancer (rate ratio (RR)=9.5, Confidence Interval (CI)=2.2140.79, and with a level of significance (p)=0.003). In older age brackets, the RR was still above 1, but not significantly so. Thus, the combination of the three SNPs allows for the definition of a high-risk group for early breast cancer.

### Example 9

Blood samples were collected from a large number of Danish citizens and frozen (Example 6). The persons were also interviewed about a number of issues including smoking habits. After a number of years those persons, who got lung cancer in the intervening period, were identified, as well as a set of matched controls. DNAs were purified from the blood samples and a number of polymorphisms, namely XPDe10, XPDe23, RAIi1, ASE1e1 and ERCC1e4, in and around the region were typed. The three latter polymorphisms were combined into a “high-risk” group that was homozygous for the high-risk alleles of all three polymorphisms: RAIi1^{AA} ASE1e1^{GG} ERCC1e4^{AA}. All other genotypes at the three loci were combined into a low-risk group (Example 6). XPDe10, and XPDe23 were not combined with other markers. The results are shown in Table 15. It is clear that the “high-risk” genotype is associated with lung cancer in the youngest age group. XPDe23 shows signs of being associated at all age groups, while XPDe10 did not appear to relate to the disease. Therefore we recalculated the results for the youngest age group without XPDe10. Table 16 shows the results. Calculated this way both polymorphisms related to the risk of lung cancer.

TABLE 15
The risk of lung cancer in three different age groups in association with
the high-risk genotype, XPDe10, and XPDe23, mutually adjusted
for each other and for the duration of smoking.
High-risk genotype

| | High-risk | Rate Ratio | Confidence | |
|---|---|---|---|---|
| Age at diagnosis | genotype | (RR) | Interval (CI) | P-value |
| 50-55 | No | 1 | | |
| | Yes | 4.43 | (1.45-13.56) | 0.009 |
| 56-60 | No | 1 | | |
| | Yes | 0.73 | (0.30-1.83) | 0.51 |
| 61-70 | No | 1 | | |
| | Yes | 0.93 | | 0.82 |

XPDe10

| | | Rate Ratio | Confidence | P-value |
|---|---|---|---|---|
| Age at diagnosis | Genotype | (RR) | Interval (CI) | (trend) |
| 50-55 | GG | 1 | | 0.99 |
| | AG | 2.78 | (0.57-13.7) | |
| | AA | 1.2 | (0.14-10.4) | |
| 56-60 | GG | 1 | | 0.17 |
| | AG | 0.46 | (0.18-1.20) | — |
| | AA | 0.41 | (0.09-1.93) | |
| 61-70 | GG | 1 | | 0.40 |
| | AG | 0.91 | (0.46-1.80) | |
| | AA | 0.64 | (0.25-1.64) | |

XPDe23

| | | Rate Ratio | Confidence | P-value |
|---|---|---|---|---|
| Age at diagnosis | Genotype | (RR) | Interval (CI) | (trend) |
| 50-55 | AA | 1 | | 0.25 |
| | AC | 1.69 | (0.34-8.41) | |
| | CC | 3.62 | (0.39-33.6) | |
| 56-60 | AA | 1 | | 0.11 |
| | AC | 1.90 | (0.73-4.92) | |
| | CC | 3.40 | (0.71-16.3) | |
| 61-70 | AA | 1 | | 0.08 |
| | AC | 1.86 | (0.95-3.63) | |
| | CC | 2.23 | (0.79-6.31) | |

TABLE 16
Risk of lung cancer among those 50-55 years in association with
the high-risk genotype and XPDe23, mutually adjusted for each
other and for the duration of smoking.

| | | |
|---|---|---|
| Polymorphism | Rate Ratio (RR) | P-value |

High-risk group¹

| | | | |
|---|---|---|---|
| No | 1 | | |
| Yes | 4.27 | (1.42-12.89) | 0.01 |

XPDe23

| | | | |
|---|---|---|---|
| AA | 1 | | 0.01² |
| AC | 3.20 | (1.13-9.02) | |
| CC | 5.02 | (1.32-19.1) | |

¹RAIi1^{AA} ASE1e1^{GG} ERCC1e4^{AA}
²Trend test

### Example 10

In some of the samples of example 6 we typed a 4 bp deletion (dbSNP#3916791) located in the common portion of the sequences S1, S2 and S3 contiguous with sequence SEQ ID NO: 1. Specifically, the polymorphism is contained in the sequence GCGCCTGCCAAGATTGTCTGAGTATTGATCGAACCC, where the bases represented with boldface, italicised letters are present in some human chromosome 19 but not all. The deletion was typed by (1) Performing a PCR on the persons DNA with the primers 5′-6-FAM-TGAGACGAGGTGGAGG-3′ and 5′-CAATCAAAAAGAAAACATGG-3′. The fluoroscein-containing (6-FAM) primer was obtained from TIB-MOLBIOL (Berlin, Germany), while the other primer was obtained from DNA-Technology (Aarhus, Denmark). The reaction mix contained 0.84 U Taq polymerase (Roche), 1.7 nmole of each dNTP, 5 pmole of each primer, 1×PCR buffer (Roche), 1 M betain and approximately 20 ng DNA in a total volume of 9 ul. We used a temperature program containing 4 min denaturation at 94 C, followed by 30 cycles of 96 C for 1 min, 55 C for 30 sec, and 72 C for 45 sec; (2) We then mixed a sample containing 1 ul PCR product, 0.5 ul GeneScan-500 ROX size marker (Applied Biosystems) and 19 ul formamide; and (3) loaded the sample onto a single lane of Sequagel-6 matrix on a model 3100 Genetic Analyzer (ABI Prism, Applied Biosystems) using fluorescence detection. The persons who were homozygote for the complete fragment gave a length of 167 bp relative to the size markers, the persons who were homozygote for the 4 bp deletion gave a length of 163 bp, and the heterozygotes showed both lengths in roughly equimolar amounts. Because it has repeatedly been observed that the underlying risk-genotype seems recessive (Examples 2, 6, 7, 8), we pooled the homozygous low risk genotypes (163/163) and the heterogotes (163/167).

Table 17 shows the observed genotype frequencies among the cases and controls, the Odds Ratios for the genotypes, the confidence intervals, and the p-values for the Odds Ratios. Clearly, homozygosity for the 167 bp fragment was associated with increased risk of breast cancer.

TABLE 17
Risk of breast cancer in association with genotypes of the 4bp deletion in
S1.

| | Number of | Number of | Odds Ratio | Confidence | |
|---|---|---|---|---|---|
| Genotype | cases | controls | (OR) | Interval (CI) | P-value |
| 163/163 + 163/167 | 92 | 129 | 1 | | |
| 167/167 | 60 | 44 | 1.91 | (1.19-3.07) | 0.007 |

### Example 11

The blood samples described in Example 9 were analysed for the 4 bp deletion described in Example 10, and the results were combined with previous results for the polymorphism XPDe23. As a preliminary investigation showed the effects of the genotypes to be largely additive, the persons were grouped according to the number of “risk” alleles they were carrying, using the XPDe23^{AA} 4bp^{163/163} as the lowest risk, and thus placing those persons in group 0, and furthermore using them as reference for the calculation of the Odds Ratios. Table 18 shows the number of cases and controls in the different groups, the Odds Ratios for the different groups, the confidence intervals for the Odds Ratios and the p-values for the Odds ratios (calculated by the two-sided Fisher's exact test). Clearly, the risk of lung cancer increased dramatically with the number of risk-alleles.

TABLE 18
Risk of lung cancer according to the number of “risk”-alleles in the polymorphisms
4bp and XPDe23.

| Number of | Number of | Number of | Odds Ratio | Confidence | |
|---|---|---|---|---|---|
| “risk”-alleles | cases | controls | (OR) | Interval (CI) | P-value |
| 0¹ | 3 | 12 | 1 | | |
| 1² | 57 | 73 | 3.12 | (0.84-11.6) | 0.10 |
| 2³ | 123 | 129 | 3.81 | (1.05-13.8) | 0.034 |
| 3⁴ | 49 | 35 | 5.6 | (1.47-21.3) | 0.01 |
| 4⁵ | 4 | 1 | 16 | (1.27-200) | 0.03 |

¹XPDe23^{AA} 4bp^{163/163}
²XPDe23^{AC} 4bp^{163/163}, and XPDe23^{AA} 4bp^{163/167}
³XPDe23^{CC} 4bp^{163/163}, XPDe23^{AC} 4bp^{163/67}, and XPDe23^{AA} 4bp^{167/167}
⁴XPDe23^{CC} 4bp^{163/167}, and XPDe23^{AC} 4bp^{167/167}
⁵XPDe23^{CC} 4bp^{167/167}

### Example 12

The data of Examples 9 and 11 were combined and relative risks for lung cancer for the high-risk haplotype, the 4 bp deletion, and XPDe23 mutual adjusted for each other were calculated in 3 age-groups. The use of adjusted relative risks ensures that the effect of each marker is peculiar to it, and cannot be attributed any of the other markers in question. Tables 19, 20, and 21 show the result. After the adjustment it is apparent that all three markers have an effect independent of the others. Moreover, the adjusted effect of the high-risk haplotype is strongest among the youngest persons, while the adjusted effect of the 4 bp deletion is strongest in the oldest age group. XPDe23 exerts its adjusted effect at all ages, but possibly strongest in the youngest age group.

TABLE 19
Relative risks and 95 percent conficence intervals for lung cancer in
different age groups as a reflection of presence or absence
of the high-risk haplotype in homozygous form, adjusted for
the 4bp deletion and XPDe23.

| Age at | | | |
|---|---|---|---|
| diagnosis (YR) | Homozygous^{a} | RR | 95% CI |
| 50-55 | No | 1.00 | |
| | Yes | 4.26 | 1.38-13.17 |
| 56-60 | No | 1.00 | |
| | Yes | 1.07 | 0.36-2.98 |
| 61-70 | No | 1.00 | |
| | Yes | 0.82 | 0.44-1.53 |

^{a}Homozygous carriers of high-risk haplotype are defined as ERCC1 exon4^{AA}, ASE-1 exon1^{GG}, RAI intron^{AA}

TABLE 20
Relative risks and 95 percent confidence intervals and p-values for
trend for lung cancer in different age groups as a reflection of alleles at
the 4bp deletion site, adjusted for XPDe23 and the high-risk haplotype.

| Age at diagnosis (Yr) | Allele | RR | 95% CI | P(trend) |
|---|---|---|---|---|
| 50-55 | 163/163 | 1.00 | | 0.31 |
| | 163/167 | 1.35 | 0.36-5.02 | |
| | 167/167 | 0.35 | 0.11-2.87 | |
| 56-60 | 163/163 | 1.00 | | |
| | 163/167 | 1.76 | 0.58-5.38 | 0.75 |
| | 167/167 | 1.04 | 0.26-4.14 | |
| 61-70 | 163/163 | 1.00 | | 0.02 |
| | 163/167 | 0.67 | 0.36-1.22 | |
| | 167/167 | 0.36 | 0.16-0.82 | |

TABLE 21
Relative risks and 95 percent confidence intervals for lung cancer in
Different age groups as a reflection of alleles at the XPDe23 site,
adjusted for the high-risk haplotype and the 4bp deletion.

| Age at diagnosis (Yr) | Allele | RR | 95% CI |
|---|---|---|---|
| 50-55 | AA | 1.00 | |
| | AC | 3.13 | 0.95-10.33 |
| | CC | 7.86 | 1.78-34.64 |
| 56-60 | AA | 1.00 | |
| | AC | 1.33 | 0.60-2.95 |
| | CC | 1.95 | 0.63-6.06 |
| 61-70 | AA | 1.00 | |
| | AC | 1.81 | 1.07-3.07 |
| | CC | 2.54 | 1.16-5.56 |

### Example 13

The data of Example 9 concerning the high-risk haplotype were stratified according to age and gender and adjusted for smoking. The results are shown in table 22. It is obvious that most of the effect of the high-risk haplotype on risk of lung cancer is exerted on the young women, while the effect on men at best is very moderate.

TABLE 22
Sex and age group specific estimates of the lung cancer rate ratios
(RR) in association with the high-risk haplotype, adjusted for duration of
smoking.

| Age | Homozygous | Female | | Male | |
|---|---|---|---|---|---|
| group | for haplotype^{a} | RR (95% CI) | p | RR (95% CI) | p |
| 50-55 | No | 1.0 | | 1.0 | 0.75 |
| | Yes | 7.02 (1.88-26.18) | 0.004 | 0.80 (0.20-3.18) | |
| 56-60 | No | 1.0 | | 1.0 | 0.37 |
| | Yes | 1.03 (0.29-3.71) | 0.97 | 0.69 (0.30-1.58) | |
| 61-70 | No | 1.0 | 0.76 | 1.0 | 0.94 |
| | Yes | 0.89 (0.40-0.76) | | 1.03 (0.48-2.22) | |

^{a}Homozygous carriers of high-risk haplotype are defined as ERCC1 exon4^{AA}, ASE-1 exon1^{GG}, RAI intron^{AA}

1. A method for estimating the skin cancer, lung cancer, breast cancer and colon cancer risk of an individual comprising
assessing in the genetic material of a sample from said individual a sequence polymorphism
in a region corresponding to SEQ ID NO: 2, or a part thereof, or
in a region complementary to SEQ ID NO: 2, or a part thereof, or
in a transcription product from a sequence in a region corresponding to SEQ ID NO: 2, or a part thereof, or
or translation product from a sequence in a region corresponding to SEQ ID NO: 2, or a part thereof,
obtaining a sequence polymorphism response,
estimating the skin cancer, lung cancer, breast cancer and colon cancer risk of said individual based on the sequence polymorphism response.

2. The method according to claim 1, wherein a sequence polymorphism is assessed
in a region corresponding to SEQ ID NO: 1, or a part thereof, or
in a region complementary to SEQ ID NO: 1, or a part thereof, or
in a transcription product from a sequence in a region corresponding to SEQ ID NO: 1; or a part thereof, or
or translation product from a sequence in a region corresponding to SEQ ID NO: 1, or a part thereof.

3. The method according to claim 1, wherein the cell sample is a blood sample, a tissue sample, a sample of secretion, semen, ovum, a washing of a body surface, such as a buccal swap, a clipping of a body surface, including hairs and nails.

4. The method according to any of the preceding claims, wherein the cell is selected from white blood cells and tumor tissue.

5. The method according to any of the preceding claims, wherein the sequence polymorphism comprises at least one mutation base change.

6. The method according to any of the preceding claims, wherein the sequence polymorphism comprises at least two base changes.

7. The method according to any of the preceding claims, wherein the sequence polymorphism comprises at least one single nucleotide polymorphism.

8. The method according to any of the preceding claims, wherein the sequence polymorphism comprises at least two single nucleotide polymorphisms.

9. The method according to any of the preceding claims, wherein the sequence polymorphism comprises at least one tandem repeat polymorphism.

10. The method according to any of the preceding claims, wherein the sequence polymorphism comprises at least two tandem repeat polymorphisms.

11. The method according to any of the preceding claims, wherein the assessment is conducted by means of at least one nucleic acid primer or probe, such as a primer or probe of DNA, RNA or a nucleic acid analogue such as peptide nucleic acid (PNA) or locked nucleic acid (LNA).

12. The method according to claim 11, wherein the nucleotide primer or probe is capable of hybridising to a subsequence of the region corresponding to SEQ ID NO: 1, or a part thereof, or a region complementary to SEQ ID NO:1.

13. The method according to claim 11, wherein the primer or probe has a length of at least 9 nucleotide or peptide monomers.

14. The method according to any of the preceding claims 11-13, wherein at least one primer or probe is capable of hybridising to a subsequence selected from the group of subsequences

| | |
|---|---|
| 1. | GCTCTGAAAC TTACTAGCCC (A/G) GTATTTATGG |
| | AGAGGCATTT |
| 2. | GTGGTCAAAT TCTCATTCAT CGTGG (T/C) CCAGGCAAGC |
| | ACACTTCCTC |
| 3. | ACCCTGAGGT GAGCACCTGT TCCTT (C/T) TCCTTGCCCT |
| | TAGCCCAGAG GTAGA |
| 4. | GGGCAGGGGT TTGTGCCTCC AATGA (G/A) CACAAGCTCC |
| | CCCTGCCCCC CAACT |
| 5. | CCTGGCGGTG GCCGTCACCA GCTTT (T/C) GGGGGTGTTT |
| | GGGAAGCTGG |
| 6. | CTCCAGCCCC ACTGTTCCCT (A/G) GGCCCTATTG |
| | GTCCCCCTGG |
| 7. | ACAAGGAGGA GGCAGAAGTG AGGTT (G/C) AAACCCACTG |
| | CCCAATCTTA |
| 8. | CCAACACGGT GAAACCCCGT CTGTA (T/C) TAAAAATACA |
| | AAAATTAGCC |
| 9. | AATCCAGGAC CCCATAATCT TCCGT (C/T) ATCTAAAACA |
| | ATAATGGTGA |
| 10. | CCCAAGGGGG CGAGGGGAGG GTGAA (A/G) GGGTGGGACG |
| | GGGGCAGCCG |
| 11. | GAAGTGAGAA GGGGGCTGGG GGTCG (G/-) CGCTCGCTAG |
| | CGGGCGCGGG |
| 12. | CGCACGCGCA GTATCCCGAT TGGCT (C/G) TGCCCTAGCG |
| | GATTGACGGG |
| 13. | AACTCCTGGG TTCGATCAAT ACTCA (GACA/-) ATCTTGGCAG |
| | GCGCAGGAGG |
| 14. | GCTGGGATTA CAGGCTTGAG CCACC (A/G) CGCCCGGCCT |
| | GCAAAGCCAT |
| 15. | TTTTGTATCT TTAGTAGAGA CAGG (T/G) TTTCTCCATG |
| | TTGGTCAGGC |
| 16. | GCCTCAGCCT CCCGAGTAGC TGAGACT (C/A) CAGGTGCCCG |
| | CCACCACGCC |
| 17. | TGAAATTGTA GGTTGAGAGG CCAGGCG (C/T) GGTGCTCACG |
| | CCTGTAATTT |
| 18. | GTTTATAAAC ATTAAACCAG (T/A) GCTGTGTGAA |
| | GGCACTTAAT |
| 19. | CCGTCTCTAT TAAAAATATA AAA (A/C) AATTTAGCCG |
| | GGTGTAGCGG |
| 20. | GGGAGGCTCG AGGCGGGC (A/G) GATTGCATGA GCTCAGGATT |
| 21. | TCCCAAGTTT CAGGGCCCAA (T/G) ATTCTCAAAT |
| | CACAGGATTC |
| 22. | TGCAGTGAGC TGAGATCGC (A/G) CCACTGCACT |
| | CCAGCCTGGG |
| 23. | TCTTAGGACG CATGGGGGT (T/G) GAGAGAACGG |
| | GGAGATAGAC |
| 24. | CTGGGTTCTA GAACTACC (C/T) ATGCAAACCC AGCTGTTTCC |
| 25. | ATTCTGCCCT GGGTTCTAGA ACTACCT (C/A) TGCAAACCCA |
| | GCTGTTTCCC |
| 26. | GCTGTTTCCC ACCCCATAAG GCA (A/G) TAGGGGAGCC |
| | CACCTCCGCC |
| 27. | GACCTAGAAG ATCGGTCGAG A (C/T) AGCAGCTTGA |
| | GGCTGGCAGG |
| 28. | CTGGCCAGGA ATGCAGTCGG GTCAC (C/T) CTGTCTAGCC |
| | ACCGTCTCGC |
| 29. | GGGAGGAGTC GCCGATCAGG (C/T) CCCTTCCTGA |
| | AAGTCATCGA |
| 30. | GCAGCCCGGG CTACAGGGTT (A/G) CCTGAGGTGT |
| | GGGTCCCAGG |
| 31. | TAGAAATACT AACAAAGGGC (T/C) GTGGGTTTCT |
| | CCCCCTGCTT |
| 32. | ACAGGAGAGG GAAGGTTTTTTG (A/T) TTTTTTTTTT |
| | GTTTTTTTTT |
| 33. | GAAGAGGAAG AAGCCCAAAG GGA (A/C) AGAAACCTTC |
| | GAGCCAGAAG |
| 34. | GCGCCTCAAC AGCCAGAAGG AGCG (A/G) AGCCTCAGGC |
| | CCAGGCAGCT |
| 35. | TTGAGACTCT CTGTTTGAT (A/G) CTTCACTCAG |
| | AAGGTGCTTC |
| 36. | AGGCCAGGCT CCTGCTGGCT G (C/G) GCTGGTGCAG |
| | TCTCTGGGGA |
| 37. | CCCCTATACC CTCAAGCAT (C/T) TATCCATTGA |
| | GTTACAAACA |
| 38. | ACCATCCCCC GCCTTCCGTT (A/C) GTCCGGCCCC |
| | CGAGGCTAGC |

or to a sequence complementary to any of the subsequences.

15. The method according to claim 14, wherein at least one nucleotide probe is selected from the group consisting of

| | |
|---|---|
| 1. | TGAAATTGTA GGTTGAGAGG CCAGGCG (C/T) GGTGCTCACG |
| | CCTGTAATTT |
| 2. | GTTTATAAAC ATTAAACCAG (T/A) GCTGTGTGAA |
| | GGCACTTAAT |
| 3. | CCGTCTCTAT TAAAAATATA AAA (A/C) AATTTAGCCG |
| | GGTGTAGCGG |
| 4. | GGGAGGCTCG AGGCGGGC (A/G) GATTGCATGA GCTCAGGATT |
| 5. | TCCCAAGTTT CAGGGCCCAA (T/G) ATTCTCAAAT |
| | CACAGGATTC |
| 6. | TGCAGTGAGC TGAGATCGC (A/G) CCACTGCACT |
| | CCAGCCTGGG |
| 7. | TCTTAGGACG CATGGGGGT (T/G) GAGAGAACGG |
| | GGAGATAGAC |
| 8. | CTGGGTTCTA GAACTACC (C/T) ATGCAAACCC AGCTGTTTCC |
| 9. | ATTCTGCCCT GGGTTCTAGA ACTACCT (C/A) TGCAAACCCA |
| | GCTGTTTCCC |
| 10. | GCTGTTTCCC ACCCCATAAG GCA (A/G) TAGGGGAGCC |
| | CACCTCCGCC |
| 11. | GACCTAGAAG ATCGGTCGAG A (C/T) AGCAGCTTGA |
| | GGCTGGCAGG |
| 12. | CTGGCCAGGA ATGCAGTCGG GTCAC (C/T) CTGTCTAGCC |
| | ACCGTCTCGC |
| 13. | GGGAGGAGTC GCCGATCAGG (C/T) CCCTTCCTGA |
| | AAGTCATCGA |
| 14. | GCAGCCCGGG CTACAGGGTT (A/G) CCTGAGGTGT |
| | GGGTCCCAGG |
| 15. | TAGAAATACT AACAAAGGGC (T/C) GTGGGTTTCT |
| | CCCCCTGCTT |
| 16. | ACAGGAGAGG GAAGGTTTTTTG (A/T) TTTTTTTTTT |
| | GTTTTTTTTT |
| 17. | GAAGAGGAAG AAGCCCAAAG GGA (A/C) AGAAACCTTC |
| | GAGCCAGAAG |
| 18. | GCGCCTCAAC AGCCAGAAGG AGCG (A/G) AGCCTCAGGC |
| | CCAGGCAGCT |

or to a sequence complementary to any of the subsequences.

16. The method according to claim 15, wherein at least one nucleotide probe is selected from the group consisting of

| | |
|---|---|
| 1. | GTTTATAAAC ATTAAACCAG (T/A) GCTGTGTGAA |
| | GGCACTTAAT |
| 2. | CCGTCTCTAT TAAAAATATA AAA (A/C) AATTTAGCCG |
| | GGTGTAGCGG |
| 3. | GGGAGGCTCG AGGCGGGC (A/G) GATTGCATGA GCTCAGGATT |
| 4. | TCCCAAGTTT CAGGGCCCAA (T/G) ATTCTCAAAT |
| | CACAGGATTC |
| 5. | TGCAGTGAGC TGAGATCGC (A/G) CCACTGCACT CCAGCCTGGG |

or to a sequence complementary to any of the subsequences.

17. The method according to any of the preceding claims, wherein at least one sequence polymorphism is assessed in a region corresponding to SEQ ID NO: 1 position 1521-37752 (r).

18. The method according to any of the preceding claims, wherein at least one sequence polymorphism is assessed in a region corresponding to SEQ ID NO: 1 position 7760-22885 (RAI).

19. The method according to any of the preceding claims, wherein at least one sequence polymorphism is assessed in a region corresponding to SEQ ID NO: 1 position 34391-37752.

20. The method according to any of the preceding claims, wherein at least two different probes are used, one probe being selected from the probes as defined in any of claims 13-16, and the other probe being capable of hybridising to a sequence different from SEQ ID NO: 1, or a part thereof, or to a sequence complementary to a region different from SEQ ID NO: 1, or a part thereof.

21. The method according to claim 1, wherein the translational product from a sequence in a region corresponding to SEQ ID NO: 1, or a part thereof, is an antibody, such as a monoclonal or polyclonal antibody.

22. A method for estimating the disease prognosis of an individual comprising
assessing in the genetic material of a sample from said individual a sequence polymorphism
in a region corresponding to SEQ ID NO: 2, or a part thereof, or
in a region complementary to SEQ ID NO: 2, or a part thereof, or
in a transcription product from a sequence in a region corresponding to SEQ ID NO: 2, or a part thereof, or
or translation product from a sequence in a region corresponding to SEQ ID NO: 2, or a part thereof,
obtaining a sequence polymorphism response,
estimating the disease prognosis of said individual based on the sequence polymorphism response.

23. The method according to claim 22, wherein the method has any of the features as defined in any of the claims 2-21.

24. A method for estimating a treatment response of an individual suffering from cancer to a disease treatment, comprising
assessing in the genetic material of a sample from said individual a sequence polymorphism
in a region corresponding to SEQ ID NO: 1, or a part thereof, or
in a region complementary to SEQ ID NO: 1, or a part thereof, or
in a transcription product from a sequence in a region corresponding to SEQ ID NO: 1, or a part thereof, or
or translation product from a sequence in a region corresponding to SEQ ID NO: 1, or a part thereof.
obtaining a sequence polymorphism response,
estimating the individual's response to the disease treatment based on the sequence polymorphism response.

25. The method according to claim 24, wherein the method has any of the features as defined in any of the claims 2-21.

26. A primer or probe for detecting polymorphisms for use in a method as defined in any of the claims above, said primer or probe being selected from

| | |
|---|---|
| TGGCTAACACGGTGAAACC | (SEQ ID NO:7) |
| GGAATCGAAAGATTCTATGATGG | (SEQ ID NO:8) |
| GGGAGGCGGAGCTTGCAGTGA | (SEQ ID NO:9) |
| CTGAGATCGCACCACTGCAC | (SEQ ID NO:10) |
| GGTTTTCTGCTCTGCACACG | (SEQ ID NO:11) |
| CCTTTCTCCTTCCACCAACG | (SEQ ID NO:12) |
| CGGGCTACAGGGTTACCTGAG | (SEQ ID NO:13) |
| TCTGCAACCTGGTGCGAGCAGC | (SEQ ID NO:14) |
| CCTACCACCATCATCACATCC | (SEQ ID NO:15) |
| GCCTTGCCAAAAATCATAACC | (SEQ ID NO:16) |
| CCTCTCCCCAATTAAGTGCCTTCACACAGC | (SEQ ID NO:17) |
| AGCCAGGGAGGTTGAGGCT | (SEQ ID NO:18) |
| AGACAGCCCTGAATCAGCAC | (SEQ ID NO:19) |
| GCAATGAGCCGAGATAGAA | (SEQ ID NO:20) |
| TGGCTAGCCCATTACTCTA | (SEQ ID NO:21) |

27. The primer or probe according to claim 26, wherein the probe is operably linked to at least one label, such as operably linked to two different labels.

28. The probe according to claim 27, wherein the label is selected from TEX, TET, TAM, ROX, R6G, ORG, HEX, FLU, FAM, DABSYL, Cy7, Cy5, Cy3, BOFL, BOF, BO-X, BO-TRX, BO-TMR, JOE, 6JOE, VIC, 6FAM, LCRed840, LCRed705, TAMRA, Biotin, Digoxigenin, DuO-family, Daq-family.

29. The primer or probe according to any of claims 26-28, wherein the primer or probe is operably linked to a surface.

30. The primer or probe according to claim 29, wherein the surface is the surface of microbeads or a DNA chip.

31. A kit for use in a method as defined in any of the claims above, comprising at least one primer or probe, said probe being as defined in any of claims 26-30, and optionally further amplifying means for nucleic acid amplification.


SEQUENCE LISTING (172 sequences)

SEQ ID NO: 1 - DNA, 37790 bp, Human - part of chromosome 19
agaacccccgcccctccacctcgtctcaaaaaaaaaaaaaaatcgtctcagtagcgaatagtctaacggagaatgacagggaaattggtgatcctttctgggcccaagagttagaaatggctttgcaggccgggcgcggtggctcaagcctgtaatcccagcactttgggaggctgaggcaggtggatcacctgaggtcgggagttcaagaccagcctgaccaacatggagaaaacctgtctctactaaagatacaaaattagccgggcgtgctggcaaatgcttgtaatcccagctactcgggaggctgaagcaggagaattgcttgaacctgggaggcagaggttgcagtgagcagagatggcgccgtcgcactctagcctgggcaacaaaagcgaaactccatttcaaatattaataataataactaataaataaaacataaatgctagcttttgtttgtttcttcaacaaatagctatgtggcatctaccatgtgtctgatcctgtgctggcccctgggaacagaaaggtgaccatgacagcctcagcacctgccctcaaagaacagatttttttccttgagacagggtctttctctgtcgccaaggctggagtgcagtggcacagtcacagctcactgcagcctccacctcttgggctcaagcgatcctcccacctcagcttccagagtagctgggaccacaggtgtgcaccaccaagcccagctaagttttattttttaaatttttttagagacgaggtctcaccacgttgcccaggctggttaaactcgcaggttcaagtgatcctctcccctcagcctttcaaattgttgggattacaggggtgaggcaccaggcctggcctcaaagaacagatattaaatatacaaatgaatatatgattacagcctggagtggtggctcgtgcctgtggttccaacactttggaaggccaaggcgagtacattgcttgagctcaggagctagagaccagcctgggcaacatggtgaaaacccgtctctacaaaaaatgcaaaaattagctgggcgtggtggcgtgcacctgtagtcccagatactcaggaggctgaggtgggagaatcacctgggcctgggaggcagaggttgcaatgggcagtgattgtgccactgcactccagcctgggcaacaggagtgaaaacctatctcaaatgtgtgtgtgtgtgtgtgtgtgtgtgtgtgtgcgcacgtgtataatcacaagtacaaaagtgctgtgaaggaaaacttcaagtcaccataaagattgattatgggctgggtgcagtggctcatgcctgtaatcccagcactttgggaggccaaggcagatggatcacgaggtcaggagttcaagaccagcctggtcaacatggtgaaaccctatctctactaaaaaaaaaaaaaaaaaaaaaaaagccaggcatagtggcatgcatctgtaatcccatctactcgggaggctaaagcaggagaattgcttgaacccaggaggcagaagtgagccaagatcacgccactgcactccagcctgcgtgacagagcaagactccgtcccagaaaaagaaaaaaaaaaaagacttattatgacaggatgtctactgtcaactgtggggtgtgagtgttggccaagtgatcagagaaggcttcgtggaagaagcgaggtttgagtagagccagaaaataattagaagagatcaaccagcaagaggggatggatgagagaagtgagaaaggtgttccagggagagagaccatcatacacaaaagctctaggccagaagaaagctgaggcctgtgagtgctgaaaggaagcctgtgggggtggagctctgagttgagcacagggagcagagaaagggcagctggaggggaaggcaggggcagatcgaaatctcttttttaaattaattaattcttaatttatttatttttgagacaaggtctcactctttcgcccagactggagtacagtggcacaatctcagcgcaccgcaacctctgccacccaggctcaagcaattctctggcctcagcctccctagtagctgggattacaggtgcgcaccactactgcccagctaatttttatacttttagtagaaacggggtttcactatgttggccaggctggcctcaaactcctgacctcaaaagatccacccacttcagcctcccaaagtgctgggattacaggtgtgagccacccttcccggctgtatttttggagacagagtcttgctctgtcccagcctggagtatggtggtgtgaatttggctcattgccaccttgacctccagggctcaagtgatcctcccacctcagcctcctgagtagctgggactgcgggtacacgacaccacgcctggttaattttttttaattttttgtagagacgagggtatctcactatgttgtccaggctggttgaactcctgagctcaagcaattctcccacctcagcctcccaaagtggtgggattacagacgtgagccactgtgcccggcttaatttatttacataaatttttttatgtttacttttctatctcctacaggaagaaaatatattttgttattgacagggtctcgctatgttgcccaggctggtattgggctcaagccatcctgttccctcagcctcccaaagtactgggattacaagcgtgagcctctgcatccagcccagatccaaaatctttactgtcacctacagagtcctctgtaactagcttactgctcatcatccccataccaacccaccttactgctctgatctcctcctctctctcccccagctcattttgtttcagctatgctggtctccttgctgtctctaaaacataacaagcacatcccatctcagggcctttgcaccagctattttgtctgcctggaatgctgtttcccctgatagccatgtggctgacacactcacctccctcagctctttgctcaattgtcaacttctcggcccggcatggtggctcacacctgtaatcctaccactttgggaggctgaggtgggcagatcacctgagatcaggagttcgagaccagcctggccaagatggtgaaatcccgtctctactaaaaatacaaaaattggcaaagcatggtagcacataccagtaatcctagctacccgggaggctgaggcaggagaattgctggaacccgggaggcagaggctgcagtgagccaagatcatgccactgtactccagcctgggtgacaaagcaagactctgtctcaaaaaaaaaaaagtctccttctcaatgagggcttcctgaccaccaaattaaatctacctcctagacacacacacacacgcacgcacgcacgcacacacacacacgcacgcacgcacacacacacacacacacacactatatcccctttccctgctttattgttcttgagagctcatttaaccatgtgacatgctgaatattttacttatttattttgtttagaaagctcctggctgggcgcgggggctcacgcctgtaatcccagcactttgggaggctggaacaggtggatcatgtgaggtcaggagttccagaccagcctgaccaacacggtgaaacctcatctctattaaaaatgcaaaaattagctgggtgtggtgtcgcatgcctgtaatcccaactactcagaaggctgaagcaggagaatcgcttgaacctgggaggcagaggttaacgctgagccgagatcgcgccattgcactccagcctgggcaacaagagtgaaactctgtctcgaaaaaaacaaaagtcagctccatggcaggagtgatggctcacgcctataatcccagcactttgtgaggccgaggcgggcggatcacttgaggtcaggagttggagaccagcctggccaacatggtgaaacctcatctctactaaaaatacaaaaattagccgggcgtggtgacacatgtctgtagtcccagctacttgggaggctgaggctggagaatggcttgaacctgggaggtagaggttgcagtaagccaagatcgcgccattgctctccatcctgggcaacagactccgtctcagaaaggaagaaagaaggaaagagagaaagagagaaagagacagagagagagagagaaagggagaaagagagaaaggatggaaggaccctgacaagcactgttgcataaaagtttcttttctctctcttttttttttttttttttttttgagacagggtctcacttctgttgctccagctgaagtgcagtggtgagaacatggctcagtgcagcctcaacttcccaggcttaagtgatcctgccacctcagcctcctgagtagctgggactgtaggtgtgcaccaccgtgcctagctaattttttgtatttttagtagagacatggttccgccacgttgcccaggctggtcttgaactcctgggcttaagggatctgcccgccatggcctcccaaagtgctgggattaccagcgtgagccactgtacccagcctgagtataggtttctgataaattttaggatcatattgtttggactgggtaagaatttccagaactctaatgaagaaactgactggtttatattttattttattttattttattatttttgagatggattttcactcttgttgcccaagctggattgcagtggcacgatcttggctcaccacaacctccgcctcccggtttcaagtgattctcctgcctcagcctccccaggagctgggattacaggcacccaccaccatgctcggctatttttttttttatttttttatttttagtagagacggggtttcaccatgttggccaggctggtctcgaactcctgacctcaggtgatccacctgccttggcctcccaaagcgctgggattacaggcatgagccactgtgcaaggcctaggctggtttataaaattgctaaaccaagcagaacatgaattaaataccaaggaaatactctcctagattgtcatgttacatcagccaatactaaaattgtcaagatacacaatttgaatgaactccatggtccaagtcgaattatctatgatattacccatctaataaacagcactatgtcccttaatgggagaaaaagttggagaatttaagagaatatcaatccaatgttggttgggtgcagtgaatcatgtctatattcccagcactttgggaggccaaggcaggaggatcacttgagcccaggaattcaaggccagcctcggcaacacggtgagatcctgtctctacggaaaattaaaaaaaaaaaaagagagagattagtgggatgtggtgcctatagtcccagctacttgggaggctgaggcgggaggatcatttaagcctgggacgttgaggttgcagtgaaccatgagtgagactcatctcaaaaaaaaaaaaaaaatggcgatcactagaggaaaaaaaaactaaagtggggtttgcgggtagtgggagggcccttcctgctaggttgcactatgatctccagggaggctccacgggagaatcatttccttgtctttttcagtttctagagccaaattctttgcataccttgcattccttggctcggaaccccttccctaaccttcaaagctggcagctagcctctggctcaagtgtcacatggcctgtctctgtcttcctatccaatcttcctcttataagaacattggagccaggcatggtggctgacgcctgtaatcccagcactttgggagaccgaggcaggcggatcacaaggtcaggagttcgagaccagcctggccaacacagtgaaaccccgtctctactaaaaaaatacaaaaaagtagccgggcatggtggcaggtgcctgtaatcccagctacttgagaggctgaggcaggagaatcgcttgaacctgggaggcagagcttgcagtgagccgagatagtgccaatgcagtccggcctgggcgaaacagcgagactccgtcgcaaaaaaaaaaaaataataataaataataaataaaaataaaaataaaataaaaaaataaaaataataaaataaataaaaattattttgagacaaagtctattctgtggcagaggctggaatgcagtggcgtgatcacagcttactgcagcttctacctcctgagctcaagcgatccttccaccttggcttcctgagtagctgggacctcaggtgtacattaccacgctcagctaattatttatttatttattatatttttgtgacggagtttcgctcttgttgcccgggctggagtgcaatggtgctatctcagctcactgcaacctctgcctcctggattccagtgattctcctgtctcagcttcctgagtagctgggattacaggtacatgccatcacgcccagctaatttttgtatttttagtagagacggggtttcatcatattggtcaggctggtctcgaactcctgacctcaggtgatccacctgccttggcctcccaaagtgctgggattacaggcgtgaggcaccacgcccggcaattttttttttcttttttttttttcagacagagtcttgctctgtcacccaggctggagtgcagtagcgtgatctcggtttactgcaacctccatctcccgggttcaagcgattctcctttctcagcctcccaagtagctgggactacaggtgcacaccaccacggcgggctaatttttgtatttttagtagacaccaggtttcaccatattggtcagactggtctcaaactcctgacctcaggtgatccatctgcctcagcctcccaaattgctgggattacaagcgtgagccacacacctggcttaatttttttatttttgatcgacacagggtctccctatgttgtccaagctggcagagatttttgtttgtttgtttgagagggaattttgctcttgtagcccaggctggagtacaatggtgcaatcttggctcaccacaacttccgcctcccgggtttaacagattctcctgcctcagcctcccaagtagctggaactacaggcacctaccaccacaccaggctaatttttgtgctttttagtagagatgaggtttcaccatgttggccaggctggtcttaaactcctggcctccagtgatccacccgccttgacctcccaaagtgctgaaattacaggcgtgagcaccgcgcctggcctctcaacctacaatttcaacacccaaggaaacagcccaccatgagtgagaaccagcagacacaacaaactataggattagctgcctccaaacttcaggtgatagattatcaggcatgtacttgaaactaaaggacacaaaagaagaatccgaaatataaaataaaggattggacttgtgtgaaaagaatcccttagaaagggctactttcaggctggccatggtggctaatggcctgtaatcccagcactttggaaggccgaggtgtgtggatcacctgaggtcaagagttcaagaccagcctggccaacatggtgaaaccccgtctctactgaaaatacaaaaattagccaggtggggtggcagatgcctgtaatcccagctactcgggaggctgaggcaggagaatcgcttgaactcaggaggcagaggttgcagtgagctgagattgcgctatcgtgccccagcctgggcactagagtgagatcaaaaaaaaaaaaaaaaaaagaagaagaagaagaaagggctactttcagactgccttgccaaaaatcataaccacaatgatgagcatgtattgagtcaaaacagaatcaaaagagaagaaagtcaatttctgtgcaaactacttttatttataaggaaagtttctctattttgtttataaacattaaaccagtgctgtgtgaaggcacttaattggggagaggtggggcagggatcctggtagagaccaatgtttcccacccagaccccaagactgctgggagagatggtgtcagcagtgactcccaggaatatccagtggtgtggtggcccatcccaggcccggctgggcaggtggctggcttgctgggggatgtgatgatggtggtaggcatgggaggcactttggacgggatctgatttggcaaaaggaagtggtttcctgtccccagtgatttccagcccttcccagacctcccaaggctaaggcagattactaaatttaaggctggggccctccttcttccctggacttccaggagaacagagaaccggtggcaaggaccaccaccagcagggtgaggggtgcagataaaggcagcaaaaaacagagggagaggtctggagggaaggcaggaatgcttgtttctgtcagcctcagaaacctccttctatcctgctagactttactcctttgaggcttcaccctggggaacagctggggagagacaggatcttcagacatcaggagctcccacctcctcatcccacatgcaaatccgctgcctgtctctatcctcccaccccttcctaaggggacctctcagcacctcccaaactgctccagaatccaagttctgtgtcacctccaagaaccagatggaaccttccaatcagagcctccactgatgaaatggaatatttccagtgtctcctaactgccataaggagaagcccacctctctctaacaccttggttgtctttttgggtcccacctccatatttaaaaaatctcctctctcagggccgggagcagtgggtcacacctataatcccagcagtttgggaggccgaggtgggtggatgacctgagctcaggagttcaagacaagcctggtcaacatgacgagaccctgtctctactaaaaacacaaaaaattagctgggcgtggtggtgcatgcccgtaatcccagctacttgggaggctgaggcaggagaatcacttgaatccgggaggtggaggctgcagtgagccaagatcgcgccactgcactccagcctgggcgacgcagctgaagctgtgtctccaaaaacaaaacacacacacacacacacacagaaaaaaaaaaccaaaataaaaaaatctcccttctcaggaatgtaacggaatcttccttgccttctcccctaaccctaatagagaattttcctcagttacactgtaattttattaatggatttttcctcattctgcccaatgcagtgtaatgaaagcttcctctccatctgttatattatatataaatatatattatatatttatatattatatatttatatataacatataattttattgtcacccaggctggagtgcagtggcaccatcagggctcactgcaggatcaatctcccaggcttaagcgattctcctgtgtcagcctcctgatgagctgggattacaggcacccgccaccacacccggctaactttttttttttgtatttttagtagagatggagtttcaccatgttggccaggctggtctagaactcctgacctcaggagatccgcccgccttggcctcccaaagtgctgggattacaggtgtgagccacctggccgggccctccacttccttcttgtacattgctgaatccctgtgtcagccctagaggtccagtcttttgccctctcccagccttaatctacaattctgtaacccacccaccatcattaaaatgagattcttctttgtcgcttcccttggctaaaatggattattctttaacctctccaccaatacaaccagggatgataataaaaacattggattgagcagaaaccaatcaaataactagtaaggcagtactggcgagcaccctacatcctgacagctttataaagggcgcttccagccaggtgcggtggcacatgcctgtaatcccaggactttgggaggctgaggcgggcaggtcacctgaggtcaggagttcaagaccagcctggccaacgtgatgaaaccctgtctacacaaaatacaaaaaaaaaaaaaaaattagccgtgcgtggtggcatgcgcctgtcatcccagctactctggaggccaaggagggaggatcacttgagcccgggaggcagaggttgcagtgagcccacatcttatcactgcactccagtctgggtgacaaagcaagactccatctcaaataaataaatacaaattggccgggtgcggtggctcatgcctgtaatcccagcactttgggagaccaaggcaggtggatcatttgaggtcagtagatcaaaaccagcctggccaacatggtgaaaccccgtctctactaaaaatacaaaaagtagccgggcgtggtggtggtgggcgcctgtaatcccaggcaggagaactggttgagcccgggtggggggggcccgaggttgcagtgagcacagatggcgccattgcactccagcctgggcgacagagcgagactccgtttcagaaataaataaataaaataaaaataaaaataaaaaaataatagaaatttaaaaataaaataaagggcttttcctcacctactccactaactataagggacccttacccccgacattactattaaatataacggacttttcgtctcctccccatgagcaataatgagcttttcagacctccctctcccaatataacggtttgttcctgttgcctcttctttttcctgtgggatcccccttttccccaacccccaactgtcgggaggtccccatgacttctcccctgggctcaccccgaagtagttccgcggcacgtagccctcctggccgtgcagcgcggcccaccaccagtcggtctcctccggcccgtccctccgcagcacggtgaccgactcgccctcgcggaaggacagctcgtccccgaactcggcgctgtagtcccagagagcgtacactgccccgctgttcatcagccccatactctgctcgacgtctgaaacatgccacggaggggaaggtgagagcctggcccagggggtccaggaacaggggccacgtggggtccaggacagaccctggaatttggcgcctgtcccagcaaccacctgaaatgttgtgtgtgcccatggctgtggatgggaaccggagctggagtcagatgccgggactggccgtctttgagcgttcgaggaaactgggggaggcatgccagtgggccacccactcccgaggcagggtcagaggctcccatttcttttctttcttttttttttttttttgagacagagtctcgctctgtcgcccaggctggagtgcagtggcacgatctcggctcactgcaacctccgcctcccgggttcacaccattctcctgcctcagcctcccgagtagctgggactacaggcgcccgccaccacgcctggctaatttttggtatttttagtagagtcagggtttcaccgtgttagccaggatggtctcgatctcctgaccttgtgatccgcccacattggcctcccaaagtgctgggattacaggcgtgagccaccgcgcccggcctttttttttttttttttttttttgagatggaatttcgctcttgtcgcccaggcaggagtgcaatggtgcggtctcactgcaacctccgcctccggagttcgagccattctcctgcctcagccttccaagtagctgggattacaggtgtgcgccaccatgcctggccaatttttgtatctttagtagagacggggtttcaccatgttggtcaggctggtatcaaactcctgacctcaagtgatccacccgcctcggcctcccaaagtgctgggattacaggcgtgagccacctggcccggccctcatttccttcttgtacattgctgaatgcccgtgtcaaccctagaggtccagtcttttgccctaccctggcgcttagcttaagtggtacagtctctaaggaagattcgcaccttccttgaatgatagggtcctttaagttggctcatctgcctctttcttttcttttcttttcttttctttttggagacggagtcttgctctgtcgcccaggctggagtgcagtggcgcgatttcggctcactgcaacctccgcctcctgggttccagcaattctcctgcctcagcctccaaagtagctgggactacaggcccacgccgctacacccggctaaattgttttatatttttaatagagacggggtttcaccgtgttgcccaggctggtttggaaatcctgagctcatgcaatccgcccgcctcgagcctcccaaagtgctaggattacaggcatgagccaccgcgcctggctttctttttcttttcttttctttttttttttcagacaaggtctcactctgccacccaggctgcgggagtgcagtggtgagatcaagcttactgcagcctcgaacttccagattcaagcaatcctcctgcctcagcctcctcctgattctttatgttattattaaatattttgtaggccgggcacagtggctcacacctataatcacagcactttgggaggccaaggcaggcggatcctctgaggtcaggggtttgagaccagcctggccaacatggcaaaaccccgtctctactaaaaatacaaaaaaaaaaaaaaaaaaagttagcgggccgtggggcccttgcctgtaatcccagttactcgggagcctgaggcaggagaatcgctttcaccgaggaggcagaggttgtagtgggctatggtgccattgcactccagcctgggtgacagagcaagactctgtctcaaaaaataaataaataaaaataaataaatatttcgtagaggtcaggtgtggtggctcacacctgaatcttagcactttgggaggccaaggtgggcagattgcctgagctcaagagttcgggaccagcctgggcaacactgcaaaaccccttctgtactaaaaatacaaaaaaatgagtcgggcatggtggtgagcacctgtagtcccagctactcaagaggctgaggcagagaattgcttgaatccaggaggtggaggttgcagtgagccgagattgagccactgcactccagcctgggtgacagtgagactctgtctcaaaaataataataaataaatatttgtagagacagggggtctctacaatgtcttgtagcctgaccaggctcacctttcaaatatataaccctctgtctcacccataagtcctaggacctgcctcactccaactctccgtgaagttccttgcccacaccgagatacaactggctcctccaggtgtgaaatgaccctgtgcacaatccccgtggcacagcctacttcgccctgcccgtcggggaaccaggtgatgtagcctgccccctggagagatagggtacagccttgtgtcttcctacaagcccctttctggcagctgtagcctgctcacctgccagtggtgtggcaatgcctctcccacaagtggcagagcccacctgcccagagccctatgccaggtagatggcagggttgaaacgttcagctcctcacccttgaagatgtgaaaggtgagcagaccaatcttcacagccactctcctccccaaaggtgtccagctcgcatagcacagcctccatgtccccttttcccttaggagggcatagtccccccacccccgcaagcggtccatccctcatcctcctcctcggcaatcctgccaagtggttggtacagcccccatacccttctctccctagtagggggtagttgctcccctccccgctcctgcgcacccgccaggtacccaggcgccagcagccctgcctcgcacctgccaggtaggtggcgcagtcagcataaccctcgcggtaagggtcgcacttctcgaaggcggtggcgccgtcgctgagcgtggtggcgaagattgcagcgccgtgctgcaccagcgccatgcagatgactgtgtcgttgcacgacgccgcgcagtgcaagggtgtcctaggcgtgggggtggggggttgcggggaacgatgcgtgagaggctgcgcgtccgcccacgggggacccagcccaccgcgcgggtcggggctcaccagccgtggctgtcgggggagttgacattggcacccgcggtgatgaggaaatccacgatagagtagttggcgccgcagatggcgttgtgcaaggcagtgatgccctcctcgttgggctggctcgggtcgttcatctgagtgcaccgggggagggggaagactcagtcccgcggctggcatctgcgatgcccccgccgtgcccacctcccgctcagcagcgctcacctccttcaccgcctgctgcaccacctccagctccccggtcagcgccgcgtccaggaggagcaccagagggttgaggcgcgcgcggcgggccttgcgcggggagcccgccttccgcagcacagagcgcatctcctgggggacagggcgcagaggtcagcgacttggagggattgttagtatatccatgatctagagtaggaaacagaggtccagggacttgtggcacccatctagacaggggtagaactgggattccctcgggatggggtgagggggtgccttcgatctcctcctagagcctccagttccctgccatagacagggaatcctgtgatttgagaatcttgggccctgaaacttgggagaaagctggggggccatgggattggtggcaaagtaattctatcagttcaaaacaatgattgtggaagccagttatgcaattcacacacagtctcacatttcttttgttaataatgaatgcaatgagacacacatgacaaaatgttaccaggagtgttcattccggatgtttggaatttgagcattttattattccttgtattttccttttctttttctctttttttttttttttttgagatggagtctcgctctgtcacccaggctggagtgcagtgcagtggtgtgatctcagctcactgcaccctccatcccccaggttcaagcaattctcctgcctcagcctcctgagtagctaggattacaggcatgcgccactatgcctggctaattttcatatttttagtagagacagggttttgtcatgttgtccaggctggtctcgaactcctgacctcaggtgatccacccacctcagcctcccaaagtgctaggattacaggtgtgagccactgtgcccagcctcatgggctttcttatttttaattttcctcctgtaagattcatttattctgggctgggcgaggtggctcatgtctgtaatcctagcactttgggaggctgaggtgggaggatcacttgagcccaggagttcgagaacagcttgggcaatatagtgagacccagtctctacaaaaaataaaaaattagcctgacatggtggcgcacacccgtcgtcccagctacttgggaggctgaggcaggaggattacttgaatggaagagaaggaggcttcagtgagccatgatcatgccactgcactctagcctgggcaacagagtgagacccagtctcaaaagaaaaaaaaatgcatttatttattccaagtgtgtgagtgcatagcatttgtgattctggtctttgctgtttccagagtttcagtgattttaagattctggaattcagagatcccaacagccactgaattcaaaattcccagatgctcagttatttcaagtttccaatatgttgtgattgcagaaatgctaggctgtgctatttcaaattgctgaggggccaggactttggaatccaaagattctatgatggagaactttaatatttttctgttagaatttcttttttttgttggtttttttgagacagagtctcgctctgtcgcccaggctggagtgcagtggtgcgatctcagctcactgcaagctccgcctcccgggttcaggccattctcctgcctcagcctgccaagtagctgggactacgggcgcccgccaccacgcctggctattttgtatttttagtaaagatggggtttcaccgtgttagccaggaaggtcttgttctcctgacctcgtgatccgcccacctcggcctcccaaagtgctgggattacaggtgtgagccatcatgcctgacctagaatttcattttaaaagactagaaggaaatggctgggtgcggtggctcatgtgtgtaatctcagcactttgggaggctgaggagagtggatcacctgaggtcaggcaggagttcaagaccagcctggccaacgtggtgaaaccctgtctctactaaaaatacaaaaattaggtggccgtggtggtgcacgcctgtaatcccagctactcaggaggccgtggcatgagaatcacttgaacccaggaggcacagttatagtgagctgagatggcaccatcgcactccagcctgggtgacagagtgagactccatctcaaaaaaggaaaaaaaaaagaaagactagaaggaaatattcaaaatgttaatgatggttccctgtgagtggtgtgattttgtcctctttcttctatttttatttattttccccaagctctctatggtgttggtgtatttctctatagtggaatgtgtaaatttaaagtataaatctcagctgggcacagtggctcatgcctggtttgagaccagcctggacaacataatgagaactgtctctactgaaaatgttaaatattatctgggagtggtggtgcatgcctgtagtcccagccataggggaggctgaggcatgaggatcaattgagcccagtaggtggaggctgcagtgagccatgatcttgccactgcactccagcctgggcaacagagtgagactctgtctcgataataataaccctctattacaacatatcagtgcatgaatttgtgattttataattcaaaatatgagcatctttaattgtcagatttggtgacttcaagaatcagtaataatcagtctatgatactaactttataattattttttttaagagaagagtttccttttattttattttatttgagacagagtttctctctgttgcccaggctggagtgcagtggcgcaatctcggctcactgcagcctctgtctcctaggttcaagcaattctcctgcctgagcctcccgagtagctgggattacaggcatgcaccaccaggcccagctaatttttgtatttttagcagagacggggtttcaccatgttggcgaggctagtcttgaactcctgacctcaagtgatccacccgcctcggcctcccaaggtgctgggattacaggcatgagccaccgtgcccagcctaactttataattctaagatcgtgttcaaacctttaaatgctctagggctctaaaatgttactatcctaagacggtgacactagcgtttgattcttacattctatgattttttaagtttctctgtggccaggactctgtgattctacaatgggatgctcagccatttcaacatgttgttattcatcccctcttgatttcaaaatcctgagcctcaaggttccttgcctttactttcaggagggcctaggaataggcattttgggggggtccacctgacccctgcttctctgagaagtgatctcttcccgctgtctacgcacacggagtgttcaggactgttccatgtggctacaaccctcttcccagtcaagatgcagggaccaagatcagcaggagaccatcccctggtccaatggtgacaacagtaagagcagttaacagttatgtgccaggtattatgctaagcactacattaatgtatttaatcttggcggggtgtggtggctcacacctgtaatcccagcactttgggaggccagggcgggcagatcacttgaggtcaggagttcaagaccagcctagccaacacagtgaaaccccatctctactaaaaatacaaaaattagccaagcgtggtggcatatgcctgtaatcccagccacttgggagactgacgcaggagaatcactttaacccaggaggtggagtccagcacccagccgagactcacttgtttttatttatttatttatttatttttatttttattttttttgagacggaatcttgctctgtcacccaggctggagtgcagtggcgcgatctcagctcaccacaagctccgcctcccgggctcacgccattctcctctcagcctccagagtagctgggactacaggcgcccgccaccacccccagctaatttttgtatttttagtagagacggggtttcaccgtgttagccaggatggtcttatctcctgacttcgtgatccgcccgcctcggcctcccaaaatgctgggattacaggcatgaaccaccacgcccggcctatttatttatttatttagagatggagtcttgctctgtcgcccaggctggagtgcagtggtgcagtcttggctcactgcaacctccgccttccgggtttaagcgattctcttgcctcagcctcctgagtagctgggattggaatgagaccaccacttctcctgttgtccttcccagcttctcccccacctccccttttccctagtttataagacaggaaaaaaagggagaaagcaaaacgctggaaaaaaacagaagtacgataaatagctagatgaccttggcgccaccatctggtcctggtggttaaaataataataataatattaatccctgaccaaaactactggtgttatctgtaaattccagacattgtatgagaaagcactgtaaaacgttttgttctgttagctgatgtctgtagcccccagtcacgttcctcacgcttacttgatctatcgtggccctttcacgtggaccccttagcgttgtaagcccttaaaagtgctaggaatttctttttcggggagctcggctcttaagacgctgatgctcccggccgaataaaaacctcttccttctttaatccggtgtctgaggagttttgtctgtggctcgtcctgctacagaattacaggcacgcgccaccgctccgggctaatttttgtatttttttagtagacagggggtttcaccatgttggtcaggctggacttgaacctctgacctcatgatccacccacctcggcctcccaaagtgctgggattacaggcgtgagccaccgcgcccggccgagactcactattttataagaggagagagcaaagccaggaacagtggctcatgcctctaactgcagcaatttgggaggctgaggcaggtggatcatttgaagtcaggagtttgagaccagcctggccagcatggtgaaacctcatctctactaaaaatacaaaaattagccaggagtggtggcatacacttataatcccagctacttgggaagctaaagcgggaggatggcttgaacctgggaggcggaggttgcagtgagccgaggtcaagccactgcactccagcctgagtgatggagcaagactctgcctggaaaaaaaaaaaaaatagaggagagagcagagcagacacaagagacacagagacagagagggagagaagagagggtgactgctttgattcaggcaagacttctcagtcccagaatgaacccactgttgtgccaagactcagtcatgtccaggtgtatgactcgagattgctgaaggaatgcccggggcagggcacaggcacaggttattggagagaaggagcagagaacatctctatgtggccaagactcccagatggccctccatatagtcacacacagctatcctaaagactacatttcccagcatcccattgcaatgaggctcctggccagtgggagcaggcagagtgatgtatggaactcccaggttctgcctgaaacaggaaagggcactttctcttcttctttctctcttcctggctggagggcagacttggtgacagccatctaggaccatgaaggcaggcttactccccgatggatggcagagccccaggtagatagagcctgggtcctgactccagtgaggtgcctacagtcctgggctgcaaactcttggacttctactcaaaagaggagaaaacttcgatctcatctaagccactatatttggggggctctttgctacagctcctggattcatgtagcaaacataccccggtttcctcctgtattacttaccatgctctgcggctgctctggtgggctgctctgggacggggccgggggtggaatgggagctggtggggcaggagcagggggccctgccctggcctcagatccctcagtgatgggggacagctctggctccggccccccgggccctggccccccatgacgatggaagaggcggctgatgatctgctggtactgtttcttgtgggtagggggcagggccacagcaggggcctgctccatggagcccctgcgtttgaggggccggggaatttccgccaacacccgtgccacctcctccagctcgggcaccgactgtgcctccggtggcagtgctggctgcagcctcgtggggctgagaggccttgctacagggccttcatccacatcgccagcctccagcactggtgtcagcagcccctctatctccggctcaggctccagctcggtggggggtttggggggtcctagccggaacaagagcccatcagaggacaggtccccaggagacacccaacactccctctccacaacttccagggcatacaaccagcacatgattttctgtgtgacctcagggaagttccttgccctctctgggctacactttccttgggctgtgaataatatacaattatgatgcctcccatttattgagcagttagtatgtgcctggcgctttacatgcctaccttattgtaatctcaccactgctttgtgaggtagatacactgccatctccacattaccgaaagggaatctgggcctcagagaggacaagtcagttgcccaaagccatgcagttgggacttgaactcagttctggctgactctagaatctacttctaccaaccgtgatagatgtgattttctgagatcctgagagtttcctctcctaacatctcaggcagaaaactccagcaggaagtagaatcctggtgtttaatgatttcttctctgtcttactcattctgacagtaaagcaggtggaaataaaaatatgcattattggctgagtcgagtggctcacacctgtaatcccagaactttgggaggccgaggcaggcagatctcttgagatcaggagtttgagaccagcctggccaacatggtaaaaccctgtctctactaaaaatacaaaaaaaaaaaaaaaaaaaaaaaaattagctgggcgtggtggcacatgcctgtaatcccagctactcggaaggctgaggcacaggaatcgcttgaacccaggaggcggaggttgcagtgagccgagattgcaccactgcaccactgcactccagcctgggcaaaagagtgagatttcatctcaaaatatatatatatacacacacacacacaaacacacacacacattatatatatagtgtatatatatttttatatagtatgcatatacatataaataatacacacacacacacacggctgagcatggtggctcatgcctgtaatcccagcactttgggaggctgaggtgggtggatcacctgaggtcaggggttcgagaccagcctggccaacatggcaaaacctcatctctactaaaaacacaaaaaattagttgggtgtggtggtgcatgcctgtaaccccagctacttgggaagctgaggtaggagaatcgcttgaacctgggaggtgtaggatgcagtgagctgaaacctcaccactgcattccagcctgggcaagaagagtgaaactccatcttggctgggcacggtggttcacgcctgtaatcccagcactttgggaggccgaggtgggcagatcatgaggtcaggagatcgagaccatcctggctaacatgatgaaaccccgtctctactaaaaatacaaaaattagctgggggtggtggtgggcgcctgtagtcccagccactcgggaggctgaggcaggagaatggcgtgaacccgggaggcggagcttgcagtgagcaagcaccactgcactccaacctggaagaaagagcgagactctgtctcaaaaaaaaagagtgaaactctgtctcaaaaataaataaataaataaaccccaaaacacacacacatacacattatttcattgaatccccgtcacaattctatagggtagatattattaatctctcttcacagacgggaaacagagtttcggacaagtaatttatcttcagtcacacagcaagttagcagtgaagagagactccagcccatctgcttaactcactgatctcacacctcaaaatattaataaattattataactaatatggtagctatttatttgagactgggtctcactctgtcacccaggctggagtgcagtggcgctatcacagctcactgcagcctggatctcccaggcttaaatgatcctcccacctcagcatcctgagtagctgggactacaggcgcccactaccatgcccggcagattttttgtacttttatttttagtaaagtctattttagtttcactatgttgcccaggctggtcttgaactccagagctcaagcaatcctgtctgcattagcccaccaaactgctaggattacaagggtgagccacggtgcctggctaatatggtagctattgatagcttactatgtatcagatcctatttatttatttatttttgagacagagtctcaccctgtcacctgtgctggagtgcagtggcatgatcttggctcactgccacctccgcctccttggctcaagctgagtagctaggactacagtggtgagccaccatgcccagctaatttttttttttttttttttttttgatagagatgggatttcatcatgttgtccaggctggtcttgaactcctgacctcaagtgatctgcccacctcggcctcccaaagtgctgggattacaggtgtgagcaactgcacctggcccatcaggtgctgttttaaaggctttatatgaatttaataacatatgtcaataggatcgattctatcattatttgcctttttttttttttttttttttgaggcagagtctccccgtcacccaggatggactgcagtggcgcaatctcggctcactgcaacctccacctcccgggtccaagtgattctcctgcctcagcctcccaagtagctgggactacaggcgcccgccaccatgcctggctaatttttgtatttttagtagagatggggtttcatattggccaggctggtctcgaacttctgactttgtgatccgcccgcctcggcctcccaaagtgctgggattacaggcatgagccaccgtgcccggcccattatttcccttttacactcaagaaaattgaggcccagtgaggttaagtgacttgcccaaggtcacacagcgtggaaccaggcagtctggcttcagggtccacacttaacctttgagctatccctggctcctacccaaattcccaaactcacctggcctagctctctgcagggacagtgcttgtaaagaggcatttggctgtgatctccccacctcccagggctggtctggtccccctgccatttgtcctcccttcacccagtcctctagggccctcattgctgactcaccttcgttcacaggggccatgtctgttggggatgctggggggctggggtaggggtttggggttgggtctggggctgtgggggcagctggggctgtggttgtgattgtggctggggctgtggttgtggttggggctgcagcttaggcgggggtgctcgggtgaagaggggggacccagggagcatggcgcggctggccccgtgctcccagaaggcgttctgcagcttgaagatcatgctgagggggatgggacgctggcgcggggccccgcggggctgggggctggaggggggcatggggatgcggctgacgggctgccagctgcgaggcaaagtgcccgacggccccgcggagcccagcgagcgccggtagctgcccgcgtctgaacgccggtcgctggccagaggagagaccttgtaattgcgcggcagggtggcgctagtgaggttgtcctggggaagagggaagggagaaggggatcgggtgagagagggaaggtggaggggaggtaaagacaaaagacgagaagggagaggaggtgagggaagccctgggagtgagggagaagaaagggtgaggaaggagcagaaacccagcacagtgaagggagagcgtgggaacgggcgccgagacccagatcgcagccccgagggggagactggccttgaccccgctcccccaccccactcctcgaccttccccagcctctcctccccaggcgtcgcctcctcaccttgccggtgccccccagtccatccaggctgctctccctccaaggcaacagctgcaggctcggcgaggcaggccttgcgaagacgtccaggcctgcggggcgggaatcattagggtctgtggggctgcctctcctccgggtcctccattccccgggcctccaccactcacgttcatagctcgctgtctgcgaaggcttcttctcgtacgccacgtccaggtcagactcgttccaggctttcggaggccgccggcgcagcgtcaggtcgtctggggagaagtttccagggaggatgagacgggaggggtggcgagccccggatcctgcccgctttgaccccgcgagtcaaaggccccgcgaggggcccctgggttcaccttgcgcgcgcagaggcggggcgaatgcgctgccgccggagcctagcagggagctcccgaaggcggacgctggcgcgtcgtaggctgtggcaggggggcgcggtgacggcccacgctcggggaagaaggcctggggcccctccgccagggggctgccgcggggggagcctgcgcggcccaggaagtcgaaaggcgtggggggaccctgctggcggagcgggcctggcccgggccgcggggagggcgcacggccgagggagctgcctgcgccatcgaaggcgcggggccggggcgaggtcgcgcggtccaggctgccgtaggcgtccggctgcaggtagagcggggtgcgcggcgacgacggccgtcccttgggggacagcgggctgtaggggtgtagggttggggcactctctgatcgtccgaacggggtgtctgcgccgtcggtggccgccttccggggggaccctcggctgccgaagggctcagggatcgagctggagctgtaccggggcggctgtggggaggccagggcattgagggatggatcaaaggagacattagtggaagggttggtgtgtgggcgggggtgtcaagagagatcactggaggtcaacccagaggaggctgaccggccatggaaattcaggcacagagagcccaggtgagtagtggtggggagacagccctgaatcagcactgtggctagcccattactctatgtcacctttatgccacttaggtaaacacctctttccttctgagggtccctttagatgtccacttccactggtcccctcttttctatttctttctttctttctttctctctctttcttttctttctttctttcctctctctccttccttcctttctctctctctccttccctccctccctccctccctgcttgcttgctttctctctctctctttctttctttctttctttctttctttctttctttctttcttttctatctcggctcattgcagcctcaacctccctggcttagtgtgatcctcccacttcagcctcccaagtagctgggattacaggtatgcaccaccacacctggctaacttttgtatttttagtagagacagggtttcaccatgttagccaggctggtcttaaactcctgacctcaagtgatccgcctgtctctgaaagtgttgagattacaggcgtgaaccaccgtgcccagccagatttttaaaaaatcatttgtagaggctggtctcaaactcttagtctcaagcaattctctcacctcgccttccaaagtgctgggattccaggtctgagccatcgcgcctggcctggtccccttttttcaagttcccttgaagagcccacaacctgcataactatatggggcaattttgcctgaaatccaggcctctggtctggactgtggcgagaggctggctttggagatcaaggtgggaaccaggcttaccctagaagggggtccggcctgcgggccaggaggcgcgggagagtctgaccacagcgactccagctgcttggtcagttcatccaccttggccgccgccgtgtccagctccatctgcttcagatccatgtgtttcatggccagcgctgggaaggtgggagtggaggtaaggacctggcctcctggcaggggccggcctcagcacccctcgcccgctgccgaggtccccgcctcgccagccccgccccctactccagcttacactggaagttcatgtccagaaagtcccgcgcgctctggaatgcctcgctgtccatggtgccggccggagcgggcgcctgcatggtggggagggagggagctggctaagaccccgcccctctagaccccgccctcagggagtcagacgccgtcaggagcgggacaacgcctcaactcagttccttcccctggaagccctttaccctttcacctccccagctgggaaatgccaactcctccaaagccaagtccatgcgccacggagaagtccaaacccagtctaaaacctccggaattcactttctctttctttttttcttttctttttttttttttttttgtgtatgtgtgtgagacagagtctcgctctgtcgcccaggcgggagtgcaatgacgcgatcttggctcactgcaacctccgcctcccgggttcaagcaaatcttctgcctagctgggactacaagcgcgcgccattatgcccggctaatttttgtagttctgggattacaggagtgagtctccgcgcccggccgtgtccatctctttatctcagtcctaagacctgaatcactccttgaacaattatctattgatcacctacaatgtgccggtaaacataggatggaataactatgaattactgaatgtttactagggaccaggacgcactgtgctagatcctgtttttgtttgtttttgagatggtgtctcgcattttcgcccaggctggagtgcagtggcgcgatctcggctcactgcaagctccgcctccagggttcatgccagtctcctgtctcagcctcccgagtagctgggactacaggcgcctgccaccatgcctggctaaatttttgtatttttagtagagacggggtttcaccgtgtcagccaggatggtctcgatctcctgaccgcgtgatccatctgcctcggcctcccaaagtgctgggattacaggcgtgagccaccgcgcccggcccttgtttttgttttttaataataattctgctgtctgctgtgtactagaacccatgcctactgcttggggtataatgtagtaaatgtagtaaaaacaatatccgccgggcgcggtggctcacgcctgtaattccagcactttgggaggccaaggagggcggatcacgaggtcaggagagcgagaccatcctggctaacatggtgaaaccccgtctctactaaaaataccaaaaattagccaggcgtggtgatggacgcctgtagtcccagctactcgggaggctgaggcaggagaacggcgtgaacccgggaggtggagcttgaactgagcggagatcgcgccactgcactccagcctgggcgacagtgcgagactccgtcttaaaacaaacaaataaataaatatgtttaaaacaacaacaacaataaccagccaggcgcggtggttcactcctgtaacccgagcactttgggaggccgaggtggatggatcgcttgaagccaggagaccagcctggccaatatggtgaaaccccgtctctacaaaaaaatacaaaagttagctgggcatggtggcatgtgcctgtaatcccagctactcaggaggctgaggcacaaggctcacttgaacctgggaggcacaggttgcagtgagcatagattgtgtcactgcactgcagcttgggtgacagagcgaggctctatttaaaaaaaaaaaaattaattgaggggccactcccttctagagtggtgagaaatgccgtgcaccgaaagcttcatttgatggtcaaaaccaccctagcaggcaagaaagcatggctcagaaacatatgttcaaggtcaccctgcaagaagtcggtagtaatcggtttcacacccgcatctaacttattctgggtcatctctaccagattagaggggtcctagagggaagcgactgctcagcttcctttccctagggtccccattcagtggaggtctggctctcactgacccattgttagcaagaggaacagggaggtggccaggggtggaggggcagctgtggtcactggcccagtgggagggagctaggccactaggaaccggtcaggccagcaccatccctatccccatgctagccaccacacccaccagctctgccacctccctgctgcatcgaccacttagctctggcagtataggcagcagggcaggctggggcatgctgatacccgcctctgtctgggaagtcgaaggaacagaacctgttcaggctggcggctcatttggatgaacagggagtgtgtgaccttgggcgttgagtcctctccactccctgggcctcagtctccccaacatcaaagaagaaggcaaatcacctttttttttttttttgagatagggtctcgctctgtaacccaggctacaattgtgactcactacagcctcttgacctcccagctcaagtggtcctcccacctcagcctcctgagtagctgagactataggtatagcctcgcaccaccacacccagctaattttttttttttttttttttttttttttttttttgagacggagtcttgctctgtcgcccaggctggagttcagtggcgggatctcggctcactgcaagctccgcctcccgggttcacgccattctcccgcctcagcctcccaagtagctgggactacaggcgcccgccactacgcccggctaatttttgtattttagtagagacggggtttcaccattttagccgggatggtctcgatctcctgacctcatgatccgcccgcctcggcctcccaaagtgctgggattacaggcgtgagccaccgcgcccggccacccagctaattttttaaaaacattttgtacactttgggaggctaaggcgggaggatcacgaggtcaggagctcgagaccatcctggctaacacaggtgaaaccctgtctctactaaaaaatacaaaaaaattagctgggcgtggtggcgggcgcctgtagtcccagctactcgggaggctgaggcaggagaatggtgtgaaccagggaggcggagctttcagtgagccgagatcgcgccactgcactccagcctcggagacagagcgagactccgtcccaaaaaaaaaaaaaaaaaaaatttgtagagacagatcaagtctcactttgttgctcaggctggttttgaactcctgggctcaagcaatcctcccgcctcagcctcccaaagtgctgagattacaggcatgagccaccacacctggccaaatcagctattctgaaaggcccctttaatctctatgagccccagactttcaaactgtaaggaccttaggactgtaactaaagttctacagagcctaaacccctcagctaaagagcctattgttggaaagttctgagtccaagattctatctttggaacattctagaattctccaatttgtctaacccagaattctgagtctttctgtaccacattctacctaacccagggttgcactgctctggaagtctagatggatggtatagtgcagctggtaaaagcatgagtaagaagtcagacttcaaaaattcaaatctgagggccgggcatggtagcttctgcctgtaatccttgcactttgggaggccgaggggggaggatcacttgaggccaggagttcaagaccaacatggccaacacaatgagaccccatttcttaaaaaaaattaaaataaaatcatcaaatctggcagcaccaccgtccaaccctgaccacagtacctcagtctcgtaatccgtaaaatggggatgaaagttcacctcataggactactgtaagaatccacctggtcagaaggtgcaggaagaattcagagctctgagaattgaggcctcaggaagaagagactacaggaataaaaactcgggcatttagaatttcagagatacacaaacaatactttgttaactgttaaaatagataaatgagcaagtctgtgcagccctaatgccagctgtaagtgactctttttttttcttttggtagagatttagtctctctcgcgcctgtggttaggctggtctcgaactcctagcctcatgggatcctccccggctcgatctcccaaagtattgggattacaggcgtgagcacggcgccatgatccccaaatttccaagattctcagattccatactgacattctctggctctcaggaaatgccaaccctgggtgtggggctgtcgcggggacaggcggtggggacgtcggagccaccagggggcggtcacgcccggacccccgccaggagggcggactgcgcctgagctcaggcccggggaatgcgcagcgggcccgggcaggtgctgtacatcccggggcaagggagctgggccgggcggggtacaagggcggggcgcgggggtggcgcgggccgtgtgtctgttcccaggcctctgcccctgacctctgcctccgagtcctctcccatgtgctcccctctagctctagctccgagctctcccgcgggctctgggccagccgcaggtactctcccctgggctcctctctccgctccacccctggctctccttccctggcctcctctgcaccccagccaggttctttagggctaaggatcctgtggacttcctggaggagtcatcttcagtaggaaccgggtcagagagccagactgagctgggaacacccaggctggactcctacagccctgtcgggtcacactgaatctggagaggctccactgtctctgggactcggtttcctcctttgtggacgtctatggaatgggctagggcctttcttgctctaagcctctacttgggcttgttatttagcttctctgtgcctgtttcctcatgtggaccatgggaagaattaataccttcgcctcaaaggggtatgaggattgagtgacataatttataagccgtgattagaacaatgcagtgcgcgaaataaagttcacacatacaggattcataattaccagatgtccttggctgttcattataataacacagggtctggcaacagagtgaggggtccagactcaatgtaatttttttttcccctaaaagggccctttcaactctttctgagatcatacaagccctgagttttgacacccagggtctcaacttcctgagcccttgcctctcagagtcctaaatttcccctgtacattcctgagtctggccagtgatcaccctcagtcacttagggacgggagggctgggagagccctggaagattccagacagaagctggcaaaagcccagggtgtgggcaatatccactctccagcctccgtttctccactcgtaatgaggagtccttccctggggtcagcaaaccttattcaaagggagacctctcagtcacccaagattcctctagacaatgcgagctttcctacctacctacctaccagctctgagcttggtacacccagagccctgttttggcaaccacggttattatttttaatttcatttcaggttatcatcaaatgcccttcaagcccagacattgggaaacactcctctctcatcagatgctcgcctcccccattctgtttttaatcccccttcttaggacgcatgggggttgagagaacggggagatagacagagggaggtgcctggtcctgccctccccccgcctcaaggacagacagacacctccagaattagcctctgtccctccttatctcccacaataccccaggtcagacagatgggcgtggaggtgacatttctcacctcagggtcagggcaaggagccctgaggcagaaggttagtcagaaaatctggcgggggcggatggaatcccgtcccccagagagctgcagaagaaggaggaggcagaatcctgaccctacaaactctactgcctgtgtgagctccaagcctcagtttaccccttcctctccgtgtaatggttaaatgcccggctatgcaaacctcccagaatccaatagccgctttccggaattctgccctgggttctagaactacctctgcaaacccagctgtttcccaccccataaggcaataggggagcccacctccgccagggggtgccctagggcggatgtcccttctctggttaggcaggtctgacgcccaggttaatgacatgttgggttcgctcagcggcacagaggaggttggagatctgcctcggtgttttctctcctaccccgcccccatccccgagccgaaaagtcgggggagagccgggacacagcctccggagggaccccgggtacctgtcctgctccacttcaggaacccaggctccactatccctgccccacccttaattctgctcagagacctagaagatcggtcgagacagcagcttgaggctggcagggtggtcacccattccaccttgagccccaccagtctgagcctctcatttctgaccaagactcggggattcgaacccctatactacccaaagactcggcttcctagagccccccagttcgagggactcaggaattccagctccaacgtctccccgggatgaaggggtagaatccctccattccaagaattcaggcatccgaacccgctttccttccctccagtaaaacaggcaacggagtttccttctaaggatccaggtgtcggcgcgccccaaattccgccctgggacctggcgtccgagtcccctcccaatcctcccagggacgcgggtgttgggctttttcagggcctctggtccccaggagggtgaaactcacggatccgggcagatcctggcacctgggggcttcctccagctcgggctccggcttggggagcggagaacggggcggggcaggagctgggaacaggttagacgacgtgacttgggctggagggaggcgggtcccggtggggagggggagccaaggtcgcctcgagcaccttgggacttgtagtcccggagggacaggacgtagcccaagacgatcccatttggattcacccagagtccatttcacagacaggaagggcgaggcccagaagccgagagcgaccaggccagggagatacagaagagccgagacgcctgcctcgctgtggctggagactgactcctgagcccttgccccaccccttcaggcgcactatcccctttcctgatcagtatcccccagggtctctgagcccgaatctccccgtcgataaaaagcgcgggttggatcttcaaaggatgtcccagcaagagttcaaaatcttagtttggactacaacccccagcagcctccgcgaccgccctcgggcgactctttgcctcgggtcctgtgggaattgtagtcctggagcccgcagggctgcaccccggtgtctctctcgcccacgcgaaggaaaccgtctggagatcctggataggggaaacatttccccttccccttgaccctccctccgctctggaaagcctctcccacctggggagaaggggtgccccaattctggagtaggatcctaaatcttggcagagggggcgggaagtggcgctgacacactggccaggaatgcagtcgggtcaccctgtctagccaccgtctcgcggctccaaccgccgcccaacgcggggcggccccagtgggaagggaagtgggtgcgtcccccaaatctgtgtccacgtgccgctgtttacacgctccctggggcagggaggagtcgccgatcaggtcccttcctgaaagtcatcgaggtttcccacgcatgagactaaacccccgagggcatctacaagtcccatttgatccacaaacgctacaccgtgcccagcaccactccacgcgtgtggggctcctgggtccgaggctccgccctcgagaaccacaagctcctccccctatgtttcccgctcccccggagtccagaagccccgcccctggctggaacttcacgccctccggacggattgcccctatttctccattttcccgcttctcccagtcaagttctgaacttgtgaggcatctgggcctccccagaagacatttaacacagaaagcacagccctactaactagtattcttacctgtctcttcaagaatttcagaccaatcgaccgtcctgtctctttaaggcttaggaagagcagtgtggctgcccctttaaggaggcgttgcaacaaaccatattggacagacgatgggggcgacccatcgggacccgacgggcctctgactccagcaatacagcgaatcagcggctttcgggaatacatttttcggaaaaagacttcttcctcggttttctgctctgcacacgttgaaattttccccagtttttcctgcagatcgggagtcgagcaatgcctacccccgcgctcccgcaccagttgggcgctcccggatgatgccctacccctttggatccacgtggtctgcaacctggtgcgagcagcccgggctacagggttgcctgaggtgtgggtcccaggatggaggagccccaggccggcggtgagggtgcgggttgacggggtgcggagggtgcgttggtggaaggagaaaggggcgtccgagagggttcgggcggaaaaggaggcgtacctgcaagcaggacttgcgaagagcgtgcattcccagtgggcgaacgggaattcgaacggagagagggttatcttgtggggggctacccgtggagagcaaggcgcccccaggggttggatcggtgaaattgaggtcgcccctggggaacaggtgggcagaaaggagaaaccaggttgaggggactggagtgctcacgaggttaagaccaatggaccgataggcgcgccctgcaagattggaccggcaaggaggtgtcagtcgaccccatttccccttctgctgcagatgctgctcggttctcttgtccccccaactttaccgcgaagcccccagcctcagagtcccctcgtttctccttggaggcgctgacgggtccagatacggagctgtggcttattcaggcccctgcagactttgccccagaatggtgagtggtcttgttgacggaaaagagggtcccggtccagaccccaagagcgggttcttgaatttgtcacaggaaagaattagaggtgagtcacagagcacagtgaaagaaacaagtttattggaaactactcctttacagagtagagtgtcctcagaaagcagggggagaaacccacagccctttgttagtatttctacttataagaaactataaggaactatagttaaacttggagtgtgcagataagctcactaaaggtaggggctattggtgttatccacgaccattaatcctgcaacctaagcttgctcatttatgttatatttaagtaatgggggctgcattcttaggacatttggacattctgcaggcttggtggaacatgttctgtatggccataaatattctgtaattataattggtggtcagcctgggatgtggttattttcaggccataagcatgaaccttgtaagtgcctagctactcactttaagatggagtcactctagtcatgttttattaaaaaccagaggccagccaggcgcagtggctggtgcctgtaatcccatcctttgggaggccgaggcgagcagatcacttgaggtcaggagttcaagaccagcctggccaacatagtgaaattgtctctactaaaaatacaaaaattggctgggcgtggtggcaggtgcctgtaatcccagctacttgagaggctgaggcaggagaatcgcttgaacccaggaggtggacattgcagtgagccgagatcatgccactgcactccagcctaggcaacagagcaagactctctcaaaaaaaaacaaaaaaaaaatcaaaaaaccttccctctcctgttccacttaagcctctgccctccctgtttctctctgtagcttcaatgggcggcatgtgcctctctctggctcccagatcgtcaagggcaaattggcaggcaagcggcaccgctatcgagtcctcagcagctgtccccaagctggagaagcgaccctgctggccccctcaacggaggcaggaggtggactcacctgtgcctcagccccccagggcaccctaaggatccttgagggtccccagcaatccctgtcagggagccctctgcagcccatcccagcaagtcccccaccacagatccctcctggcctgaggcctcggttctgtgcctttgggggcaacccaccagtcacagggcctaggtcagccttggcccccaacctgctcacctcagggaagaagaaaaaggagatgcaggtgacagaggccccagtcactcaggaggcagtgaatgggcacggggccctggaggtggacatggctttggggtcgccagaaatggatgtgcggaagaagaagaagaaaaaaaatcagcagctgaaagaaccagaggcagcagggcctgtggggacagagcccacagtggagacactggagcctctgggagtgctgttcccgtccaccaccaagaagaggaagaagcccaaagggaaagaaaccttcgagccagaagacaagacagtgaagcaggaacagattaacactgagcctctagaagacacagtcctgtccccgaccaaaaagagaaagaggcaaaaggggacggaagggatggagccagaggagggggtgacagttgagtctcagccacaggtgaaggtggagccactggaggaagccatccctctgccccctacgaagaagaggaaaaaagaaaagggacagatggcaatgatggagccagggacggaggcgatggagccagtggagccggagatgaagcctctggagtccccaggggggaccatggcgcctcaacagccagaaggagcgaagcctcaggcccaggcagctctggcagctcccaaaaagaagacgaagaaagaaaaacagcaagatgccacagtggagccagagacagaggtggtggggcctgagctgccggatgaccttgagcctcaggcagctcccacatccaccaagaagaagaagaagaagaaagagagaggtcacacagtgactgagccaattcagccactagagcctgaactgccaggggagggacagcctgaagccagggcaactccgggatccaccaagaagaggaagaagcagagtcaggaaagccggatgccagagacagtgccccaagaggagatgccagggccgccactgaattcagagtctggggaggaggctcccacaggccgggacaagaagcggaagcagcagcagcagcagcctgtgtagtctgcccccgggaaactgaggaactaaagaaagctgaaggtgcccacctgggccaccagaaggtgacacccccagaatccctccccagagactgcaccagcgcagcc

SEQ ID NO: 2 - DNA, 38166 bp, Human - part of chromosome 19
ggcgccggccggactgtgcagcggggtcgacccgcctccctcatgaatattcagcgagaggccgggtcgtggacatcctcgagggctcgctccaccttattacgagaccattggctaacctgcccgtcaatccgctagggcagagcaatcgggatactgcgcgtgcgcacggaaaagcgagggcggctgactctcgggtgaggcggtgcgggaggcgtcactgaggatcgtcgagggccaatcaaaagaaaacatggaagggaaagagccgagagactcgatctcattcactagaatttggtcctcctgcgcctgccaagattgtctgagtattgatcgaacccaggagttcgagatcagcttgagcaagatagcgagaacccccgcccctccacctcgtctcaaaaaaaaaaaaaaatcgtctcagtagcgaatagtctaacggagaatgacagggaaattggtgatcctttctgggcccaagagttagaaatggctttgcaggccgggcgcggtggctcaagcctgtaatcccagcactttgggaggctgaggcaggtggatcacctgaggtcgggagttcaagaccagcctgaccaacatggagaaaacctgtctctactaaagatacaaaattagccgggcgtgctggcaaatgcttgtaatcccagctactcgggaggctgaagcaggagaattgcttgaacctgggaggcagaggttgcagtgagcagagatggcgccgtcgcactctagcctgggcaacaaaagcgaaactccatttcaaatattaataataataactaataaataaaacataaatgctagcttttgtttgtttcttcaacaaatagctatgtggcatctaccatgtgtctgatcctgtgctggcccctgggaacagaaaggtgaccatgacagcctcagcacctgccctcaaagaacagatttttttccttgagacagggtctttctctgtcgccaaggctggagtgcagtggcacagtcacagctcactgcagcctccacctcttgggctcaagcgatcctcccacctcagcttccagagtagctgggaccacaggtgtgcaccaccaagcccagctaagttttattttttaaatttttttagagacgaggtctcaccacgttgcccaggctggttaaactcgcaggttcaagtgatcctctcccctcagcctttcaaattgttgggattacaggggtgaggcaccaggcctggcctcaaagaacagatattaaatatacaaatgaatatatgattacagcctggagtggtggctcgtgcctgtggttccaacactttggaaggccaaggcgagtacattgcttgagctcaggagctagagaccagcctgggcaacatggtgaaaacccgtctctacaaaaaatgcaaaaattagctgggcgtggtggcgtgcacctgtagtcccagatactcaggaggctgaggtgggagaatcacctgggcctgggaggcagaggttgcaatgggcagtgattgtgccactgcactccagcctgggcaacaggagtgaaaacctatctcaaatgtgtgtgtgtgtgtgtgtgtgtgtgtgtgtgcgcacgtgtataatcacaagtacaaaagtgctgtgaaggaaaacttcaagtcaccataaagattgattatgggctgggtgcagtggctcatgcctgtaatcccagcactttgggaggccaaggcagatggatcacgaggtcaggagttcaagaccagcctggtcaacatggtgaaaccctatctctactaaaaaaaaaaaaaaaaaaaaaaaagccaggcatagtggcatgcatctgtaatcccatctactcgggaggctaaagcaggagaattgcttgaacccaggaggcagaagtgagccaagatcacgccactgcactccagcctgcgtgacagagcaagactccgtcccagaaaaagaaaaaaaaaaaagacttattatgacaggatgtctactgtcaactgtggggtgtgagtgttggccaagtgatcagagaaggcttcgtggaagaagcgaggtttgagtagagccagaaaataattagaagagatcaaccagcaagaggggatggatgagagaagtgagaaaggtgttccagggagagagaccatcatacacaaaagctctaggccagaagaaagctgaggcctgtgagtgctgaaaggaagcctgtgggggtggagctctgagttgagcacagggagcagagaaagggcagctggaggggaaggcaggggcagatcgaaatctcttttttaaattaattaattcttaatttatttatttttgagacaaggtctcactctttcgcccagactggagtacagtggcacaatctcagcgcaccgcaacctctgccacccaggctcaagcaattctctggcctcagcctccctagtagctgggattacaggtgcgcaccactactgcccagctaatttttatacttttagtagaaacggggtttcactatgttggccaggctggcctcaaactcctgacctcaaaagatccacccacttcagcctcccaaagtgctgggattacaggtgtgagccacccttcccggctgtatttttggagacagagtcttgctctgtcccagcctggagtatggtggtgtgaatttggctcattgccaccttgacctccagggctcaagtgatcctcccacctcagcctcctgagtagctgggactgcgggtacacgacaccacgcctggttaattttttttaattttttgtagagacgagggtatctcactatgttgtccaggctggttgaactcctgagctcaagcaattctcccacctcagcctcccaaagtggtgggattacagacgtgagccactgtgcccggcttaatttatttacataaatttttttatgtttacttttctatctcctacaggaagaaaatatattttgttattgacagggtctcgctatgttgcccaggctggtattgggctcaagccatcctgttccctcagcctcccaaagtactgggattacaagcgtgagcctctgcatccagcccagatccaaaatctttactgtcacctacagagtcctctgtaactagcttactgctcatcatccccataccaacccaccttactgctctgatctcctcctctctctcccccagctcattttgtttcagctatgctggtctccttgctgtctctaaaacataacaagcacatcccatctcagggcctttgcaccagctattttgtctgcctggaatgctgtttcccctgatagccatgtggctgacacactcacctccctcagctctttgctcaattgtcaacttctcggcccggcatggtggctcacacctgtaatcctaccactttgggaggctgaggtgggcagatcacctgagatcaggagttcgagaccagcctggccaagatggtgaaatcccgtctctactaaaaatacaaaaattggcaaagcatggtagcacataccagtaatcctagctacccgggaggctgaggcaggagaattgctggaacccgggaggcagaggctgcagtgagccaagatcatgccactgtactccagcctgggtgacaaagcaagactctgtctcaaaaaaaaaaaagtctccttctcaatgagggcttcctgaccaccaaattaaatctacctcctagacacacacacacacgcacgcacgcacgcacacacacacacgcacgcacgcacacacacacacacacacacactatatcccctttccctgctttattgttcttgagagctcatttaaccatgtgacatgctgaatattttacttatttattttgtttagaaagctcctggctgggcgcgggggctcacgcctgtaatcccagcactttgggaggctggaacaggtggatcatgtgaggtcaggagttccagaccagcctgaccaacacggtgaaacctcatctctattaaaaatgcaaaaattagctgggtgtggtgtcgcatgcctgtaatcccaactactcagaaggctgaagcaggagaatcgcttgaacctgggaggcagaggttaacgctgagccgagatcgcgccattgcactccagcctgggcaacaagagtgaaactctgtctcgaaaaaaacaaaagtcagctccatggcaggagtgatggctcacgcctataatcccagcactttgtgaggccgaggcgggcggatcacttgaggtcaggagttggagaccagcctggccaacatggtgaaacctcatctctactaaaaatacaaaaattagccgggcgtggtgacacatgtctgtagtcccagctacttgggaggctgaggctggagaatggcttgaacctgggaggtagaggttgcagtaagccaagatcgcgccattgctctccatcctgggcaacagactccgtctcagaaaggaagaaagaaggaaagagagaaagagagaaagagacagagagagagagagaaagggagaaagagagaaaggatggaaggaccctgacaagcactgttgcataaaagtttcttttctctctcttttttttttttttttttttttgagacagggtctcacttctgttgctccagctgaagtgcagtggtgagaacatggctcagtgcagcctcaacttcccaggcttaagtgatcctgccacctcagcctcctgagtagctgggactgtaggtgtgcaccaccgtgcctagctaattttttgtatttttagtagagacatggttccgccacgttgcccaggctggtcttgaactcctgggcttaagggatctgcccgccatggcctcccaaagtgctgggattaccagcgtgagccactgtacccagcctgagtataggtttctgataaattttaggatcatattgtttggactgggtaagaatttccagaactctaatgaagaaactgactggtttatattttattttattttattttattatttttgagatggattttcactcttgttgcccaagctggattgcagtggcacgatcttggctcaccacaacctccgcctcccggtttcaagtgattctcctgcctcagcctccccaggagctgggattacaggcacccaccaccatgctcggctatttttttttttatttttttatttttagtagagacggggtttcaccatgttggccaggctggtctcgaactcctgacctcaggtgatccacctgccttggcctcccaaagcgctgggattacaggcatgagccactgtgcaaggcctaggctggtttataaaattgctaaaccaagcagaacatgaattaaataccaaggaaatactctcctagattgtcatgttacatcagccaatactaaaattgtcaagatacacaatttgaatgaactccatggtccaagtcgaattatctatgatattacccatctaataaacagcactatgtcccttaatgggagaaaaagttggagaatttaagagaatatcaatccaatgttggttgggtgcagtgaatcatgtctatattcccagcactttgggaggccaaggcaggaggatcacttgagcccaggaattcaaggccagcctcggcaacacggtgagatcctgtctctacggaaaattaaaaaaaaaaaaagagagagattagtgggatgtggtgcctatagtcccagctacttgggaggctgaggcgggaggatcatttaagcctgggacgttgaggttgcagtgaaccatgagtgagactcatctcaaaaaaaaaaaaaaaatggcgatcactagaggaaaaaaaaactaaagtggggtttgcgggtagtgggagggcccttcctgctaggttgcactatgatctccagggaggctccacgggagaatcatttccttgtctttttcagtttctagagccaaattctttgcataccttgcattccttggctcggaaccccttccctaaccttcaaagctggcagctagcctctggctcaagtgtcacatggcctgtctctgtcttcctatccaatcttcctcttataagaacattggagccaggcatggtggctgacgcctgtaatcccagcactttgggagaccgaggcaggcggatcacaaggtcaggagttcgagaccagcctggccaacacagtgaaaccccgtctctactaaaaaaatacaaaaaagtagccgggcatggtggcaggtgcctgtaatcccagctacttgagaggctgaggcaggagaatcgcttgaacctgggaggcagagcttgcagtgagccgagatagtgccaatgcagtccggcctgggcgaaacagcgagactccgtcgcaaaaaaaaaaaaataataataaataataaataaaaataaaaataaaataaaaaaataaaaataataaaataaataaaaattattttgagacaaagtctattctgtggcagaggctggaatgcagtggcgtgatcacagcttactgcagcttctacctcctgagctcaagcgatccttccaccttggcttcctgagtagctgggacctcaggtgtacattaccacgctcagctaattatttatttatttattatatttttgtgacggagtttcgctcttgttgcccgggctggagtgcaatggtgctatctcagctcactgcaacctctgcctcctggattccagtgattctcctgtctcagcttcctgagtagctgggattacaggtacatgccatcacgcccagctaatttttgtatttttagtagagacggggtttcatcatattggtcaggctggtctcgaactcctgacctcaggtgatccacctgccttggcctcccaaagtgctgggattacaggcgtgaggcaccacgcccggcaattttttttttcttttttttttttcagacagagtcttgctctgtcacccaggctggagtgcagtagcgtgatctcggtttactgcaacctccatctcccgggttcaagcgattctcctttctcagcctcccaagtagctgggactacaggtgcacaccaccacggcgggctaatttttgtatttttagtagacaccaggtttcaccatattggtcagactggtctcaaactcctgacctcaggtgatccatctgcctcagcctcccaaattgctgggattacaagcgtgagccacacacctggcttaatttttttatttttgatcgacacagggtctccctatgttgtccaagctggcagagatttttgtttgtttgtttgagagggaattttgctcttgtagcccaggctggagtacaatggtgcaatcttggctcaccacaacttccgcctcccgggtttaacagattctcctgcctcagcctcccaagtagctggaactacaggcacctaccaccacaccaggctaatttttgtgctttttagtagagatgaggtttcaccatgttggccaggctggtcttaaactcctggcctccagtgatccacccgccttgacctcccaaagtgctgaaattacaggcgtgagcaccgcgcctggcctctcaacctacaatttcaacacccaaggaaacagcccaccatgagtgagaaccagcagacacaacaaactataggattagctgcctccaaacttcaggtgatagattatcaggcatgtacttgaaactaaaggacacaaaagaagaatccgaaatataaaataaaggattggacttgtgtgaaaagaatcccttagaaagggctactttcaggctggccatggtggctaatggcctgtaatcccagcactttggaaggccgaggtgtgtggatcacctgaggtcaagagttcaagaccagcctggccaacatggtgaaaccccgtctctactgaaaatacaaaaattagccaggtggggtggcagatgcctgtaatcccagctactcgggaggctgaggcaggagaatcgcttgaactcaggaggcagaggttgcagtgagctgagattgcgctatcgtgccccagcctgggcactagagtgagatcaaaaaaaaaaaaaaaaaaagaagaagaagaagaaagggctactttcagactgccttgccaaaaatcataaccacaatgatgagcatgtattgagtcaaaacagaatcaaaagagaagaaagtcaatttctgtgcaaactacttttatttataaggaaagtttctctattttgtttataaacattaaaccagtgctgtgtgaaggcacttaattggggagaggtggggcagggatcctggtagagaccaatgtttcccacccagaccccaagactgctgggagagatggtgtcagcagtgactcccaggaatatccagtggtgtggtggcccatcccaggcccggctgggcaggtggctggcttgctgggggatgtgatgatggtggtaggcatgggaggcactttggacgggatctgatttggcaaaaggaagtggtttcctgtccccagtgatttccagcccttcccagacctcccaaggctaaggcagattactaaatttaaggctggggccctccttcttccctggacttccaggagaacagagaaccggtggcaaggaccaccaccagcagggtgaggggtgcagataaaggcagcaaaaaacagagggagaggtctggagggaaggcaggaatgcttgtttctgtcagcctcagaaacctccttctatcctgctagactttactcctttgaggcttcaccctggggaacagctggggagagacaggatcttcagacatcaggagctcccacctcctcatcccacatgcaaatccgctgcctgtctctatcctcccaccccttcctaaggggacctctcagcacctcccaaactgctccagaatccaagttctgtgtcacctccaagaaccagatggaaccttccaatcagagcctccactgatgaaatggaatatttccagtgtctcctaactgccataaggagaagcccacctctctctaacaccttggttgtctttttgggtcccacctccatatttaaaaaatctcctctctcagggccgggagcagtgggtcacacctataatcccagcagtttgggaggccgaggtgggtggatgacctgagctcaggagttcaagacaagcctggtcaacatgacgagaccctgtctctactaaaaacacaaaaaattagctgggcgtggtggtgcatgcccgtaatcccagctacttgggaggctgaggcaggagaatcacttgaatccgggaggtggaggctgcagtgagccaagatcgcgccactgcactccagcctgggcgacgcagctgaagctgtgtctccaaaaacaaaacacacacacacacacacacagaaaaaaaaaaccaaaataaaaaaatctcccttctcaggaatgtaacggaatcttccttgccttctcccctaaccctaatagagaattttcctcagttacactgtaattttattaatggatttttcctcattctgcccaatgcagtgtaatgaaagcttcctctccatctgttatattatatataaatatatattatatatttatatattatatatttatatataacatataattttattgtcacccaggctggagtgcagtggcaccatcagggctcactgcaggatcaatctcccaggcttaagcgattctcctgtgtcagcctcctgatgagctgggattacaggcacccgccaccacacccggctaactttttttttttgtatttttagtagagatggagtttcaccatgttggccaggctggtctagaactcctgacctcaggagatccgcccgccttggcctcccaaagtgctgggattacaggtgtgagccacctggccgggccctccacttccttcttgtacattgctgaatccctgtgtcagccctagaggtccagtcttttgccctctcccagccttaatctacaattctgtaacccacccaccatcattaaaatgagattcttctttgtcgcttcccttggctaaaatggattattctttaacctctccaccaatacaaccagggatgataataaaaacattggattgagcagaaaccaatcaaataactagtaaggcagtactggcgagcaccctacatcctgacagctttataaagggcgcttccagccaggtgcggtggcacatgcctgtaatcccaggactttgggaggctgaggcgggcaggtcacctgaggtcaggagttcaagaccagcctggccaacgtgatgaaaccctgtctacacaaaatacaaaaaaaaaaaaaaaattagccgtgcgtggtggcatgcgcctgtcatcccagctactctggaggccaaggagggaggatcacttgagcccgggaggcagaggttgcagtgagcccacatcttatcactgcactccagtctgggtgacaaagcaagactccatctcaaataaataaatacaaattggccgggtgcggtggctcatgcctgtaatcccagcactttgggagaccaaggcaggtggatcatttgaggtcagtagatcaaaaccagcctggccaacatggtgaaaccccgtctctactaaaaatacaaaaagtagccgggcgtggtggtggtgggcgcctgtaatcccaggcaggagaactggttgagcccgggtggggggggcccgaggttgcagtgagcacagatggcgccattgcactccagcctgggcgacagagcgagactccgtttcagaaataaataaataaaataaaaataaaaataaaaaaataatagaaatttaaaaataaaataaagggcttttcctcacctactccactaactataagggacccttacccccgacattactattaaatataacggacttttcgtctcctccccatgagcaataatgagcttttcagacctccctctcccaatataacggtttgttcctgttgcctcttctttttcctgtgggatcccccttttccccaacccccaactgtcgggaggtccccatgacttctcccctgggctcaccccgaagtagttccgcggcacgtagccctcctggccgtgcagcgcggcccaccaccagtcggtctcctccggcccgtccctccgcagcacggtgaccgactcgccctcgcggaaggacagctcgtccccgaactcggcgctgtagtcccagagagcgtacactgccccgctgttcatcagccccatactctgctcgacgtctgaaacatgccacggaggggaaggtgagagcctggcccagggggtccaggaacaggggccacgtggggtccaggacagaccctggaatttggcgcctgtcccagcaaccacctgaaatgttgtgtgtgcccatggctgtggatgggaaccggagctggagtcagatgccgggactggccgtctttgagcgttcgaggaaactgggggaggcatgccagtgggccacccactcccgaggcagggtcagaggctcccatttcttttctttcttttttttttttttttgagacagagtctcgctctgtcgcccaggctggagtgcagtggcacgatctcggctcactgcaacctccgcctcccgggttcacaccattctcctgcctcagcctcccgagtagctgggactacaggcgcccgccaccacgcctggctaatttttggtatttttagtagagtcagggtttcaccgtgttagccaggatggtctcgatctcctgaccttgtgatccgcccacattggcctcccaaagtgctgggattacaggcgtgagccaccgcgcccggcctttttttttttttttttttttttgagatggaatttcgctcttgtcgcccaggcaggagtgcaatggtgcggtctcactgcaacctccgcctccggagttcgagccattctcctgcctcagccttccaagtagctgggattacaggtgtgcgccaccatgcctggccaatttttgtatctttagtagagacggggtttcaccatgttggtcaggctggtatcaaactcctgacctcaagtgatccacccgcctcggcctcccaaagtgctgggattacaggcgtgagccacctggcccggccctcatttccttcttgtacattgctgaatgcccgtgtcaaccctagaggtccagtcttttgccctaccctggcgcttagcttaagtggtacagtctctaaggaagattcgcaccttccttgaatgatagggtcctttaagttggctcatctgcctctttcttttcttttcttttcttttctttttggagacggagtcttgctctgtcgcccaggctggagtgcagtggcgcgatttcggctcactgcaacctccgcctcctgggttccagcaattctcctgcctcagcctccaaagtagctgggactacaggcccacgccgctacacccggctaaattgttttatatttttaatagagacggggtttcaccgtgttgcccaggctggtttggaaatcctgagctcatgcaatccgcccgcctcgagcctcccaaagtgctaggattacaggcatgagccaccgcgcctggctttctttttcttttcttttctttttttttttcagacaaggtctcactctgccacccaggctgcgggagtgcagtggtgagatcaagcttactgcagcctcgaacttccagattcaagcaatcctcctgcctcagcctcctcctgattctttatgttattattaaatattttgtaggccgggcacagtggctcacacctataatcacagcactttgggaggccaaggcaggcggatcctctgaggtcaggggtttgagaccagcctggccaacatggcaaaaccccgtctctactaaaaatacaaaaaaaaaaaaaaaaaaagttagcgggccgtggggcccttgcctgtaatcccagttactcgggagcctgaggcaggagaatcgctttcaccgaggaggcagaggttgtagtgggctatggtgccattgcactccagcctgggtgacagagcaagactctgtctcaaaaaataaataaataaaaataaataaatatttcgtagaggtcaggtgtggtggctcacacctgaatcttagcactttgggaggccaaggtgggcagattgcctgagctcaagagttcgggaccagcctgggcaacactgcaaaaccccttctgtactaaaaatacaaaaaaatgagtcgggcatggtggtgagcacctgtagtcccagctactcaagaggctgaggcagagaattgcttgaatccaggaggtggaggttgcagtgagccgagattgagccactgcactccagcctgggtgacagtgagactctgtctcaaaaataataataaataaatatttgtagagacagggggtctctacaatgtcttgtagcctgaccaggctcacctttcaaatatataaccctctgtctcacccataagtcctaggacctgcctcactccaactctccgtgaagttccttgcccacaccgagatacaactggctcctccaggtgtgaaatgaccctgtgcacaatccccgtggcacagcctacttcgccctgcccgtcggggaaccaggtgatgtagcctgccccctggagagatagggtacagccttgtgtcttcctacaagcccctttctggcagctgtagcctgctcacctgccagtggtgtggcaatgcctctcccacaagtggcagagcccacctgcccagagccctatgccaggtagatggcagggttgaaacgttcagctcctcacccttgaagatgtgaaaggtgagcagaccaatcttcacagccactctcctccccaaaggtgtccagctcgcatagcacagcctccatgtccccttttcccttaggagggcatagtccccccacccccgcaagcggtccatccctcatcctcctcctcggcaatcctgccaagtggttggtacagcccccatacccttctctccctagtagggggtagttgctcccctccccgctcctgcgcacccgccaggtacccaggcgccagcagccctgcctcgcacctgccaggtaggtggcgcagtcagcataaccctcgcggtaagggtcgcacttctcgaaggcggtggcgccgtcgctgagcgtggtggcgaagattgcagcgccgtgctgcaccagcgccatgcagatgactgtgtcgttgcacgacgccgcgcagtgcaagggtgtcctaggcgtgggggtggggggttgcggggaacgatgcgtgagaggctgcgcgtccgcccacgggggacccagcccaccgcgcgggtcggggctcaccagccgtggctgtcgggggagttgacattggcacccgcggtgatgaggaaatccacgatagagtagttggcgccgcagatggcgttgtgcaaggcagtgatgccctcctcgttgggctggctcgggtcgttcatctgagtgcaccgggggagggggaagactcagtcccgcggctggcatctgcgatgcccccgccgtgcccacctcccgctcagcagcgctcacctccttcaccgcctgctgcaccacctccagctccccggtcagcgccgcgtccaggaggagcaccagagggttgaggcgcgcgcggcgggccttgcgcggggagcccgccttccgcagcacagagcgcatctcctgggggacagggcgcagaggtcagcgacttggagggattgttagtatatccatgatctagagtaggaaacagaggtccagggacttgtggcacccatctagacaggggtagaactgggattccctcgggatggggtgagggggtgccttcgatctcctcctagagcctccagttccctgccatagacagggaatcctgtgatttgagaatcttgggccctgaaacttgggagaaagctggggggccatgggattggtggcaaagtaattctatcagttcaaaacaatgattgtggaagccagttatgcaattcacacacagtctcacatttcttttgttaataatgaatgcaatgagacacacatgacaaaatgttaccaggagtgttcattccggatgtttggaatttgagcattttattattccttgtattttccttttctttttctctttttttttttttttttgagatggagtctcgctctgtcacccaggctggagtgcagtgcagtggtgtgatctcagctcactgcaccctccatcccccaggttcaagcaattctcctgcctcagcctcctgagtagctaggattacaggcatgcgccactatgcctggctaattttcatatttttagtagagacagggttttgtcatgttgtccaggctggtctcgaactcctgacctcaggtgatccacccacctcagcctcccaaagtgctaggattacaggtgtgagccactgtgcccagcctcatgggctttcttatttttaattttcctcctgtaagattcatttattctgggctgggcgaggtggctcatgtctgtaatcctagcactttgggaggctgaggtgggaggatcacttgagcccaggagttcgagaacagcttgggcaatatagtgagacccagtctctacaaaaaataaaaaattagcctgacatggtggcgcacacccgtcgtcccagctacttgggaggctgaggcaggaggattacttgaatggaagagaaggaggcttcagtgagccatgatcatgccactgcactctagcctgggcaacagagtgagacccagtctcaaaagaaaaaaaaatgcatttatttattccaagtgtgtgagtgcatagcatttgtgattctggtctttgctgtttccagagtttcagtgattttaagattctggaattcagagatcccaacagccactgaattcaaaattcccagatgctcagttatttcaagtttccaatatgttgtgattgcagaaatgctaggctgtgctatttcaaattgctgaggggccaggactttggaatccaaagattctatgatggagaactttaatatttttctgttagaatttcttttttttgttggtttttttgagacagagtctcgctctgtcgcccaggctggagtgcagtggtgcgatctcagctcactgcaagctccgcctcccgggttcaggccattctcctgcctcagcctgccaagtagctgggactacgggcgcccgccaccacgcctggctattttgtatttttagtaaagatggggtttcaccgtgttagccaggaaggtcttgttctcctgacctcgtgatccgcccacctcggcctcccaaagtgctgggattacaggtgtgagccatcatgcctgacctagaatttcattttaaaagactagaaggaaatggctgggtgcggtggctcatgtgtgtaatctcagcactttgggaggctgaggagagtggatcacctgaggtcaggcaggagttcaagaccagcctggccaacgtggtgaaaccctgtctctactaaaaatacaaaaattaggtggccgtggtggtgcacgcctgtaatcccagctactcaggaggccgtggcatgagaatcacttgaacccaggaggcacagttatagtgagctgagatggcaccatcgcactccagcctgggtgacagagtgagactccatctcaaaaaaggaaaaaaaaaagaaagactagaaggaaatattcaaaatgttaatgatggttccctgtgagtggtgtgattttgtcctctttcttctatttttatttattttccccaagctctctatggtgttggtgtatttctctatagtggaatgtgtaaatttaaagtataaatctcagctgggcacagtggctcatgcctggtttgagaccagcctggacaacataatgagaactgtctctactgaaaatgttaaatattatctgggagtggtggtgcatgcctgtagtcccagccataggggaggctgaggcatgaggatcaattgagcccagtaggtggaggctgcagtgagccatgatcttgccactgcactccagcctgggcaacagagtgagactctgtctcgataataataaccctctattacaacatatcagtgcatgaatttgtgattttataattcaaaatatgagcatctttaattgtcagatttggtgacttcaagaatcagtaataatcagtctatgatactaactttataattattttttttaagagaagagtttccttttattttattttatttgagacagagtttctctctgttgcccaggctggagtgcagtggcgcaatctcggctcactgcagcctctgtctcctaggttcaagcaattctcctgcctgagcctcccgagtagctgggattacaggcatgcaccaccaggcccagctaatttttgtatttttagcagagacggggtttcaccatgttggcgaggctagtcttgaactcctgacctcaagtgatccacccgcctcggcctcccaaggtgctgggattacaggcatgagccaccgtgcccagcctaactttataattctaagatcgtgttcaaacctttaaatgctctagggctctaaaatgttactatcctaagacggtgacactagcgtttgattcttacattctatgattttttaagtttctctgtggccaggactctgtgattctacaatgggatgctcagccatttcaacatgttgttattcatcccctcttgatttcaaaatcctgagcctcaaggttccttgcctttactttcaggagggcctaggaataggcattttgggggggtccacctgacccctgcttctctgagaagtgatctcttcccgctgtctacgcacacggagtgttcaggactgttccatgtggctacaaccctcttcccagtcaagatgcagggaccaagatcagcaggagaccatcccctggtccaatggtgacaacagtaagagcagttaacagttatgtgccaggtattatgctaagcactacattaatgtatttaatcttggcggggtgtggtggctcacacctgtaatcccagcactttgggaggccagggcgggcagatcacttgaggtcaggagttcaagaccagcctagccaacacagtgaaaccccatctctactaaaaatacaaaaattagccaagcgtggtggcatatgcctgtaatcccagccacttgggagactgacgcaggagaatcactttaacccaggaggtggagtccagcacccagccgagactcacttgtttttatttatttatttatttatttttatttttattttttttgagacggaatcttgctctgtcacccaggctggagtgcagtggcgcgatctcagctcaccacaagctccgcctcccgggctcacgccattctcctctcagcctccagagtagctgggactacaggcgcccgccaccacccccagctaatttttgtatttttagtagagacggggtttcaccgtgttagccaggatggtcttatctcctgacttcgtgatccgcccgcctcggcctcccaaaatgctgggattacaggcatgaaccaccacgcccggcctatttatttatttatttagagatggagtcttgctctgtcgcccaggctggagtgcagtggtgcagtcttggctcactgcaacctccgccttccgggtttaagcgattctcttgcctcagcctcctgagtagctgggattggaatgagaccaccacttctcctgttgtccttcccagcttctcccccacctccccttttccctagtttataagacaggaaaaaaagggagaaagcaaaacgctggaaaaaaacagaagtacgataaatagctagatgaccttggcgccaccatctggtcctggtggttaaaataataataataatattaatccctgaccaaaactactggtgttatctgtaaattccagacattgtatgagaaagcactgtaaaacgttttgttctgttagctgatgtctgtagcccccagtcacgttcctcacgcttacttgatctatcgtggccctttcacgtggaccccttagcgttgtaagcccttaaaagtgctaggaatttctttttcggggagctcggctcttaagacgctgatgctcccggccgaataaaaacctcttccttctttaatccggtgtctgaggagttttgtctgtggctcgtcctgctacagaattacaggcacgcgccaccgctccgggctaatttttgtatttttttagtagacagggggtttcaccatgttggtcaggctggacttgaacctctgacctcatgatccacccacctcggcctcccaaagtgctgggattacaggcgtgagccaccgcgcccggccgagactcactattttataagaggagagagcaaagccaggaacagtggctcatgcctctaactgcagcaatttgggaggctgaggcaggtggatcatttgaagtcaggagtttgagaccagcctggccagcatggtgaaacctcatctctactaaaaatacaaaaattagccaggagtggtggcatacacttataatcccagctacttgggaagctaaagcgggaggatggcttgaacctgggaggcggaggttgcagtgagccgaggtcaagccactgcactccagcctgagtgatggagcaagactctgcctggaaaaaaaaaaaaaatagaggagagagcagagcagacacaagagacacagagacagagagggagagaagagagggtgactgctttgattcaggcaagacttctcagtcccagaatgaacccactgttgtgccaagactcagtcatgtccaggtgtatgactcgagattgctgaaggaatgcccggggcagggcacaggcacaggttattggagagaaggagcagagaacatctctatgtggccaagactcccagatggccctccatatagtcacacacagctatcctaaagactacatttcccagcatcccattgcaatgaggctcctggccagtgggagcaggcagagtgatgtatggaactcccaggttctgcctgaaacaggaaagggcactttctcttcttctttctctcttcctggctggagggcagacttggtgacagccatctaggaccatgaaggcaggcttactccccgatggatggcagagccccaggtagatagagcctgggtcctgactccagtgaggtgcctacagtcctgggctgcaaactcttggacttctactcaaaagaggagaaaacttcgatctcatctaagccactatatttggggggctctttgctacagctcctggattcatgtagcaaacataccccggtttcctcctgtattacttaccatgctctgcggctgctctggtgggctgctctgggacggggccgggggtggaatgggagctggtggggcaggagcagggggccctgccctggcctcagatccctcagtgatgggggacagctctggctccggccccccgggccctggccccccatgacgatggaagaggcggctgatgatctgctggtactgtttcttgtgggtagggggcagggccacagcaggggcctgctccatggagcccctgcgtttgaggggccggggaatttccgccaacacccgtgccacctcctccagctcgggcaccgactgtgcctccggtggcagtgctggctgcagcctcgtggggctgagaggccttgctacagggccttcatccacatcgccagcctccagcactggtgtcagcagcccctctatctccggctcaggctccagctcggtggggggtttggggggtcctagccggaacaagagcccatcagaggacaggtccccaggagacacccaacactccctctccacaacttccagggcatacaaccagcacatgattttctgtgtgacctcagggaagttccttgccctctctgggctacactttccttgggctgtgaataatatacaattatgatgcctcccatttattgagcagttagtatgtgcctggcgctttacatgcctaccttattgtaatctcaccactgctttgtgaggtagatacactgccatctccacattaccgaaagggaatctgggcctcagagaggacaagtcagttgcccaaagccatgcagttgggacttgaactcagttctggctgactctagaatctacttctaccaaccgtgatagatgtgattttctgagatcctgagagtttcctctcctaacatctcaggcagaaaactccagcaggaagtagaatcctggtgtttaatgatttcttctctgtcttactcattctgacagtaaagcaggtggaaataaaaatatgcattattggctgagtcgagtggctcacacctgtaatcccagaactttgggaggccgaggcaggcagatctcttgagatcaggagtttgagaccagcctggccaacatggtaaaaccctgtctctactaaaaatacaaaaaaaaaaaaaaaaaaaaaaaaattagctgggcgtggtggcacatgcctgtaatcccagctactcggaaggctgaggcacaggaatcgcttgaacccaggaggcggaggttgcagtgagccgagattgcaccactgcaccactgcactccagcctgggcaaaagagtgagatttcatctcaaaatatatatatatacacacacacacacaaacacacacacacattatatatatagtgtatatatatttttatatagtatgcatatacatataaataatacacacacacacacacggctgagcatggtggctcatgcctgtaatcccagcactttgggaggctgaggtgggtggatcacctgaggtcaggggttcgagaccagcctggccaacatggcaaaacctcatctctactaaaaacacaaaaaattagttgggtgtggtggtgcatgcctgtaaccccagctacttgggaagctgaggtaggagaatcgcttgaacctgggaggtgtaggatgcagtgagctgaaacctcaccactgcattccagcctgggcaagaagagtgaaactccatcttggctgggcacggtggttcacgcctgtaatcccagcactttgggaggccgaggtgggcagatcatgaggtcaggagatcgagaccatcctggctaacatgatgaaaccccgtctctactaaaaatacaaaaattagctgggggtggtggtgggcgcctgtagtcccagccactcgggaggctgaggcaggagaatggcgtgaacccgggaggcggagcttgcagtgagcaagcaccactgcactccaacctggaagaaagagcgagactctgtctcaaaaaaaaagagtgaaactctgtctcaaaaataaataaataaataaaccccaaaacacacacacatacacattatttcattgaatccccgtcacaattctatagggtagatattattaatctctcttcacagacgggaaacagagtttcggacaagtaatttatcttcagtcacacagcaagttagcagtgaagagagactccagcccatctgcttaactcactgatctcacacctcaaaatattaataaattattataactaatatggtagctatttatttgagactgggtctcactctgtcacccaggctggagtgcagtggcgctatcacagctcactgcagcctggatctcccaggcttaaatgatcctcccacctcagcatcctgagtagctgggactacaggcgcccactaccatgcccggcagattttttgtacttttatttttagtaaagtctattttagtttcactatgttgcccaggctggtcttgaactccagagctcaagcaatcctgtctgcattagcccaccaaactgctaggattacaagggtgagccacggtgcctggctaatatggtagctattgatagcttactatgtatcagatcctatttatttatttatttttgagacagagtctcaccctgtcacctgtgctggagtgcagtggcatgatcttggctcactgccacctccgcctccttggctcaagctgagtagctaggactacagtggtgagccaccatgcccagctaatttttttttttttttttttttttgatagagatgggatttcatcatgttgtccaggctggtcttgaactcctgacctcaagtgatctgcccacctcggcctcccaaagtgctgggattacaggtgtgagcaactgcacctggcccatcaggtgctgttttaaaggctttatatgaatttaataacatatgtcaataggatcgattctatcattatttgcctttttttttttttttttttttgaggcagagtctccccgtcacccaggatggactgcagtggcgcaatctcggctcactgcaacctccacctcccgggtccaagtgattctcctgcctcagcctcccaagtagctgggactacaggcgcccgccaccatgcctggctaatttttgtatttttagtagagatggggtttcatattggccaggctggtctcgaacttctgactttgtgatccgcccgcctcggcctcccaaagtgctgggattacaggcatgagccaccgtgcccggcccattatttcccttttacactcaagaaaattgaggcccagtgaggttaagtgacttgcccaaggtcacacagcgtggaaccaggcagtctggcttcagggtccacacttaacctttgagctatccctggctcctacccaaattcccaaactcacctggcctagctctctgcagggacagtgcttgtaaagaggcatttggctgtgatctccccacctcccagggctggtctggtccccctgccatttgtcctcccttcacccagtcctctagggccctcattgctgactcaccttcgttcacaggggccatgtctgttggggatgctggggggctggggtaggggtttggggttgggtctggggctgtgggggcagctggggctgtggttgtgattgtggctggggctgtggttgtggttggggctgcagcttaggcgggggtgctcgggtgaagaggggggacccagggagcatggcgcggctggccccgtgctcccagaaggcgttctgcagcttgaagatcatgctgagggggatgggacgctggcgcggggccccgcggggctgggggctggaggggggcatggggatgcggctgacgggctgccagctgcgaggcaaagtgcccgacggccccgcggagcccagcgagcgccggtagctgcccgcgtctgaacgccggtcgctggccagaggagagaccttgtaattgcgcggcagggtggcgctagtgaggttgtcctggggaagagggaagggagaaggggatcgggtgagagagggaaggtggaggggaggtaaagacaaaagacgagaagggagaggaggtgagggaagccctgggagtgagggagaagaaagggtgaggaaggagcagaaacccagcacagtgaagggagagcgtgggaacgggcgccgagacccagatcgcagccccgagggggagactggccttgaccccgctcccccaccccactcctcgaccttccccagcctctcctccccaggcgtcgcctcctcaccttgccggtgccccccagtccatccaggctgctctccctccaaggcaacagctgcaggctcggcgaggcaggccttgcgaagacgtccaggcctgcggggcgggaatcattagggtctgtggggctgcctctcctccgggtcctccattccccgggcctccaccactcacgttcatagctcgctgtctgcgaaggcttcttctcgtacgccacgtccaggtcagactcgttccaggctttcggaggccgccggcgcagcgtcaggtcgtctggggagaagtttccagggaggatgagacgggaggggtggcgagccccggatcctgcccgctttgaccccgcgagtcaaaggccccgcgaggggcccctgggttcaccttgcgcgcgcagaggcggggcgaatgcgctgccgccggagcctagcagggagctcccgaaggcggacgctggcgcgtcgtaggctgtggcaggggggcgcggtgacggcccacgctcggggaagaaggcctggggcccctccgccagggggctgccgcggggggagcctgcgcggcccaggaagtcgaaaggcgtggggggaccctgctggcggagcgggcctggcccgggccgcggggagggcgcacggccgagggagctgcctgcgccatcgaaggcgcggggccggggcgaggtcgcgcggtccaggctgccgtaggcgtccggctgcaggtagagcggggtgcgcggcgacgacggccgtcccttgggggacagcgggctgtaggggtgtagggttggggcactctctgatcgtccgaacggggtgtctgcgccgtcggtggccgccttccggggggaccctcggctgccgaagggctcagggatcgagctggagctgtaccggggcggctgtggggaggccagggcattgagggatggatcaaaggagacattagtggaagggttggtgtgtgggcgggggtgtcaagagagatcactggaggtcaacccagaggaggctgaccggccatggaaattcaggcacagagagcccaggtgagtagtggtggggagacagccctgaatcagcactgtggctagcccattactctatgtcacctttatgccacttaggtaaacacctctttccttctgagggtccctttagatgtccacttccactggtcccctcttttctatttctttctttctttctttctctctctttcttttctttctttctttcctctctctccttccttcctttctctctctctccttccctccctccctccctccctgcttgcttgctttctctctctctctttctttctttctttctttctttctttctttctttctttcttttctatctcggctcattgcagcctcaacctccctggcttagtgtgatcctcccacttcagcctcccaagtagctgggattacaggtatgcaccaccacacctggctaacttttgtatttttagtagagacagggtttcaccatgttagccaggctggtcttaaactcctgacctcaagtgatccgcctgtctctgaaagtgttgagattacaggcgtgaaccaccgtgcccagccagatttttaaaaaatcatttgtagaggctggtctcaaactcttagtctcaagcaattctctcacctcgccttccaaagtgctgggattccaggtctgagccatcgcgcctggcctggtccccttttttcaagttcccttgaagagcccacaacctgcataactatatggggcaattttgcctgaaatccaggcctctggtctggactgtggcgagaggctggctttggagatcaaggtgggaaccaggcttaccctagaagggggtccggcctgcgggccaggaggcgcgggagagtctgaccacagcgactccagctgcttggtcagttcatccaccttggccgccgccgtgtccagctccatctgcttcagatccatgtgtttcatggccagcgctgggaaggtgggagtggaggtaaggacctggcctcctggcaggggccggcctcagcacccctcgcccgctgccgaggtccccgcctcgccagccccgccccctactccagcttacactggaagttcatgtccagaaagtcccgcgcgctctggaatgcctcgctgtccatggtgccggccggagcgggcgcctgcatggtggggagggagggagctggctaagaccccgcccctctagaccccgccctcagggagtcagacgccgtcaggagcgggacaacgcctcaactcagttccttcccctggaagccctttaccctttcacctccccagctgggaaatgccaactcctccaaagccaagtccatgcgccacggagaagtccaaacccagtctaaaacctccggaattcactttctctttctttttttcttttctttttttttttttttttgtgtatgtgtgtgagacagagtctcgctctgtcgcccaggcgggagtgcaatgacgcgatcttggctcactgcaacctccgcctcccgggttcaagcaaatcttctgcctagctgggactacaagcgcgcgccattatgcccggctaatttttgtagttctgggattacaggagtgagtctccgcgcccggccgtgtccatctctttatctcagtcctaagacctgaatcactccttgaacaattatctattgatcacctacaatgtgccggtaaacataggatggaataactatgaattactgaatgtttactagggaccaggacgcactgtgctagatcctgtttttgtttgtttttgagatggtgtctcgcattttcgcccaggctggagtgcagtggcgcgatctcggctcactgcaagctccgcctccagggttcatgccagtctcctgtctcagcctcccgagtagctgggactacaggcgcctgccaccatgcctggctaaatttttgtatttttagtagagacggggtttcaccgtgtcagccaggatggtctcgatctcctgaccgcgtgatccatctgcctcggcctcccaaagtgctgggattacaggcgtgagccaccgcgcccggcccttgtttttgttttttaataataattctgctgtctgctgtgtactagaacccatgcctactgcttggggtataatgtagtaaatgtagtaaaaacaatatccgccgggcgcggtggctcacgcctgtaattccagcactttgggaggccaaggagggcggatcacgaggtcaggagagcgagaccatcctggctaacatggtgaaaccccgtctctactaaaaataccaaaaattagccaggcgtggtgatggacgcctgtagtcccagctactcgggaggctgaggcaggagaacggcgtgaacccgggaggtggagcttgaactgagcggagatcgcgccactgcactccagcctgggcgacagtgcgagactccgtcttaaaacaaacaaataaataaatatgtttaaaacaacaacaacaataaccagccaggcgcggtggttcactcctgtaacccgagcactttgggaggccgaggtggatggatcgcttgaagccaggagaccagcctggccaatatggtgaaaccccgtctctacaaaaaaatacaaaagttagctgggcatggtggcatgtgcctgtaatcccagctactcaggaggctgaggcacaaggctcacttgaacctgggaggcacaggttgcagtgagcatagattgtgtcactgcactgcagcttgggtgacagagcgaggctctatttaaaaaaaaaaaaattaattgaggggccactcccttctagagtggtgagaaatgccgtgcaccgaaagcttcatttgatggtcaaaaccaccctagcaggcaagaaagcatggctcagaaacatatgttcaaggtcaccctgcaagaagtcggtagtaatcggtttcacacccgcatctaacttattctgggtcatctctaccagattagaggggtcctagagggaagcgactgctcagcttcctttccctagggtccccattcagtggaggtctggctctcactgacccattgttagcaagaggaacagggaggtggccaggggtggaggggcagctgtggtcactggcccagtgggagggagctaggccactaggaaccggtcaggccagcaccatccctatccccatgctagccaccacacccaccagctctgccacctccctgctgcatcgaccacttagctctggcagtataggcagcagggcaggctggggcatgctgatacccgcctctgtctgggaagtcgaaggaacagaacctgttcaggctggcggctcatttggatgaacagggagtgtgtgaccttgggcgttgagtcctctccactccctgggcctcagtctccccaacatcaaagaagaaggcaaatcacctttttttttttttttgagatagggtctcgctctgtaacccaggctacaattgtgactcactacagcctcttgacctcccagctcaagtggtcctcccacctcagcctcctgagtagctgagactataggtatagcctcgcaccaccacacccagctaattttttttttttttttttttttttttttttttttgagacggagtcttgctctgtcgcccaggctggagttcagtggcgggatctcggctcactgcaagctccgcctcccgggttcacgccattctcccgcctcagcctcccaagtagctgggactacaggcgcccgccactacgcccggctaatttttgtattttagtagagacggggtttcaccattttagccgggatggtctcgatctcctgacctcatgatccgcccgcctcggcctcccaaagtgctgggattacaggcgtgagccaccgcgcccggccacccagctaattttttaaaaacattttgtacactttgggaggctaaggcgggaggatcacgaggtcaggagctcgagaccatcctggctaacacaggtgaaaccctgtctctactaaaaaatacaaaaaaattagctgggcgtggtggcgggcgcctgtagtcccagctactcgggaggctgaggcaggagaatggtgtgaaccagggaggcggagctttcagtgagccgagatcgcgccactgcactccagcctcggagacagagcgagactccgtcccaaaaaaaaaaaaaaaaaaaatttgtagagacagatcaagtctcactttgttgctcaggctggttttgaactcctgggctcaagcaatcctcccgcctcagcctcccaaagtgctgagattacaggcatgagccaccacacctggccaaatcagctattctgaaaggcccctttaatctctatgagccccagactttcaaactgtaaggaccttaggactgtaactaaagttctacagagcctaaacccctcagctaaagagcctattgttggaaagttctgagtccaagattctatctttggaacattctagaattctccaatttgtctaacccagaattctgagtctttctgtaccacattctacctaacccagggttgcactgctctggaagtctagatggatggtatagtgcagctggtaaaagcatgagtaagaagtcagacttcaaaaattcaaatctgagggccgggcatggtagcttctgcctgtaatccttgcactttgggaggccgaggggggaggatcacttgaggccaggagttcaagaccaacatggccaacacaatgagaccccatttcttaaaaaaaattaaaataaaatcatcaaatctggcagcaccaccgtccaaccctgaccacagtacctcagtctcgtaatccgtaaaatggggatgaaagttcacctcataggactactgtaagaatccacctggtcagaaggtgcaggaagaattcagagctctgagaattgaggcctcaggaagaagagactacaggaataaaaactcgggcatttagaatttcagagatacacaaacaatactttgttaactgttaaaatagataaatgagcaagtctgtgcagccctaatgccagctgtaagtgactctttttttttcttttggtagagatttagtctctctcgcgcctgtggttaggctggtctcgaactcctagcctcatgggatcctccccggctcgatctcccaaagtattgggattacaggcgtgagcacggcgccatgatccccaaatttccaagattctcagattccatactgacattctctggctctcaggaaatgccaaccctgggtgtggggctgtcgcggggacaggcggtggggacgtcggagccaccagggggcggtcacgcccggacccccgccaggagggcggactgcgcctgagctcaggcccggggaatgcgcagcgggcccgggcaggtgctgtacatcccggggcaagggagctgggccgggcggggtacaagggcggggcgcgggggtggcgcgggccgtgtgtctgttcccaggcctctgcccctgacctctgcctccgagtcctctcccatgtgctcccctctagctctagctccgagctctcccgcgggctctgggccagccgcaggtactctcccctgggctcctctctccgctccacccctggctctccttccctggcctcctctgcaccccagccaggttctttagggctaaggatcctgtggacttcctggaggagtcatcttcagtaggaaccgggtcagagagccagactgagctgggaacacccaggctggactcctacagccctgtcgggtcacactgaatctggagaggctccactgtctctgggactcggtttcctcctttgtggacgtctatggaatgggctagggcctttcttgctctaagcctctacttgggcttgttatttagcttctctgtgcctgtttcctcatgtggaccatgggaagaattaataccttcgcctcaaaggggtatgaggattgagtgacataatttataagccgtgattagaacaatgcagtgcgcgaaataaagttcacacatacaggattcataattaccagatgtccttggctgttcattataataacacagggtctggcaacagagtgaggggtccagactcaatgtaatttttttttcccctaaaagggccctttcaactctttctgagatcatacaagccctgagttttgacacccagggtctcaacttcctgagcccttgcctctcagagtcctaaatttcccctgtacattcctgagtctggccagtgatcaccctcagtcacttagggacgggagggctgggagagccctggaagattccagacagaagctggcaaaagcccagggtgtgggcaatatccactctccagcctccgtttctccactcgtaatgaggagtccttccctggggtcagcaaaccttattcaaagggagacctctcagtcacccaagattcctctagacaatgcgagctttcctacctacctacctaccagctctgagcttggtacacccagagccctgttttggcaaccacggttattatttttaatttcatttcaggttatcatcaaatgcccttcaagcccagacattgggaaacactcctctctcatcagatgctcgcctcccccattctgtttttaatcccccttcttaggacgcatgggggttgagagaacggggagatagacagagggaggtgcctggtcctgccctccccccgcctcaaggacagacagacacctccagaattagcctctgtccctccttatctcccacaataccccaggtcagacagatgggcgtggaggtgacatttctcacctcagggtcagggcaaggagccctgaggcagaaggttagtcagaaaatctggcgggggcggatggaatcccgtcccccagagagctgcagaagaaggaggaggcagaatcctgaccctacaaactctactgcctgtgtgagctccaagcctcagtttaccccttcctctccgtgtaatggttaaatgcccggctatgcaaacctcccagaatccaatagccgctttccggaattctgccctgggttctagaactacctctgcaaacccagctgtttcccaccccataaggcaataggggagcccacctccgccagggggtgccctagggcggatgtcccttctctggttaggcaggtctgacgcccaggttaatgacatgttgggttcgctcagcggcacagaggaggttggagatctgcctcggtgttttctctcctaccccgcccccatccccgagccgaaaagtcgggggagagccgggacacagcctccggagggaccccgggtacctgtcctgctccacttcaggaacccaggctccactatccctgccccacccttaattctgctcagagacctagaagatcggtcgagacagcagcttgaggctggcagggtggtcacccattccaccttgagccccaccagtctgagcctctcatttctgaccaagactcggggattcgaacccctatactacccaaagactcggcttcctagagccccccagttcgagggactcaggaattccagctccaacgtctccccgggatgaaggggtagaatccctccattccaagaattcaggcatccgaacccgctttccttccctccagtaaaacaggcaacggagtttccttctaaggatccaggtgtcggcgcgccccaaattccgccctgggacctggcgtccgagtcccctcccaatcctcccagggacgcgggtgttgggctttttcagggcctctggtccccaggagggtgaaactcacggatccgggcagatcctggcacctgggggcttcctccagctcgggctccggcttggggagcggagaacggggcggggcaggagctgggaacaggttagacgacgtgacttgggctggagggaggcgggtcccggtggggagggggagccaaggtcgcctcgagcaccttgggacttgtagtcccggagggacaggacgtagcccaagacgatcccatttggattcacccagagtccatttcacagacaggaagggcgaggcccagaagccgagagcgaccaggccagggagatacagaagagccgagacgcctgcctcgctgtggctggagactgactcctgagcccttgccccaccccttcaggcgcactatcccctttcctgatcagtatcccccagggtctctgagcccgaatctccccgtcgataaaaagcgcgggttggatcttcaaaggatgtcccagcaagagttcaaaatcttagtttggactacaacccccagcagcctccgcgaccgccctcgggcgactctttgcctcgggtcctgtgggaattgtagtcctggagcccgcagggctgcaccccggtgtctctctcgcccacgcgaaggaaaccgtctggagatcctggataggggaaacatttccccttccccttgaccctccctccgctctggaaagcctctcccacctggggagaaggggtgccccaattctggagtaggatcctaaatcttggcagagggggcgggaagtggcgctgacacactggccaggaatgcagtcgggtcaccctgtctagccaccgtctcgcggctccaaccgccgcccaacgcggggcggccccagtgggaagggaagtgggtgcgtcccccaaatctgtgtccacgtgccgctgtttacacgctccctggggcagggaggagtcgccgatcaggtcccttcctgaaagtcatcgaggtttcccacgcatgagactaaacccccgagggcatctacaagtcccatttgatccacaaacgctacaccgtgcccagcaccactccacgcgtgtggggctcctgggtccgaggctccgccctcgagaaccacaagctcctccccctatgtttcccgctcccccggagtccagaagccccgcccctggctggaacttcacgccctccggacggattgcccctatttctccattttcccgcttctcccagtcaagttctgaacttgtgaggcatctgggcctccccagaagacatttaacacagaaagcacagccctactaactagtattcttacctgtctcttcaagaatttcagaccaatcgaccgtcctgtctctttaaggcttaggaagagcagtgtggctgcccctttaaggaggcgttgcaacaaaccatattggacagacgatgggggcgacccatcgggacccgacgggcctctgactccagcaatacagcgaatcagcggctttcgggaatacatttttcggaaaaagacttcttcctcggttttctgctctgcacacgttgaaattttccccagtttttcctgcagatcgggagtcgagcaatgcctacccccgcgctcccgcaccagttgggcgctcccggatgatgccctacccctttggatccacgtggtctgcaacctggtgcgagcagcccgggctacagggttgcctgaggtgtgggtcccaggatggaggagccccaggccggcggtgagggtgcgggttgacggggtgcggagggtgcgttggtggaaggagaaaggggcgtccgagagggttcgggcggaaaaggaggcgtacctgcaagcaggacttgcgaagagcgtgcattcccagtgggcgaacgggaattcgaacggagagagggttatcttgtggggggctacccgtggagagcaaggcgcccccaggggttggatcggtgaaattgaggtcgcccctggggaacaggtgggcagaaaggagaaaccaggttgaggggactggagtgctcacgaggttaagaccaatggaccgataggcgcgccctgcaagattggaccggcaaggaggtgtcagtcgaccccatttccccttctgctgcagatgctgctcggttctcttgtccccccaactttaccgcgaagcccccagcctcagagtcccctcgtttctccttggaggcgctgacgggtccagatacggagctgtggcttattcaggcccctgcagactttgccccagaatggtgagtggtcttgttgacggaaaagagggtcccggtccagaccccaagagcgggttcttgaatttgtcacaggaaagaattagaggtgagtcacagagcacagtgaaagaaacaagtttattggaaactactcctttacagagtagagtgtcctcagaaagcagggggagaaacccacagccctttgttagtatttctacttataagaaactataaggaactatagttaaacttggagtgtgcagataagctcactaaaggtaggggctattggtgttatccacgaccattaatcctgcaacctaagcttgctcatttatgttatatttaagtaatgggggctgcattcttaggacatttggacattctgcaggcttggtggaacatgttctgtatggccataaatattctgtaattataattggtggtcagcctgggatgtggttattttcaggccataagcatgaaccttgtaagtgcctagctactcactttaagatggagtcactctagtcatgttttattaaaaaccagaggccagccaggcgcagtggctggtgcctgtaatcccatcctttgggaggccgaggcgagcagatcacttgaggtcaggagttcaagaccagcctggccaacatagtgaaattgtctctactaaaaatacaaaaattggctgggcgtggtggcaggtgcctgtaatcccagctacttgagaggctgaggcaggagaatcgcttgaacccaggaggtggacattgcagtgagccgagatcatgccactgcactccagcctaggcaacagagcaagactctctcaaaaaaaaacaaaaaaaaaatcaaaaaaccttccctctcctgttccacttaagcctctgccctccctgtttctctctgtagcttcaatgggcggcatgtgcctctctctggctcccagatcgtcaagggcaaattggcaggcaagcggcaccgctatcgagtcctcagcagctgtccccaagctggagaagcgaccctgctggccccctcaacggaggcaggaggtggactcacctgtgcctcagccccccagggcaccctaaggatccttgagggtccccagcaatccctgtcagggagccctctgcagcccatcccagcaagtcccccaccacagatccctcctggcctgaggcctcggttctgtgcctttgggggcaacccaccagtcacagggcctaggtcagccttggcccccaacctgctcacctcagggaagaagaaaaaggagatgcaggtgacagaggccccagtcactcaggaggcagtgaatgggcacggggccctggaggtggacatggctttggggtcgccagaaatggatgtgcggaagaagaagaagaaaaaaaatcagcagctgaaagaaccagaggcagcagggcctgtggggacagagcccacagtggagacactggagcctctgggagtgctgttcccgtccaccaccaagaagaggaagaagcccaaagggaaagaaaccttcgagccagaagacaagacagtgaagcaggaacagattaacactgagcctctagaagacacagtcctgtccccgaccaaaaagagaaagaggcaaaaggggacggaagggatggagccagaggagggggtgacagttgagtctcagccacaggtgaaggtggagccactggaggaagccatccctctgccccctacgaagaagaggaaaaaagaaaagggacagatggcaatgatggagccagggacggaggcgatggagccagtggagccggagatgaagcctctggagtccccaggggggaccatggcgcctcaacagccagaaggagcgaagcctcaggcccaggcagctctggcagctcccaaaaagaagacgaagaaagaaaaacagcaagatgccacagtggagccagagacagaggtggtggggcctgagctgccggatgaccttgagcctcaggcagctcccacatccaccaagaagaagaagaagaagaaagagagaggtcacacagtgactgagccaattcagccactagagcctgaactgccaggggagggacagcctgaagccagggcaactccgggatccaccaagaagaggaagaagcagagtcaggaaagccggatgccagagacagtgccccaagaggagatgccagggccgccactgaattcagagtctggggaggaggctcccacaggccgggacaagaagcggaagcagcagcagcagcagcctgtgtagtctgcccccgggaaactgaggaactaaagaaagctgaaggtgcccacctgggccaccagaaggtgacacccccagaatccctccccagagactgcaccagcgcagcc

SEQ ID NO: 3 - DNA, 41 bp, Artificial sequence
  Probe
gctctgaaacttactagcccrgtatttatggagaggcattt

SEQ ID NO: 4 - DNA, 46 bp, Artificial sequence
  Single nucleotide polymorphism
gtggtcaaattctcattcatcgtggyccaggcaagcacacttcctc

SEQ ID NO: 5 - DNA, 51 bp, Artificial sequence
  Single nucleotide polymorphism
accctgaggtgagcacctgttccttytccttgcccttagcccagaggtaga

SEQ ID NO: 6 - DNA, 51 bp, Artificial sequence
  Single nucleotide polymorphism
gggcaggggtttgtgcctccaatgarcacaagctccccctgccccccaact

SEQ ID NO: 7 - DNA, 19 bp, Artificial sequence
  Probe
tggctaacacggtgaaacc

SEQ ID NO: 8 - DNA, 23 bp, Artificial sequence
  Probe
ggaatccaaagattctatgatgg

SEQ ID NO: 9 - DNA, 21 bp, Artificial sequence
  Probe
gggaggcggagcttgcagtga

SEQ ID NO: 10 - DNA, 20 bp, Artificial sequence
  Probe
ctgagatcgcaccactgcac

SEQ ID NO: 11 - DNA, 20 bp, Artificial sequence
  Probe
ggttttctgctctgcacacg

SEQ ID NO: 12 - DNA, 20 bp, Artificial sequence
  Probe
cctttctccttccaccaacg

SEQ ID NO: 13 - DNA, 21 bp, Artificial sequence
  Probe
cgggctacagggttacctgag

SEQ ID NO: 14 - DNA, 22 bp, Artificial sequence
  Probe
tctgcaacctggtgcgagcagc

SEQ ID NO: 15 - DNA, 21 bp, Artificial sequence
  Probe
cctaccaccatcatcacatcc

SEQ ID NO: 16 - DNA, 21 bp, Artificial sequence
  Probe
gccttgccaaaaatcataacc

SEQ ID NO: 17 - DNA, 30 bp, Artificial sequence
  Probe
cctctccccaattaagtgccttcacacagc

SEQ ID NO: 18 - DNA, 19 bp, Artificial sequence
  Probe
agccagggaggttgaggct

SEQ ID NO: 19 - DNA, 20 bp, Artificial sequence
  Probe
agacagccctgaatcagcac

SEQ ID NO: 20 - DNA, 19 bp, Artificial sequence
  Probe
gcaatgagccgagatagaa

SEQ ID NO: 21 - DNA, 19 bp, Artificial sequence
  Probe
tggctagcccattactcta

SEQ ID NO: 22 - DNA, 20 bp, Artificial sequence
  Probe
agccccaagaccctttcact

SEQ ID NO: 23 - DNA, 22 bp, Artificial sequence
  Probe
gtcccatagataggagtgaaag

SEQ ID NO: 24 - DNA, 20 bp, Artificial sequence
  Probe
ccctaggacacaggagcaca

SEQ ID NO: 25 - DNA, 20 bp, Artificial sequence
  Probe
ttgtgctttctctgtgtcca

SEQ ID NO: 26 - DNA, 20 bp, Artificial sequence
  Probe
tatcagaaaaggctggagga

SEQ ID NO: 27 - DNA, 19 bp, Artificial sequence
  Probe
gagtggctggggagtagga

SEQ ID NO: 28 - DNA, 19 bp, Artificial sequence
  Probe
gccaagcagaagagacaaa

SEQ ID NO: 29 - DNA, 20 bp, Artificial sequence
  Probe
cctcagatgtcctctgctca

SEQ ID NO: 30 - DNA, 20 bp, Artificial sequence
  Probe
gccacagccccagcaagtag

SEQ ID NO: 31 - DNA, 20 bp, Artificial sequence
  Probe
aggaccacaggacacgcaga

SEQ ID NO: 32 - DNA, 20 bp, Artificial sequence
  Probe
catagaacagtccagaacac

SEQ ID NO: 33 - DNA, 25 bp, Artificial sequence
  Probe
ttagcttggcacggctgtccaagga

SEQ ID NO: 34 - DNA, 26 bp, Artificial sequence
  Probe
acagaattcgccccggcctggtacac

SEQ ID NO: 35 - DNA, 23 bp, Artificial sequence
  Probe
ttgaaactggaactctgagaagg

SEQ ID NO: 36 - DNA, 19 bp, Artificial sequence
  Probe
tggtggatggtgtgaagca

SEQ ID NO: 37 - DNA, 30 bp, Artificial sequence
  Probe
cctttctccaacttcttctccatttccacc

SEQ ID NO: 38 - DNA, 23 bp, Artificial sequence
  Probe
ggggatcatgtcgtcaatggact

SEQ ID NO: 39 - DNA, 20 bp, Artificial sequence
  Probe
atgccctgtaggttcaatgg

SEQ ID NO: 40 - DNA, 20 bp, Artificial sequence
  Probe
tggaggtctttaggggcttg

SEQ ID NO: 41 - DNA, 24 bp, Artificial sequence
  Probe
ggctggtccccgtcttctccttcc

SEQ ID NO: 42 - DNA, 22 bp, Artificial sequence
  Probe
tctctgttgccacttcagcctc

SEQ ID NO: 43 - DNA, 22 bp, Artificial sequence
  Probe
gtcctgccctcagcaaagagaa

SEQ ID NO: 44 - DNA, 22 bp, Artificial sequence
  Probe
ttctcctgcgattaaaggctgt

SEQ ID NO: 45 - DNA, 22 bp, Artificial sequence
  Probe
atcctgtccctactggccattc

SEQ ID NO: 46 - DNA, 22 bp, Artificial sequence
  Probe
tgtggacgtgacagtgagaaat

SEQ ID NO: 47 - DNA, 23 bp, Artificial sequence
  Probe
tggagtgctatggcacgatctct

SEQ ID NO: 48 - DNA, 23 bp, Artificial sequence
  Probe
ccatgggcatcaaattcctggga

SEQ ID NO: 49 - DNA, 22 bp, Artificial sequence
  Probe
cacacctggctcatttttgtat

SEQ ID NO: 50 - DNA, 21 bp, Artificial sequence
  Probe
tcatccaggttgtagatgcca

SEQ ID NO: 51 - DNA, 21 bp, Artificial sequence
  Probe
aggctcaacaaggaaaaatgc

SEQ ID NO: 52 - DNA, 22 bp, Artificial sequence
  Probe
gctagacagtcaaggagggacg

SEQ ID NO: 53 - DNA, 25 bp, Artificial sequence
  Probe
aaagggtgggtgtgggagacattgg

SEQ ID NO: 54 - DNA, 22 bp, Artificial sequence
  Probe
aaaccaacctaggcaccccaaa

SEQ ID NO: 55 - DNA, 18 bp, Artificial sequence
  Probe
cagtgtccaaagagcacc

SEQ ID NO: 56 - DNA, 17 bp, Artificial sequence
  Probe
ctacccctttagcgacc

SEQ ID NO: 57 - DNA, 21 bp, Artificial sequence
  Probe
tcctgcccccagagcgtcacc

SEQ ID NO: 58 - DNA, 25 bp, Artificial sequence
  Probe
gtacggtccacataattttggagga

SEQ ID NO: 59 - DNA, 22 bp, Artificial sequence
  Probe
cgacgaacttctctgaagcgaa

SEQ ID NO: 60 - DNA, 18 bp, Artificial sequence
  Probe
agcgacacgggcatctgg

SEQ ID NO: 61 - DNA, 22 bp, Artificial sequence
  Probe
atgagcgtccacctcctgaacc

SEQ ID NO: 62 - DNA, 22 bp, Artificial sequence
  Probe
aggcagcagcatcgtcatcccc

SEQ ID NO: 63 - DNA, 18 bp, Artificial sequence
  Probe
tgcatagctaggtcctgc

SEQ ID NO: 64 - DNA, 35 bp, Artificial sequence
  Probe
aactgacraaactagctctatggggtggtgccgca

SEQ ID NO: 65 - DNA, 23 bp, Artificial sequence
  Probe
ctggctctgaaacttactagccc

SEQ ID NO: 66 - DNA, 19 bp, Artificial sequence
  Probe
gctggactgtcaccgcatg

SEQ ID NO: 67 - DNA, 17 bp, Artificial sequence
  Probe
ggagcagggttggcgtg

SEQ ID NO: 68 - DNA, 22 bp, Artificial sequence
  Probe
tgccctcccagaggtaaggcct

SEQ ID NO: 69 - DNA, 21 bp, Artificial sequence
  Probe
ccctcccggaggtaaggcctc

SEQ ID NO: 70 - DNA, 20 bp, Artificial sequence
  Probe
gatcaaagagacagacgagc

SEQ ID NO: 71 - DNA, 16 bp, Artificial sequence
  Probe
gaagcccaggaaatgc

SEQ ID NO: 72 - DNA, 20 bp, Artificial sequence
  Probe
ggacgcccacctggccaacc

SEQ ID NO: 73 - DNA, 19 bp, Artificial sequence
  Probe
cgtgctgcccaacgaagtg

SEQ ID NO: 74 - DNA, 15 bp, Artificial sequence
  Probe
gccccgtcccaggta

SEQ ID NO: 75 - DNA, 46 bp, Artificial sequence
  Probe
cctggcggtggccgtcaccagctttygggggtgtttgggaagctgg

SEQ ID NO: 76 - DNA, 41 bp, Artificial sequence
  Probe
ctccagccccactgttccctrggccctattggtccccctgg

SEQ ID NO: 77 - DNA, 46 bp, Artificial sequence
  Probe
acaaggaggaggcagaagtgaggttsaaacccactgcccaatctta

SEQ ID NO: 78 - DNA, 46 bp, Artificial sequence
  Probe
ccaacacggtgaaaccccgtctgtaytaaaaatacaaaaattagcc

SEQ ID NO: 79 - DNA, 46 bp, Artificial sequence
  Probe
aatccaggaccccataatcttccgtyatctaaaacaataatggtga

SEQ ID NO: 80 - DNA, 46 bp, Artificial sequence
  Probe
cccaagggggcgaggggagggtgaargggtgggacgggggcagccg

SEQ ID NO: 81 - DNA, 46 bp, Artificial sequence
  Probe
gaagtgagaagggggctgggggtcggcgctcgctagcgggcgcggg

SEQ ID NO: 82 - DNA, 46 bp, Artificial sequence
  Probe
cgcacgcgcagtatcccgattggctstgccctagcggattgacggg

SEQ ID NO: 83 - DNA, 49 bp, Artificial sequence
  Probe
aactcctgggttcgatcaatactcagacaatcttggcaggcgcaggagg

SEQ ID NO: 84 - DNA, 46 bp, Artificial sequence
  Probe
gctgggattacaggcttgagccaccrcgcccggcctgcaaagccat

SEQ ID NO: 85 - DNA, 45 bp, Artificial sequence
  Probe
ttttgtatctttagtagagacaggktttctccatgttggtcaggc

SEQ ID NO: 86 - DNA, 48 bp, Artificial sequence
  Probe
gcctcagcctcccgagtagctgagactmcaggtgcccgccaccacgcc

SEQ ID NO: 87 - DNA, 48 bp, Artificial sequence
  Probe
tgaaattgtaggttgagaggccaggcgyggtgctcacgcctgtaattt

SEQ ID NO: 88 - DNA, 41 bp, Artificial sequence
  Probe
gtttataaacattaaaccagwgctgtgtgaaggcacttaat

SEQ ID NO: 89 - DNA, 44 bp, Artificial sequence
  Probe
ccgtctctattaaaaatataaaamaatttagccgggtgtagcgg

SEQ ID NO: 90 - DNA, 39 bp, Artificial sequence
  Probe
gggaggctcgaggcgggcrgattgcatgagctcaggatt

SEQ ID NO: 91 - DNA, 41 bp, Artificial sequence
  Probe
tcccaagtttcagggcccaakattctcaaatcacaggattc

SEQ ID NO: 92 - DNA, 40 bp, Artificial sequence
  Probe
tgcagtgagctgagatcgcrccactgcactccagcctggg

SEQ ID NO: 93 - DNA, 40 bp, Artificial sequence
  Probe
tcttaggacgcatgggggtkgagagaacggggagatagac

SEQ ID NO: 94 - DNA, 39 bp, Artificial sequence
  Probe
ctgggttctagaactaccyatgcaaacccagctgtttcc

SEQ ID NO: 95 - DNA, 48 bp, Artificial sequence
  Probe
attctgccctgggttctagaactacctmtgcaaacccagctgtttccc

SEQ ID NO: 96 - DNA, 44 bp, Artificial sequence
  Probe
gctgtttcccaccccataaggcartaggggagcccacctccgcc

SEQ ID NO: 97 - DNA, 42 bp, Artificial sequence
  Probe
gacctagaagatcggtcgagayagcagcttgaggctggcagg

SEQ ID NO: 98 - DNA, 46 bp, Artificial sequence
  Probe
ctggccaggaatgcagtcgggtcacyctgtctagccaccgtctcgc

SEQ ID NO: 99 - DNA, 41 bp, Artificial sequence
  Probe
gggaggagtcgccgatcaggycccttcctgaaagtcatcga

SEQ ID NO: 100 - DNA, 41 bp, Artificial sequence
  Probe
gcagcccgggctacagggttrcctgaggtgtgggtcccagg

SEQ ID NO: 101 - DNA, 41 bp, Artificial sequence
  Probe
tagaaatactaacaaagggcygtgggtttctccccctgctt

SEQ ID NO: 102 - DNA, 43 bp, Artificial sequence
  Probe
acaggagagggaaggttttttgwttttttttttgttttttttt

SEQ ID NO: 103 - DNA, 44 bp, Artificial sequence
  Probe
gaagaggaagaagcccaaagggamagaaaccttcgagccagaag

SEQ ID NO: 104 - DNA, 44 bp, Artificial sequence
  Probe
gcgcctcaacagccagaaggagcgragcctcaggcccaggcagc

SEQ ID NO: 105 - DNA, 40 bp, Artificial sequence
  Probe
ttgagactctctgtttgatrcttcactcagaaggtgcttc

SEQ ID NO: 106 - DNA, 42 bp, Artificial sequence
  Probe
aggccaggctcctgctggctgsgctggtgcagtctctgggga

SEQ ID NO: 107 - DNA, 40 bp, Artificial sequence
  Probe
cccctataccctcaagcatytatccattgagttacaaaca

SEQ ID NO: 108 - DNA, 41 bp, Artificial sequence
  Probe
accatcccccgccttccgttmgtccggcccccgaggctagc

SEQ ID NO: 109 - DNA, 20 bp, Artificial sequence
  Primer
ggttttctgctctgcacacg

SEQ ID NO: 110 - DNA, 20 bp, Artificial sequence
  Primer
cctttctccttccaccaacg

SEQ ID NO: 111 - DNA, 22 bp, Artificial sequence
  Probe
tctgcaacctggtgcgagcagc

SEQ ID NO: 112 - DNA, 21 bp, Artificial sequence
  Probe
cgggctacagggttacctgag

SEQ ID NO: 113 - DNA, 23 bp, Artificial sequence
  Primer
ttgaaactggaactctgagaagg

SEQ ID NO: 114 - DNA, 19 bp, Artificial sequence
  Primer
tggtggatggtgtgaagca

SEQ ID NO: 115 - DNA, 30 bp, Artificial sequence; identical to SEQ ID NO: 37
  Probe

SEQ ID NO: 116 - DNA, 23 bp, Artificial sequence
  Probe
ggggatcatgtcgtcaatggact

SEQ ID NO: 117 - DNA, 20 bp, Artificial sequence
  Primer
aggaccacaggacacgcaga

SEQ ID NO: 118 - DNA, 20 bp, Artificial sequence
  Primer
catagaacagtccagaacac

SEQ ID NO: 119 - DNA, 28 bp, Artificial sequence
  Probe
tggcgacgtaattcccgactatgtgctg

SEQ ID NO: 120 - DNA, 19 bp, Artificial sequence
  Probe
cgcaacgtgccctgggaat

SEQ ID NO: 121 - DNA, 21 bp, Artificial sequence
  Primer
aggctcaacaaggaaaaatgc

SEQ ID NO: 122 - DNA, 22 bp, Artificial sequence
  Primer
gctagacagtcaaggagggacg

SEQ ID NO: 123 - DNA, 25 bp, Artificial sequence
  Probe
aaagggtgggtgtgggagacattgg

SEQ ID NO: 124 - DNA, 22 bp, Artificial sequence
  Probe
aaaccaacctaggcaccccaaa

SEQ ID NO: 125 - DNA, 22 bp, Artificial sequence
  Primer
cgacgaacttctctgaagcgaa

SEQ ID NO: 126 - DNA, 18 bp, Artificial sequence
  Primer
agcgacacgggcatctgg

SEQ ID NO: 127 - DNA, 22 bp, Artificial sequence
  Probe
atgagcgtccacctcctgaacc

SEQ ID NO: 128 - DNA, 22 bp, Artificial sequence
  Probe
aggcagcagcatcgtcatcccc

SEQ ID NO: 129 - DNA, 20 bp, Artificial sequence
  Primer
atgccctgtaggttcaatgg

SEQ ID NO: 130 - DNA, 20 bp, Artificial sequence
  Primer
tggaggtctttaggggcttg

SEQ ID NO: 131 - DNA, 24 bp, Artificial sequence
  Probe
ggctggtccccgtcttctccttcc

SEQ ID NO: 132 - DNA, 22 bp, Artificial sequence
  Probe
tctctgttgccacttcagcctc

SEQ ID NO: 133 - DNA, 19 bp, Artificial sequence
  Primer
tggctaacacggtgaaacc

SEQ ID NO: 134 - DNA, 23 bp, Artificial sequence
  Primer
ggaatccaaagattctatgatgg

SEQ ID NO: 135 - DNA, 21 bp, Artificial sequence
  Probe
gggaggcggagcttgcagtga

SEQ ID NO: 136 - DNA, 20 bp, Artificial sequence
  Probe
ctgagatcgcaccactgcac

SEQ ID NO: 137 - DNA, 18 bp, Artificial sequence
  Primer
cagtgtccaaagagcacc

SEQ ID NO: 138 - DNA, 17 bp, Artificial sequence
  Primer
ctacccctttagcgacc

SEQ ID NO: 139 - DNA, 21 bp, Artificial sequence
  Probe
tcctgcccccagagcgtcacc

SEQ ID NO: 140 - DNA, 25 bp, Artificial sequence
  Probe
gtacggtccacataattttggagga

SEQ ID NO: 141 - DNA, 20 bp, Artificial sequence
  Primer
gatcaaagagacagacgagc

SEQ ID NO: 142 - DNA, 16 bp, Artificial sequence
  Primer
gaagcccaggaaatgc

SEQ ID NO: 143 - DNA, 20 bp, Artificial sequence
  Probe
ggacgcccacctggccaacc

SEQ ID NO: 144 - DNA, 19 bp, Artificial sequence
  Probe
cgtgctgcccaacgaagtg

SEQ ID NO: 145 - DNA, 20 bp, Artificial sequence
  Primer
ttgtgctttctctgtgtcca

SEQ ID NO: 146 - DNA, 20 bp, Artificial sequence
  Primer
tatcagaaaaggctggagga

SEQ ID NO: 147 - DNA, 20 bp, Artificial sequence
  Probe
aggaccacaggacacgcaga

SEQ ID NO: 148 - DNA, 20 bp, Artificial sequence
  Probe
catagaacagtccagaacac

SEQ ID NO: 149 - DNA, 22 bp, Artificial sequence
  Primer
cacacctggctcatttttgtat

SEQ ID NO: 150 - DNA, 21 bp, Artificial sequence
  Primer
tcatccaggttgtagatgcca

SEQ ID NO: 151 - DNA, 23 bp, Artificial sequence
  Primer
tggagtgctatggcacgatctct

SEQ ID NO: 152 - DNA, 23 bp, Artificial sequence
  Primer
ccatgggcatcaaattcctggga

SEQ ID NO: 153 - DNA, 22 bp, Artificial sequence
  Primer
gtcctgccctcagcaaagagaa

SEQ ID NO: 154 - DNA, 22 bp, Artificial sequence
  Primer
ttctcctgcgattaaaggctgt

SEQ ID NO: 155 - DNA, 22 bp, Artificial sequence
  Primer
atcctgtccctactggccattc

SEQ ID NO: 156 - DNA, 22 bp, Artificial sequence
  Primer
tgtgaacgtgacagtgagaaat

SEQ ID NO: 157 - DNA, 22 bp, Artificial sequence
  Primer
gtcccatagataggagtgaaag

SEQ ID NO: 158 - DNA, 20 bp, Artificial sequence
  Primer
ccctaggacacaggagcaca

SEQ ID NO: 159 - DNA, 18 bp, Artificial sequence
  Primer
tgcatagctaggtcctgc

SEQ ID NO: 160 - DNA, 19 bp, Artificial sequence
  Primer
gccaagcagaagagacaaa

SEQ ID NO: 161 - DNA, 19 bp, Artificial sequence
  Primer
gagtggctggggagtagga

SEQ ID NO: 162 - DNA, 35 bp, Artificial sequence; identical to SEQ ID NO: 64
  Primer

SEQ ID NO: 163 - DNA, 21 bp, Artificial sequence
  Primer
cctaccaccatcatcacatcc

SEQ ID NO: 164 - DNA, 21 bp, Artificial sequence
  Primer
gccttgccaaaaatcataacc

SEQ ID NO: 165 - DNA, 30 bp, Artificial sequence; identical to SEQ ID NO: 17
  Primer

SEQ ID NO: 166 - DNA, 22 bp, Artificial sequence
  Primer
cgcaaaaacttgtgtattcacc

SEQ ID NO: 167 - DNA, 22 bp, Artificial sequence
  Primer
cccatttttatcatcagcaacc

SEQ ID NO: 168 - DNA, 23 bp, Artificial sequence
  Primer
ctggctctgaaacttactagccc

SEQ ID NO: 169 - DNA, 19 bp, Artificial sequence
  Primer
gctggactgtcaccgcatg

SEQ ID NO: 170 - DNA, 17 bp, Artificial sequence
  Primer
ggagcagggttggcgtg

SEQ ID NO: 171 - DNA, 22 bp, Artificial sequence
  Probe
tgccctcccagaggtaaggcct

SEQ ID NO: 172 - DNA, 21 bp, Artificial sequence
  Probe
ccctcccggaggtaaggcctc